(19) United States
(12) Patent Application Publication
(10) Pub. No.: US 2026/0083856 A1
(43) Pub. Date: Mar. 26, 2026

(54) THIAZOLIDINE LINKERS FOR PROTEIN-DRUG CONJUGATES AND USES THEREOF

(71) Applicant: R.P. Scherer Technologies, LLC, Carson City, NV (US)

(72) Inventors: Xiao Cai, San Ramon, CA (US); Stepan Chuprakov, Walnut Creek, CA (US); Reji Nair, Fremont, CA (US); Ayodele Ogunkoya, Berkeley, CA (US); Matthew Smith, Oakland, CA (US)

(21) Appl. No.: 19/325,974

(22) Filed: Sep. 11, 2025

Related U.S. Application Data
(60) Provisional application No. 63/697,388, filed on Sep. 20, 2024.

(51) Int. Cl. A61K 47/68; C07K 16/32

(52) U.S. Cl. CPC A61K 47/6851; A61K 47/6803; A61K 47/6889; C07K 16/32; C07K 2317/40; C07K 2317/94

(57) ABSTRACT

The present disclosure provides thiazolidine (Tz) linkers for protein-drug conjugates. In addition, the disclosure also encompasses compounds useful for producing such protein-drug conjugates, as well as methods for production of such protein-drug conjugates. The disclosure also encompasses methods of using the protein-drug conjugates for the treatment of a disease or disorder in a subject.

### CROSS-REFERENCE TO RELATED APPLICATIONS

This application claims the benefit of U.S. Provisional Application No. 63/697,388, filed Sep. 20, 2024, the disclosure of which is incorporated herein by reference in its entirety.

### INCORPORATION-BY-REFERENCE OF SEQUENCE LISTING XML

A Sequence Listing is provided herewith as a Sequence Listing XML, “RDWD-058_SEQ_LIST” created on Sep. 10, 2025, and having a size of 23,205 bytes. The contents of the Sequence Listing XML are incorporated by reference herein in their entirety.

### INTRODUCTION

Antibody-drug conjugates (ADCs) are a relatively new but promising class of biotherapeutics that rely on the targeting affinity of antibodies for effective delivery and release of potent biologically active molecules at the targeted site or tissue. ADCs have proven to be especially effective in treating malignancies that overexpress various tumor-associated proteins on the cell surface that are readily recognized by the antibody portion of ADC. Once the ADC has bound to the cell, it is internalized and processed, eventually bringing the active cytotoxic payload to its intracellular target, leading to cell death. This therapeutic approach has an obvious advantage over conventional chemotherapy due to its selective and discriminative nature which permits sparing healthy tissue to improve drug tolerability and quality of life for patients.

Significant advances have been made over the past two decades to further improve both efficacy and tolerability of this class of chemotherapeutic agents. It has been widely recognized that the commonly observed off-target toxicities associated with ADCs often result from conjugate instability, while the lack of efficacy is often attributed to poor pharmacokinetic properties of the ADC that lead to its rapid clearance. The problem of unsatisfactory pharmacokinetic profiles, which likely result from the excessive hydrophobicity of conjugate, are being addressed by the design of special hydrophilic linkers that mask the payload's hydrophobic nature and/or by employing alternative conjugation techniques to avoid the use of hydrophobic conjugation moieties. It has also been demonstrated that both ADC stability and PK profiles can be dramatically improved by employing site-specific conjugation methods, especially those that enable flexible placement of the conjugated moiety within the antibody backbone. Such non-enzymatic site-selective techniques require genetic engineering to place a biorthogonal functional group at the desired location in the protein and subsequently use that group for bioconjugation reaction. The engineered biorthogonal groups can be naturally occurring (e.g. cysteine) or completely artificial such as azide or carbonyl—ketone or aldehyde (FIG. 1). The latter is particularly attractive due to its highly electrophilic character and its ability to react with various nucleophilic groups in a specific manner, leading to the efficient preparation of homogeneous protein conjugates.

Aldehydes can be site-specifically incorporated into proteins chemically, enzymatically, and genetically. Chemical methods rely on the oxidation of @-amino alcohols in the form of N-terminal serine and threonine residues by sodium periodate (K. F. Geoghegan and J. G. Stroh, Bioconjugate Chem., 1992, 3, 138-146; O. EI-Mahdi and O. Melnyk, Bioconjugate Chem., 2013, 24, 735-765; Chen, W. et. al., Bioconjugate Chem., 2003, 14, 3, 614-618; T. R. Branson et. al., Angew. Chem., Int. Ed., 2014, 53, 8323-8327) and by the oxidation of N-terminal glycine with pyridoxal-5-phosphate (J. M. Gilmore et. al., Angew. Chem., Int. Ed., 2006, 45, 5307-5311; R. A. Scheck et. al., J. Am. Chem. Soc., 2008, 130, 11762-11770; L. S. Witus et. al., J. Am. Chem. Soc., 2010, 132, 16812-16817; M. Zhang et. al., Chin. J. Chem., 2011, 29, 1715-1720; L. S. Witus et. al., J. Am. Chem. Soc., 2013, 135, 17223-17229; M. Rashidian et. al., J. Am. Chem. Soc., 2013, 135, 16388-16396). Besides, post-translational enzymatic incorporation of aldehydes is achieved by including peptide recognition sequences of farnesyltransferase (C. Gauchet, et. al. J. Am. Chem. Soc., 2006, 128, 9274-9275; U. T. T. Nguyen et. al. ChemBioChem, 2007, 8, 408-423; M. Rashidian et. al. Chem. Commun., 2010, 46, 8998-9000; M. Rashidian et. al. J. Am. Chem. Soc., 2012, 134, 8455-8467), lipoic add ligase (J. D. Cohen et. al., ChemBioChem, 2012,13, 888-894; M. A. Gray et. al., ChemBioChem, 2016, 17, 155-158), and tubulin tyrosine ligase (D. Schumacher et. al., Angew. Chem., Int. Ed., 2015, 54, 13787-13791). In addition, site-directed mutagenesis of unnatural amino adds has been employed (L. Wang et. al., Proc. Natl. Acad. Sci. U.S.A, 2003, 100, 56-61; Y. Huang et. al., Bioorg. Med. Chem. Lett., 2010, 20, 878-880). Arguably, the most efficient, operationally simple and highly versatile method for genetic incorporation of aldehydes into proteins is the aldehyde tag approach, which takes an advantage of the co-translational recognition of the consensus sequence of the formylglycine-generating enzyme (FGE) (I. S. Carrico et. al. Nat. Chem. Biol., 2007, 3, 321-322). When proteins containing this consensus sequence (i.e., LCTPSR [SEQ ID NO:3]) are expressed in a cell line overexpressing human FGE, the enzyme converts the cysteine residue into a formylglycine residue co-translationally (FIG. 2). The resulting aldehyde-tagged protein is then harvested and purified by standard protein purification methods. This approach has been used to incorporate aldehyde functional handles into antibody constant regions for ligation by aldehyde-reactive linker-payloads to generate ADCs.

Because the genetic incorporation of the FGE recognition sequence is possible at virtually any location within the constant region of the antibody, this method is a truly versatile tool for placing multiple aldehyde tags at specific regions, which allows to rapidly screen conjugation sites and develop therapeutic ADC with more favorable biophysical properties.

There are a number of chemistries being used to conjugate aldehyde-tagged proteins with small molecules including oxime/hydrazone ligation (J. M. Gilmore et. al., Angew. Chem., Int. Ed., 2006, 45, 5307-5311; K. F. Geoghegan and J. G. Stroh, Bioconjugate Chem., 1992, 3, 138-146), Pictet-Spengler reaction (T. Sasaki et. al., Bioorg. Med. Chem. Lett., 2008, 18, 4550-4553), Mukaiyama aldol reaction (J. Alam et. al., J. Am. Chem. Soc., 2010, 132, 9546-9548), indium-mediated allylation (J. Alam et. al., Chem. Commun., 2011, 47, 9066-9068), Wittig reaction (M.-J. Ha et. al., Chem. Commun., 2012, 48, 11079-11081), and Knoevenagel condensation (R. Kudirka et. al., Chem. Biol., 2015, 22, 293-298), which all have been shown to provide access to homogeneous conjugates but have not risen to the status of practical and preferred approaches for making therapeutic ADCs. By contrast, Hydrazino-iso-Pictet-Spengler (HIPS) reaction (P. Agarwal et. al. Proc. Natl. Acad. Sci. U.S.A, 2013, 110, 46-51) has been used extensively with aldehyde-tagged antibodies to produce stable and highly efficacious ADCs (FIG. 3) (M. Bauzon et al., Oncolmmunology, 2019, 8, 4, e1565859; R. M. Barfield et al., Mol. Cancer Ther. 2020, 19, 1866-1874.), including one clinical candidate (P. Drake et al., Mol. Cancer Ther. 2018, 17, 161-168.).

### SUMMARY

The HIPS bioconjugation reaction features high efficiency and specificity under very mild conditions and results in the formation of stable carbon-carbon bond between antibody backbone and drug-linker. However, this method is not without limitations as it becomes increasingly challenging when multiple hydrophobic indole units are connected to the protein. Thus, for instance, having four aldehyde tags engaged in HIPS conjugation already affords conjugates with excessive hydrophobicity leading to a poorer PK profile and rendering such ADCs impractical. Therefore, the need for developing a conjugation method for aldehyde-tagged proteins that is efficient but does not incur excessive hydrophobicity of the resulting conjugates is desirable.

One particular reaction of aldehydes in this context is the formation of thiazolidine ring from 1,2-aminothiols (FIG. 4). Indeed, this transformation occurs under mild reaction conditions and affords a five-membered saturated nitrogen-containing heterocyclic linkage that is small and not particularly hydrophobic.

Surprisingly, this transformation has not been fully explored as a bioconjugation method, particularly with aldehyde-tagged proteins. It is likely that the lack of exploration is the result of early reports that have suggested the thiazolidine heterocycle is an unstable moiety, especially under physiologically relevant conditions, when the reversed reaction (ring opening) is favored (FIG. 4) (T. H. Fife et. al., J. Am. Chem. Soc. 1991, 113, 3071-3079; D. Bermejo-Velasco, et. al. Chem. Commun., 2018, 54, 12507 and references therein). The rare existing reports are limited to using an N-terminal cysteine group in the antibody to react with aldehyde-modified small molecules to form conjugates (FIG. 5). (G. Casi et. al. J. Am. Chem. Soc., 2012, 134, 5887; G. J. L. Bernardes et. al., Nat. Protoc., 2013, 8, 2079; Giulio et al. U.S. Pat. No. 9,198,979 B2; Dong et al. WO2007/139997). Thus, these previously reported instances have not attained the full potential of thiazolidine-based bioconjugation because: (a) the use of N-terminal cysteine has not allowed modifications to the 1,2-aminothiol counterpart which may lead to more efficient conjugation reaction and/or more stable conjugate; and (b) the conjugation site has been limited to the N-terminus of protein that leaves no options for optimizing the location of the drug within the conjugate to arrive at a construct with optimal biophysical properties.

Disclosed herein is a comprehensive and highly efficient method for generating therapeutic antibody-drug conjugates that utilizes genetically engineered aldehyde-tagged antibodies and bioconjugation reaction with 1,2-aminothiols to produce a stable thiazolidine link (FIG. 6).

In addition, the herein disclosed method of producing conjugates is fully compatible with the site-specific aldehyde tag approach to incorporating aldehyde functional groups into a protein (FIG. 2) and allows for the optimization of the resulting bioconjugates for desired properties. In particular, due to the small size of the thiazolidine linkage and its low hydrophobicity, it is possible to effectively utilize four, six, eight, or more aldehyde tags to generate ADCs with a high drug-to-antibody ratio without sacrificing pharmacokinetic properties. Overall, the present invention provides the use of noncanonical amino acids introduced to proteins along with simple biorthogonal reactions to create novel advantageous therapies.

The present disclosure provides thiazolidine (Tz) linkers for protein-drug conjugates. In addition, the disclosure also encompasses compounds useful for producing such protein-drug conjugates, as well as methods for production of such protein-drug conjugates. The disclosure also encompasses methods of using the protein-drug conjugates for the treatment of a disease or disorder in a subject.

Aspects of the present disclosure include a conjugate of formula (I):

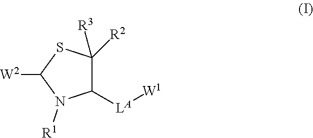

wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker;
W¹ is a drug; and
W² is a peptide.

In some embodiments, R¹ is hydrogen. In some embodiments, R¹ is alkyl.

In some embodiments, R² and R³ are each hydrogen. In some embodiments, one of R² and R³ is hydrogen and one of R² and R³ is alkyl. In some embodiments, R² and R³ are each alkyl.

In some embodiments, L^{A} comprises:

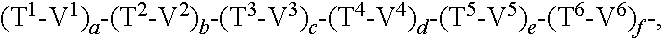

wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —CO—, —NR¹⁵, —NR¹⁵(CH₂)_{q}—, —NR¹⁵(C₆H₄)—, —CONR¹⁵—, —NR¹⁵CO—, —C(O)O—, —OC(O)—, —O—S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments,
(PEG)ₙ is

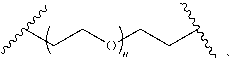

where n is an integer from 1 to 30;
EDA is an ethylene diamine moiety having the following structure:

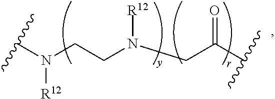

where y is an integer from 1 to 6 and r is 0 or 1;
4-amino-piperidine (4AP) is

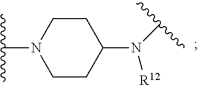

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

In some embodiments, one of T¹, T², T³, T⁴, T⁵, T⁶, V¹, V², V³, V⁴, V⁵ or V⁶ is a branched group.

In some embodiments, the branched group is selected from —CONR¹⁵— and 4AP.

In some embodiments, the branched group is attached to a compound of formula (II):

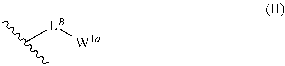

wherein:
L^{B} is a second linker; and
W^{1a} is a drug.

In some embodiments, L^{B} comprises:

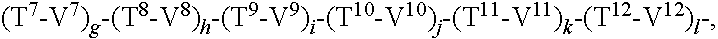

wherein
g, h, i, j, k and I are each independently 0 or 1;
T⁷, T⁸, T⁹, T¹⁰, T¹¹ and T¹² are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V⁷, V⁸, V⁹, V¹⁰, V¹¹ and V¹² are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, W¹ and W^{1a} are the same drug. In some embodiments, W¹ and W^{1a} are different drugs.

In some embodiments, the peptide comprises an antibody.

In some embodiments, the conjugate is selected from:

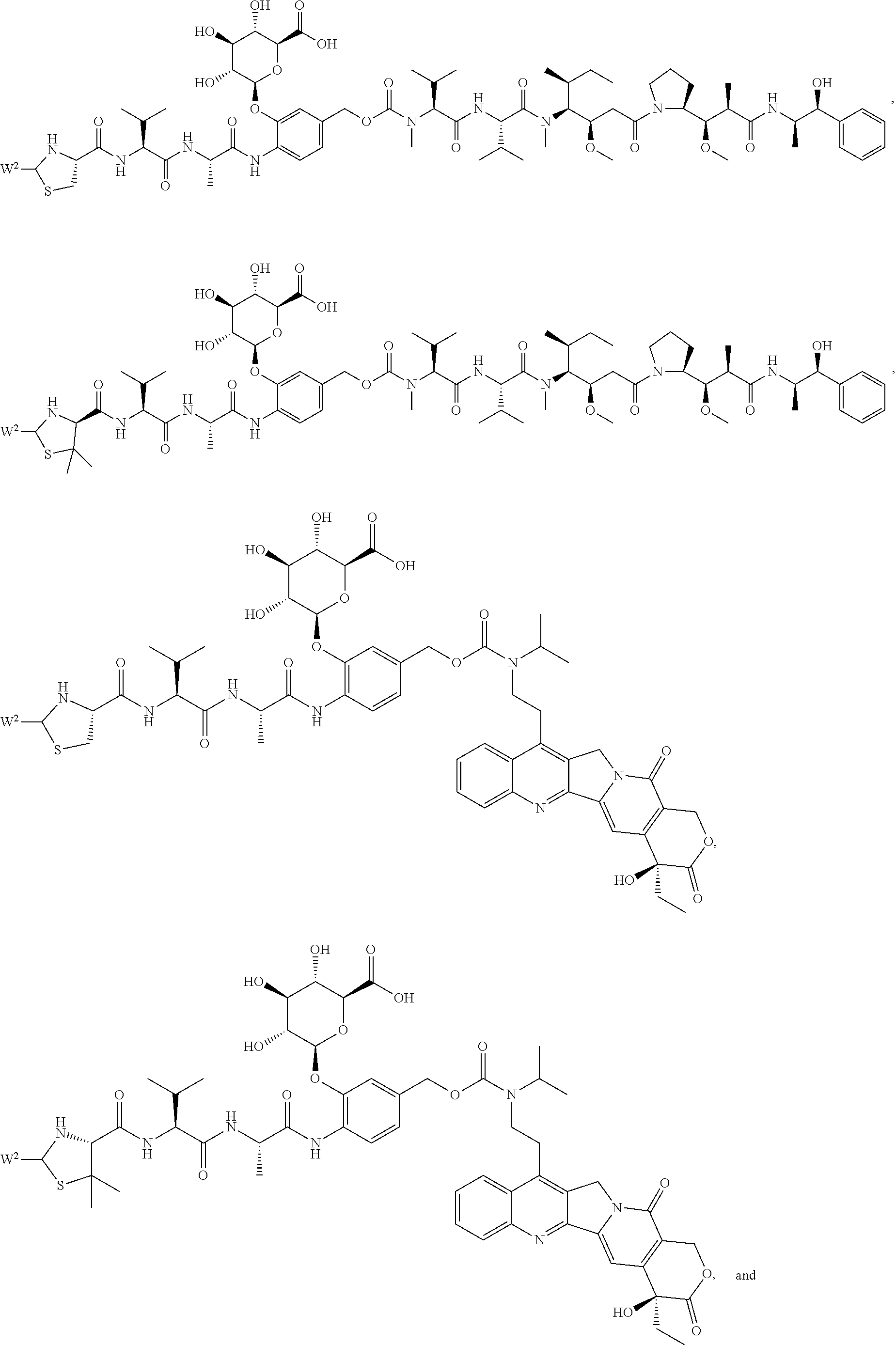

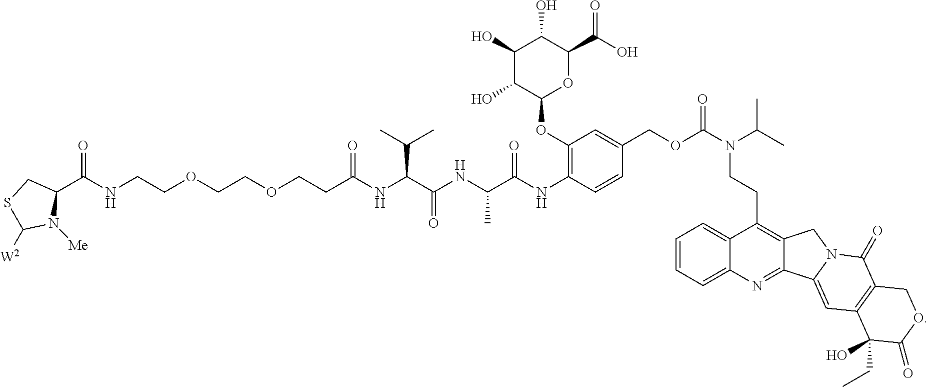

Aspects of the present disclosure include a pharmaceutical composition comprising: a conjugate of the present disclosure; and a pharmaceutically acceptable excipient.

Aspects of the present disclosure include a method comprising: administering to a subject a conjugate of the present disclosure.

Aspects of the present disclosure include a method of treating cancer in a subject, the method comprising: administering to the subject a therapeutically effective amount of a pharmaceutical composition comprising a conjugate of the present disclosure, wherein the administering is effective to treat cancer in the subject.

Aspects of the present disclosure include a method of producing a conjugate, the method comprising:
contacting an aldehyde-tagged peptide with a payload comprising a 1,2-aminothiol group under conditions to produce a conjugate of formula (I):

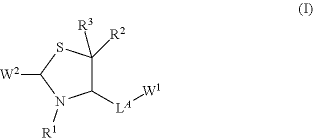

wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker;
W¹ is the payload; and
W² is the peptide.

### BRIEF DESCRIPTION OF THE DRAWINGS

FIG. 1 shows a scheme of the preparation of ADCs using unnatural or endogenous amino acids and major ADC components.

FIG. 2 shows a schematic diagram of generating aldehyde-tagged antibodies utilizing formylglycine generating enzyme (FGE) in CHO cells (SEQ ID Nos:1-2, from left to right).

FIG. 3 shows a schematic diagram of ADC preparation via HIPS conjugation of a double aldehyde-tagged antibody.

FIG. 4 shows a scheme of the formation of thiazolidine product from 1,2-aminothiol and aldehyde and its reversion at higher pH.

FIG. 5 shows a scheme of a thiazolidine conjugation using N-terminal cysteine of mAb and aldehyde-tagged drug-linker.

FIG. 6 shows a scheme of the conjugation of aldehyde-tagged antibody with 1,2-aminothiol drug-linker, according to embodiments of the present disclosure. Path A —productive path to form conjugate with gem-disubstituted substrate; path B—oxidative dimerization of unsubstituted drug-linker (unproductive path).

FIG. 7 shows a graph of the stability of thiol compound 12 in pH 5.5 buffer, according to embodiments of the present disclosure. HPLC trace at TO.

FIG. 8 shows a graph of the stability of thiol compound 12 in pH 5.5 buffe, according to embodiments of the present disclosure r. HPLC trace at day 7 at −20° C.

FIG. 9 shows a graph of the stability of thiol compound 12 in pH 5.5 buffer, according to embodiments of the present disclosure. HPLC on day 8 (incubated at RT for 24 hours).

FIG. 10 shows a graph of the stability of thiol compound 13 in pH 5.5 buffer, according to embodiments of the present disclosure. HPLC trace at TO.

FIG. 11 shows a graph of the stability of thiol compound 13 in pH 5.5 buffer, according to embodiments of the present disclosure. HPLC trace at day 7 at −20° C.

FIG. 12 shows a graph of the stability of thiol compound 13 in pH 5.5 buffer, according to embodiments of the present disclosure. HPLC on day 8 (incubated at RT for 24 hours).

FIG. 13 shows a graph of the stability of compound 12 conjugated to benzyloxyacetaldehyde in pH 5.5 buffer at RT, according to embodiments of the present disclosure. HPLC trace of conjugate 20 at TO.

FIG. 14 shows a graph of the stability of compound 13 conjugated to benzyloxyacetaldehyde in pH 5.5 buffer at RT, according to embodiments of the present disclosure. HPLC trace of conjugate 21 at TO.

FIG. 15 shows a graph of the stability of compound 12 conjugated to benzyloxyacetaldehyde in pH 5.5 buffer at RT, according to embodiments of the present disclosure. HPLC trace of conjugate 20 at day 7.

FIG. 16 shows a graph of the stability of compound 13 conjugated to benzyloxyacetaldehyde in pH 5.5 buffer at RT, according to embodiments of the present disclosure. HPLC trace of conjugate 21 at day 7.

FIG. 17 shows a graph of the conjugation of compound 19 with benzyloxyacetaldehyde in pH 5.5 buffer at RT to form conjugate 24, according to embodiments of the present disclosure. HPLC trace at TO.

FIG. 18 shows a graph of the conjugation of compound 19 with benzyloxyacetaldehyde in pH 5.5 buffer at RT to form conjugate 24, according to embodiments of the present disclosure. HPLC trace at 30 min.

FIG. 19 shows a graph of the conjugation of compound 19 with benzyloxyacetaldehyde in pH 5.5 buffer at RT to form conjugate 24, according to embodiments of the present disclosure. HPLC trace at 16 h.

FIG. 20 shows a graph of the stability of conjugate 20 in pH 5.5 buffer with excess formaldehyde present, according to embodiments of the present disclosure. HPLC trace of conjugate 20 after 24 h at RT.

FIG. 21 shows a graph of the stability of conjugate 21 in pH 5.5 buffer with excess formaldehyde present, according to embodiments of the present disclosure. HPLC trace of conjugate 21 after 24 h at RT.

FIG. 22 shows a graph of Compound 5 conjugated to CT-tagged HER2 mAb, which yields a DAR of 1.56 as determined by HIC, according to embodiments of the present disclosure.

FIG. 23 shows a graph of Conjugate 5-HER2/CT incubated in pH 7.4 buffer at 37° C. for 6 days, which shows a DAR of 1.03 as determined by HIC, according to embodiments of the present disclosure.

FIG. 24 shows a graph of Compound 8 conjugated to CT-tagged HER2 mAb, which yields a DAR of 1.62 as determined by HIC, according to embodiments of the present disclosure.

FIG. 25 shows a graph of Conjugate 8-HER2/CT incubated in pH 7.4 buffer at 37° C. for 6 days, which shows a DAR of 1.42 as determined by HIC, according to embodiments of the present disclosure.

FIG. 26 shows a graph of Compound 5 conjugated to CT-tagged FITC mAb, which yields a DAR of 1.23 as determined by HIC, according to embodiments of the present disclosure.

FIG. 27 shows a graph of Compound 8 conjugated to CT-tagged FITC mAb, which yields a DAR of 1.33 as determined by HIC, according to embodiments of the present disclosure.

FIG. 28 shows a graph of Compound 5 conjugated to CT-tagged CD33 mAb, which yields a DAR of 1.63 as determined by HIC, according to embodiments of the present disclosure.

FIG. 29 shows a graph of Conjugate 5-CD33/CT incubated in pH 5.5 buffer at 37° C. for 24 h, which shows a DAR of 1.55 as determined by HIC, according to embodiments of the present disclosure.

FIG. 30 shows a graph of Conjugate 5-CD33/CT incubated in pH 5.5 buffer at 37° C. for 72 h, which shows a DAR of 1.46 as determined by HIC, according to embodiments of the present disclosure.

FIG. 31 shows a graph of Compound 8 conjugated to CT-tagged CD33 mAb, which yields a DAR of 1.80 as determined by HIC, according to embodiments of the present disclosure.

FIG. 32 shows a graph of Conjugate 8-CD33/CT incubated in pH 5.5 buffer at 37° C. for 24 h, which shows a DAR of 1.79 as determined by HIC, according to embodiments of the present disclosure.

FIG. 33 shows a graph of Conjugate 8-CD33/CT incubated in pH 5.5 buffer at 37° C. for 72 h, which shows a DAR of 1.78 as determined by HIC, according to embodiments of the present disclosure.

FIG. 34 shows a graph of Compound 13 conjugated to CH1-tagged FITC mAb following the unoptimized procedure, which yields a DAR of 1.30 as determined by PLRP, according to embodiments of the present disclosure.

FIG. 35 shows a graph of Compound 13 conjugated to CH1-tagged FITC mAb following the optimized procedure, which yields a DAR of 1.80 as determined by PLRP, according to embodiments of the present disclosure.

FIG. 36 shows a graph of Compound 13 conjugated to CH1/CT-tagged HER2 mAb following the unoptimized procedure, which yields a DAR of 1.32 as determined by PLRP, according to embodiments of the present disclosure.

FIG. 37 shows a graph of Compound 13 conjugated to CH1/CT-tagged HER2 mAb following the optimized procedure, which yields a DAR of 2.81 as determined by PLRP, according to embodiments of the present disclosure.

FIG. 38 shows a graph of Compound 13 conjugated to CH1-tagged FITC mAb following the optimized procedure, which yields a DAR of 1.73 as determined by PLRP, according to embodiments of the present disclosure.

FIG. 39 shows a graph of Acetonide compound 14 conjugated to CH1-tagged FITC mAb, which yields the 12-FITC/CH1 conjugate with DAR of 1.8 as determined by PLRP, according to embodiments of the present disclosure.

FIG. 40 shows a graph of the rat serum stability data 8-HER2/CH1 and 13-HER2/CH1 conjugates showing remaining concentrations of intact ADC as measured by ELISA in samples incubated at 37° C. and aliquoted at the times shown, according to embodiments of the present disclosure.

FIG. 41 shows a graph of the in vitro potency of control molecules or anti-HER2 ADCs conjugated to Compound 5 against HER2+ NCI-N87 cells, according to embodiments of the present disclosure.

FIG. 42 shows a graph of the in vitro potency of control molecules or anti-HER2 ADCs conjugated to Compound 5 against HER2+ SKBR3 cells, according to embodiments of the present disclosure.

FIG. 43 shows a graph of the in vitro potency of control molecules or anti-HER2 ADCs conjugated to Compound 8 against HER2+ NCI-N87 cells, according to embodiments of the present disclosure.

FIG. 44 shows a graph of the in vitro potency of control molecules or anti-HER2 ADCs conjugated to Compound 8 against HER2+ NCI-N87 cells, according to embodiments of the present disclosure.

FIG. 45 shows schematic drawings of ELISA assay methods used to determine total antibody (left panel) and total ADC (right panel).

FIG. 46 shows a graph of an in vivo pharmacokinetic study in rats of a Compound 8-HER2/CH1 conjugate as measured by ELISA, according to embodiments of the present disclosure.

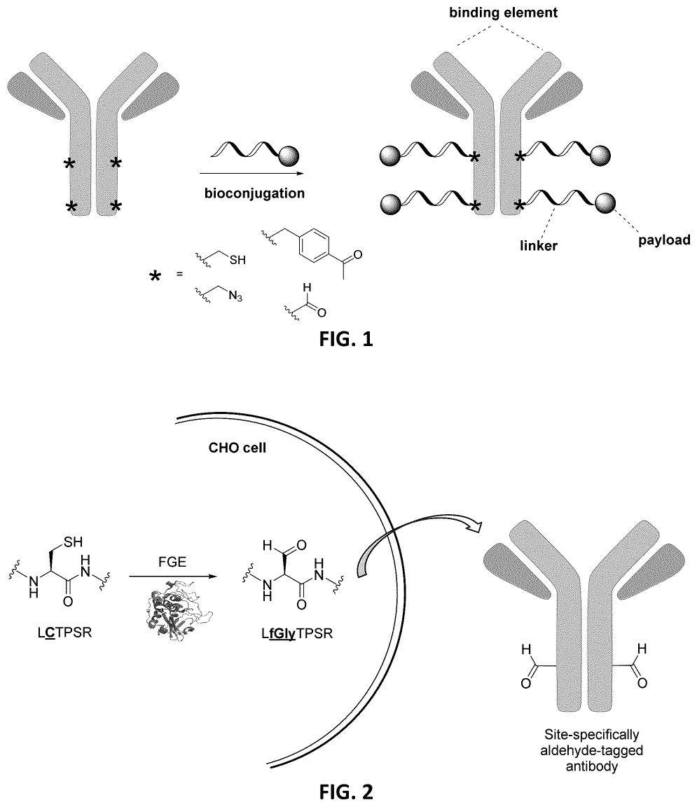

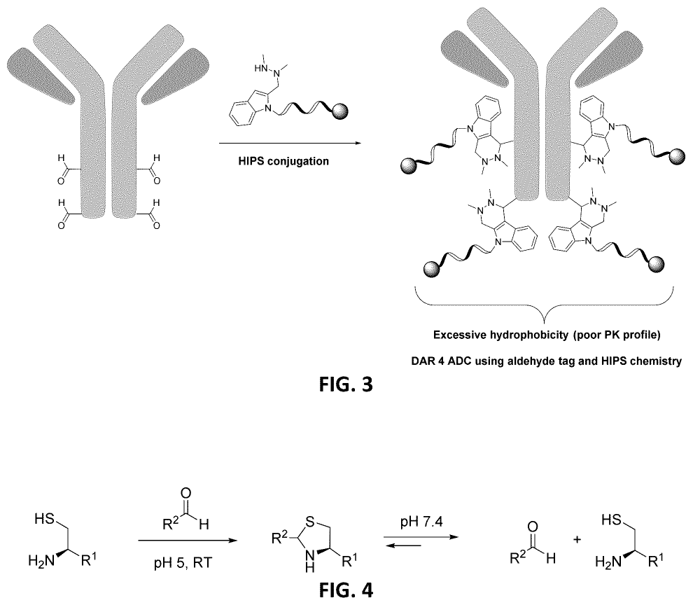

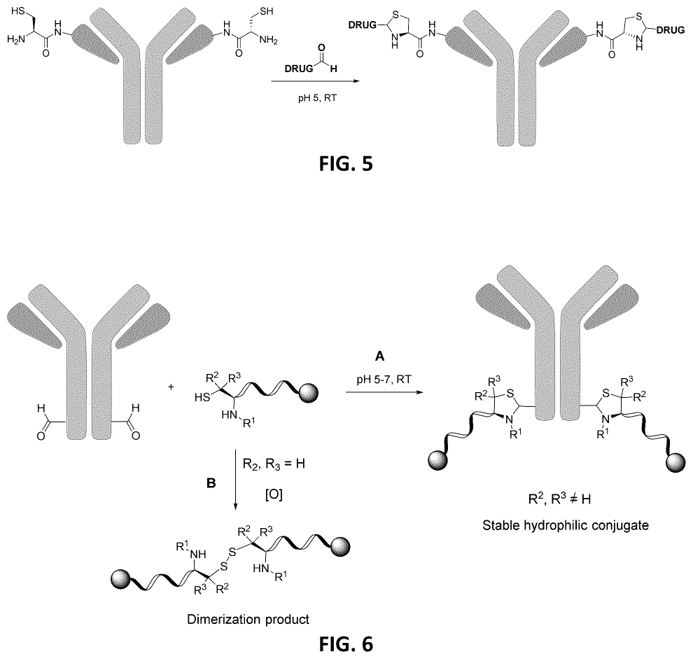

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl(CH₃—), ethyl(CH₃CH₂—), n-propyl(CH₃CH₂CH₂—), isopropyl((CH₃)₂CH—), n-butyl(CH₃CH₂CH₂CH₂—), isobutyl((CH₃)₂CHCH₂—), sec-butyl((CH₃)(CH₃CH₂)CH—), t-butyl((CH₃)₃C—), n-pentyl(CH₃CH₂CH₂CH₂CH₂—), and neopentyl((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), -NR- (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰- and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O-, cycloalkyl-O-, cycloalkenyl-O-, and alkynyl-O-, where alkenyl, cycloalkyl, cycloalkenyl, and alkynyl are as defined herein.

The term “substituted alkoxy” refers to the groups substituted alkyl-O-, substituted alkenyl-O-, substituted cycloalkyl-O-, substituted cycloalkenyl-O-, and substituted alkynyl-O-where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O- wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂— heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C—CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂— heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O) substituted alkyl, N R²⁰C(O)cycloalkyl, —NR²⁰C(O) substituted cycloalkyl, —NR²⁰C(O)cycloalkenyl, —NR²⁰C(O) substituted cycloalkenyl, —NR²⁰C(O)alkenyl, —NR²⁰C(O) substituted alkenyl, —NR²⁰C(O)alkynyl, —NR²⁰C(O) substituted alkynyl, —NR²⁰C(O)aryl, —NR²⁰C(O) substituted aryl, —NR²⁰C(O)heteroaryl, —NR²⁰C(O) substituted heteroaryl, —NR²⁰C(O)heterocyclic, and —NR²⁰C(O) substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group -N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O— alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂— heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N—)O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl and —SO₂— heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group —SO₂-alkyl, —SO₂-substituted alkyl, —SO₂-alkenyl, —SO₂— substituted alkenyl, —SO₂-cycloalkyl, —SO₂-substituted cycloalkyl, —SO₂-cycloalkenyl, —SO₂— substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂— substituted heteroaryl, —OSO₂-heterocyclic, and —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfate” or “sulfate ester” refers the group —O—SO₂—OH, —O—SO₂—O-alkyl, —O—SO₂—O—substituted alkyl, —O—SO₂—O-alkenyl, —O—SO₂—O-substituted alkenyl, —O—SO₂—O-cycloalkyl, —O—SO₂— O-substituted cycloalkyl, —O—SO₂—O-cycloalkenyl, —O—SO₂—O-substituted cylcoalkenyl, —O—SO₂—O—aryl, —O—SO₂—O-substituted aryl, —O—SO₂—O-heteroaryl, —O—SO₂—O-substituted heteroaryl, —O—SO₂— O-heterocyclic, and —O—SO₂—O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S- wherein the aryl group is as defined herein including optionally substituted aryl groups also defined herein.

The term “thioheteroaryloxy” refers to the group heteroaryl-S- wherein the heteroaryl group is as defined herein including optionally substituted aryl groups as also defined herein.

The term “thioheterocyclooxy” refers to the group heterocyclyl-S- wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸OR⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have—H or C—C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂M⁺, —CO₂R⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OCO₂M⁺, —OCO₂R⁷⁰, —OC(S)OR⁷⁰, —NR⁷⁰C(O)R⁷⁰, —NR⁷⁰C(S)R⁷⁰, —NR⁷⁰CO₂M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸OR⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Patient” refers to human and non-human subjects, especially mammalian subjects.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In certain embodiments, a polypeptide is an antibody.

“Native amino acid sequence” or “parent amino acid sequence” are used interchangeably herein to refer to the amino acid sequence of a polypeptide prior to modification to include at least one modified amino acid residue.

“Native amino acid” or “native amino acid residue” are used interchangeably herein to refer to natural amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). A native amino acid can be positioned in its naturally occurring position in a native amino acid sequence.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

The term “glycoside” or “glycosyl” refers to a sugar molecule or group bound to a moiety via a glycosidic bond. For example, the moiety that the glycoside is bound to can be a cleavable linker as described herein. A glycosidic bond can link the glycoside to the other moiety through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some cases, glycosides can be cleaved from the moiety they are attached to, such as by chemically-mediated hydrolysis or enzymatically-mediated hydrolysis.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies, chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). In certain embodiments, framework substitutions are identified by modeling of the interactions of the CDR and framework residues to identify framework residues important for antigen binding and sequence comparison to identify unusual framework residues at particular positions (see, e.g., U.S. Pat. No. 5,585,089; Riechmann et al., Nature 332:323 (1988)). Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, and PCT publications WO 98/45331 and WO 98/45332. In particular embodiments, a subject rabbit antibody may be humanized according to the methods set forth in US20040086979 and US20050033031. Accordingly, the antibodies described above may be humanized using methods that are well known in the art.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

An immunoglobulin polypeptide immunoglobulin light or heavy chain variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, that is the combined framework regions of the constituent light and heavy chains, serves to position and align the CDRs. The CDRs are primarily responsible for binding to an epitope of an antigen.

A “parent Ig polypeptide” is a polypeptide comprising an amino acid sequence which lacks an aldehyde-tagged constant region as described herein. The parent polypeptide may comprise a native sequence constant region, or may comprise a constant region with pre-existing amino acid sequence modifications (such as additions, deletions and/or substitutions).

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (fGly) in lieu of cysteine or serine in the motif. Other exemplary reactive partners include an aldehyde of an fGly residue of a converted aldehyde tag (e.g., a reactive aldehyde group) and an “aldehyde-reactive reactive partner,” which comprises an aldehyde-reactive group and a moiety of interest, and which reacts to form a reaction product of a polypeptide having the moiety of interest conjugated to the polypeptide through the fGly residue. For example, a 1,2-aminothiol group as described herein can be an aldehyde-reactive group, which reacts with an aldehyde tag containing a formylglycine (fGly) to produce a thiazolidine (Tz) conjugation moiety as described herein.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides thiazolidine (Tz) linkers for protein-drug conjugates. In addition, the disclosure also encompasses compounds useful for producing such protein-drug conjugates, as well as methods for production of such protein-drug conjugates. The disclosure also encompasses methods of using the protein-drug conjugates for the treatment of a disease or disorder in a subject.

### Conjugates

The present disclosure provides a protein conjugate. By “conjugate” is meant a polypeptide (e.g., protein or peptide) that is covalently attached to one or more other moieties (e.g., drugs or active agents). For example, a protein conjugate according to the present disclosure includes one or more drugs or active agents covalently attached to a protein. In certain embodiments, the polypeptide (e.g., protein or peptide) and the one or more moieties are bound to each other through one or more functional groups and covalent bonds. For example, the one or more functional groups and covalent bonds can include a thiazolidine (Tz) linker as described herein.

In certain embodiments, the conjugate is an antibody-drug conjugate (ADC). For example, the protein in the protein conjugate can be an antibody. In some cases, a polypeptide (e.g., a protein or an antibody) is covalently attached to one or more other moieties (e.g., drugs or active agents). For example, an antibody-drug conjugate according to the present disclosure includes one or more drugs or active agents covalently attached to an antibody. In certain embodiments, the antibody and the one or more drugs or active agents are bound to each other through one or more functional groups and covalent bonds. For example, the one or more functional groups and covalent bonds can include a thiazolidine linker as described herein. In certain embodiments, the one or more moieties in the conjugate can be referred to herein as a “payload”.

In certain embodiments, the conjugate is a protein conjugate, which includes a polypeptide (e.g., an antibody) conjugated to one or more other moieties. In certain embodiments, the one or more moieties conjugated to the polypeptide can each independently be any of a variety of moieties of interest such as, but not limited to, a drug, an active agent, a detectable label, a water-soluble polymer, or a moiety for immobilization of the polypeptide to a membrane or a surface. In certain embodiments, the conjugate is a drug conjugate, where the polypeptide is an antibody, thus providing an antibody-drug conjugate (ADC). For instance, the conjugate can be a drug conjugate, where a polypeptide is conjugated to one or more drugs or active agents.

The one or more drugs or active agents can be conjugated to the polypeptide (e.g., antibody) at any desired site of the polypeptide. Thus, the present disclosure provides, for example, a polypeptide having a drug or active agent conjugated at a site at or near the C-terminus of the polypeptide. Other examples include a polypeptide having a drug or active agent conjugated at a position at or near the N-terminus of the polypeptide. Examples also include a polypeptide having a drug or active agent conjugated at a position between the C-terminus and the N-terminus of the polypeptide (e.g., at an internal site of the polypeptide). Combinations of the above are also possible where the polypeptide is conjugated to two or more drugs or active agents.

In certain embodiments, a conjugate of the present disclosure includes one or more drugs or active agents conjugated to an amino acid residue of a polypeptide at the α-carbon of an amino acid residue. Stated another way, a conjugate includes a polypeptide where the side chain of one or more amino acid residues in the polypeptide has been modified and attached to one or more drugs or active agents (e.g., attached to one or more drugs or active agents through a linker as described herein). For example, a conjugate includes a polypeptide where the α-carbon of one or more amino acid residues in the polypeptide has been modified and attached to one or more drugs or active agents (e.g., attached to one or more drugs or active agents through a linker as described herein).

Embodiments of the present disclosure include conjugates where a polypeptide is conjugated to one or more moieties, such as 2 moieties, 3 moieties, 4 moieties, 5 moieties, 6 moieties, 7 moieties, 8 moieties, 9 moieties, 10 moieties, 11 moieties, 12 moieties, 13 moieties, 14 moieties, 15 moieties, 16 moieties, 17 moieties, 18 moieties, 19 moieties, or 20 or more moieties. The moieties may be conjugated to the polypeptide at one or more sites in the polypeptide. For example, one or more moieties may be conjugated to a single amino acid residue of the polypeptide. In some cases, one moiety is conjugated to an amino acid residue of the polypeptide. In other embodiments, two moieties may be conjugated to the same amino acid residue of the polypeptide. In other embodiments, a first moiety is conjugated to a first amino acid residue of the polypeptide and a second moiety is conjugated to a second amino acid residue of the polypeptide. Combinations of the above are also possible, for example where a polypeptide is conjugated to a first moiety at a first amino acid residue and conjugated to two other moieties at a second amino acid residue. Other combinations are also possible, such as, but not limited to, a polypeptide conjugated to first and second moieties at a first amino acid residue and conjugated to third and fourth moieties at a second amino acid residue, etc. In some cases, two or more amino acid residues in the polypeptide are each conjugated to a pair of moieties (i.e., two moieties), where each pair of moieties is conjugated to the polypeptide through a branched linker as described herein. In some cases, one amino acid residue in the polypeptide is conjugated to a pair of moieties through a branched linker as described herein. In some cases, two amino acid residues in the polypeptide are each conjugated to a pair of moieties through a branched linker as described herein. In some cases, three amino acid residues in the polypeptide are each conjugated to a pair of moieties through a branched linker as described herein. In some cases, four amino acid residues in the polypeptide are each conjugated to a pair of moieties through a branched linker as described herein. In some cases, five amino acid residues in the polypeptide are each conjugated to a pair of moieties through a branched linker as described herein. In some cases, six amino acid residues in the polypeptide are each conjugated to a pair of moieties through a branched linker as described herein. In some cases, seven amino acid residues in the polypeptide are each conjugated to a pair of moieties through a branched linker as described herein. In some cases, eight amino acid residues in the polypeptide are each conjugated to a pair of moieties through a branched linker as described herein.

The one or more amino acid residues of the polypeptide that are conjugated to the one or more moieties may be naturally occurring amino acids, unnatural amino acids, or combinations thereof. For instance, the conjugate may include one or more drugs or active agents conjugated to one or more naturally occurring amino acid residues (e.g., one or more native amino acid residues) of the polypeptide. In other instances, the conjugate may include one or more drugs or active agents conjugated to one or more unnatural amino acid residues of the polypeptide. One or more drugs or active agents may be conjugated to the polypeptide at a single natural (e.g., native) or unnatural amino acid residue as described above. One or more natural (e.g., native) or unnatural amino acid residues in the polypeptide may be conjugated to the moiety or moieties as described herein. For example, two (or more) amino acid residues (e.g., natural (e.g., native) or unnatural amino acid residues) in the polypeptide may each be conjugated to one or two moieties, such that multiple sites in the polypeptide are conjugated to the moieties of interest.

In certain embodiments, the polypeptide (e.g., antibody) and the moiety of interest (e.g., drug or active agent) are conjugated through a conjugation moiety. For example, the polypeptide and the moiety of interest may each be bound (e.g., covalently bonded) to the conjugation moiety, thus indirectly binding the polypeptide and the moiety of interest together through the conjugation moiety.

In some cases, the conjugation moiety includes a thiazolidine moiety, or a derivative of a thiazolidine moiety. The conjugation moiety may also include or be produced from a precursor to a thiazolidine moiety or derivative thereof. For example, the precursor to a thiazolidine moiety may include a 1,2-aminothiol group or derivative thereof. In some instances, reaction of a 1,2-aminothiol group with an aldehyde group can produce the thiazolidine conjugation moiety or derivative thereof. For instance, a thiazolidine conjugation moiety may be produced by conjugation of a 1,2-aminothiol group to a formylglycine residue of the polypeptide (e.g., antibody), such as in an aldehyde-tagged polypeptide (e.g., antibody), thus indirectly binding the polypeptide (e.g., antibody) and the moiety of interest (e.g., drug or active agent) together through the thiazolidine conjugation moiety.

In certain embodiments, the polypeptide may be conjugated to one or more moieties of interest, where one or more amino acids of the polypeptide are modified before conjugation to the one or more moieties of interest. Modification of one or more amino acids of the polypeptide may produce a polypeptide that contains one or more reactive groups suitable for conjugation to the one or more moieties of interest. In some cases, the polypeptide may include one or more modified amino acid residues to provide one or more reactive groups suitable for conjugation to the one or more moieties of interest. For example, an amino acid of the polypeptide may be modified to include a reactive aldehyde group (e.g., a reactive aldehyde). A reactive aldehyde may be included in an “aldehyde tag” or “ald-tag”, which, as used herein, refers to an amino acid sequence derived from a sulfatase motif (e.g., L(C/S)TPSR [SEQ ID NO:1]) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of an FGE, e.g., L(fGly)TPSR [SEQ ID NO:2]). A converted sulfatase motif may be produced from an amino acid sequence that includes an “unconverted” sulfatase motif (i.e., a sulfatase motif in which the cysteine or serine residue has not been converted to fGly by an FGE, but is capable of being converted, e.g., an unconverted sulfatase motif with the sequence: L(C/S)TPSR [SEQ ID NO:1]). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly).

In certain embodiments, the polypeptide (e.g., antibody) may be modified to include the fGly or aldehyde tag at any desired site of the polypeptide (e.g., antibody). Thus, the present disclosure provides, for example, a polypeptide (e.g., antibody) that has been site-specifically modified to include an fGly or aldehyde tag at a site at or near the C-terminus of the polypeptide. Other examples include a polypeptide (e.g., antibody) that has been site-specifically modified to include an fGly or aldehyde tag at a position at or near the N-terminus of the polypeptide. Examples also include a polypeptide (e.g., antibody) that has been site-specifically modified to include an fGly or aldehyde tag at a position between the C-terminus and the N-terminus of the polypeptide (e.g., at an internal site of the polypeptide). Combinations of the above are also possible where the polypeptide (e.g., antibody) has been site-specifically modified to include an fGly or aldehyde tag at two or more specific positions. Accordingly, embodiments of the polypeptide (e.g., antibody) include those where the polypeptide (e.g., antibody) includes one or more site-specifically engineered conjugation sites, such as an fGly or aldehyde tag as described herein. Each site-specifically engineered conjugation site may be conjugated to one or more drugs or active agents through a linker as described herein.

Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the polypeptide containing the fGly residue may be conjugated to the one or more moieties of interest by reaction of the fGly with a compound (e.g., a compound containing a 1,2-aminothiol group, as described herein). For example, an fGly-containing polypeptide may be contacted with a reactive partner under conditions suitable to provide for conjugation of one or more drugs or active agents to the polypeptide. In some instances, the reactive partner may include a 1,2-aminothiol group as described herein. For example, one or more drugs or active agents may be attached to a 1,2-aminothiol group. In some cases, the one or more drugs or active agents are attached to a 1,2-aminothiol group, such as covalently attached to a 1,2-aminothiol group, where each drug or active agent is attached through a corresponding linker to the 1,2-aminothiol group.

In certain embodiments, a conjugate of the present disclosure includes a polypeptide (e.g., an antibody) having at least one amino acid residue that has been attached to one or more moieties of interest (e.g., one or more drugs or active agents). In order to make the conjugate, an amino acid residue of the polypeptide may be modified and then coupled to one or more drugs or active agents attached to a 1,2-aminothiol group as described herein. In certain embodiments, an amino acid residue of the polypeptide (e.g., antibody) is a cysteine or serine residue that is converted to an fGly residue, as described above. In certain embodiments, the converted amino acid residue (e.g., fGly residue) is conjugated to one or more drugs or active agents containing 1,2-aminothiol group as described herein to provide a conjugate of the present disclosure where the one or more drugs or active agents are conjugated to the polypeptide through a thiazolidine conjugation moiety, which was formed via reaction of the 1,2-aminothiol group with the fGly residue. As used herein, the term fGly′ refers to the amino acid residue of the polypeptide (e.g., antibody) that is coupled to the one or more moieties of interest (e.g., one or more drugs or active agents).

In certain embodiments, the conjugate includes a polypeptide (e.g., an antibody) having at least one amino acid residue attached to a thiazolidine conjugation moiety as described herein, which in turn is attached to one or more drugs or active agents through one or more corresponding linkers. For instance, the conjugate may include a polypeptide (e.g., an antibody) having at least one amino acid residue (fGly′) that is conjugated to the one or more moieties of interest (e.g., one or more drugs or active agents) as described above.

In certain embodiments, the conjugation moiety may be attached (e.g., covalently attached) to a linker. As such, embodiments of the present disclosure include a conjugation moiety attached to a drug or active agent through a linker. Various embodiments of the linkers that may be used to couple the conjugation moiety to the drug or active agent are described in detail herein. For example, in some instances, the linker is a thiazolidine linker as described herein. In some instances, the linker is a cleavable linker, such as a cleavable linker as described herein.

In certain embodiments, the linker is a branched linker, such as a branched linker as described herein. In some instances, the conjugation moieties may be attached (e.g., covalently attached) to two or more linkers. As such, embodiments of the present disclosure include a conjugation moiety attached to two or more drugs or active agents each through a corresponding linker. Thus, conjugates of the present disclosure may include two or more linkers, where each linker attaches a corresponding drug or active agent to the conjugation moiety. Accordingly, the conjugation moiety and two or more linkers may be viewed overall as a “branched linker”, where the conjugation moiety is attached to two or more “branches”, where each branch includes a linker attached to a drug or active agent.

In some cases, to produce the conjugate, the polypeptide may be conjugated to the moiety of interest by reaction of an amino acid residue of the polypeptide with a compound (e.g., a compound containing a conjugation moiety, as described herein). For example, a polypeptide may be contacted with a reactive partner-containing drug under conditions suitable to provide for conjugation of the drug to the polypeptide. In some instances, the reactive partner-containing drug may include a conjugation moiety as described above. For example, a drug or active agent may be modified to include a conjugation moiety. In some cases, the drug or active agent is attached to a conjugation moiety, such as covalently attached to conjugation moiety through a linker, such as a linker as described in detail herein.

In certain embodiments, a conjugate of the present disclosure includes a polypeptide (e.g., an antibody) having at least one amino acid residue that has been attached to one or more moieties of interest (e.g., drugs or active agents). In order to make the conjugate, an amino acid residue of the polypeptide may be coupled to one or more drugs or active agents attached to a conjugation moiety as described above. In certain embodiments, the amino acid residue is conjugated to a drug or active agent containing a conjugation moiety as described above to provide a conjugate of the present disclosure where the one or more drugs or active agents are conjugated to the polypeptide through the conjugation moiety.

In certain embodiments, the conjugation moiety includes a 1,2-aminothiol group, or a derivative of a 1,2-aminothiol group. For instance, a general scheme for coupling a moiety of interest to a polypeptide through a thiazolidine conjugation moiety is shown in the general reaction scheme in FIG. 6, path A. In the reaction scheme shown in FIG. 6, path A, the moiety of interest (e.g., a drug or active agent) is represented by the shaded sphere. As shown in the reaction scheme, a polypeptide (antibody) that includes a 2-formylglycine residue (fGly) is reacted with a drug or active agent that has been modified to include a 1,2-aminothiol group to produce a polypeptide conjugate attached through the thiazolidine conjugation moiety, thus attaching the drug or active agent to the polypeptide (antibody) through the thiazolidine conjugation moiety.

In addition, as shown in the reaction scheme, the thiazolidine conjugation moiety may be attached (e.g., covalently attached) to a linker, which is represented by the wavy line. As such, embodiments of the present disclosure include a thiazolidine conjugation moiety attached to a drug or active agent through a linker. Various embodiments of the linker that may couple the thiazolidine conjugation moiety to the drug or active agent are described in detail herein.

In certain embodiments, the conjugate includes a polypeptide (e.g., an antibody) having at least one amino acid residue attached to a linker as described herein, which in turn is attached to one or more drugs or active agents. For instance, the conjugate may include a polypeptide (e.g., an antibody) having at least one amino acid residue (fGly′) that is conjugated to the one or more moieties of interest (e.g., one or more drugs or active agents) as described herein. For example, in some instances, the conjugate includes one or more of a first drug attached to the antibody at a site-specifically engineered conjugation site of the antibody through a first linker, and one or more of a second drug attached to the antibody at a site-specifically engineered conjugation site of the antibody through a second linker.

Embodiments of the present disclosure include conjugates where an antibody is conjugated to one or more drug moieties, such as two or more drug moieties, such as 3 drug moieties, 4 drug moieties, 5 drug moieties, 6 drug moieties, 7 drug moieties, 8 drug moieties, 9 drug moieties, 10 drug moieties, 11 drug moieties, 12 drug moieties, 13 drug moieties, 14 drug moieties, 15 drug moieties, 16 drug moieties, 17 drug moieties, 18 drug moieties, 19 drug moieties, or 20 or more drug moieties. The drug moieties may be conjugated to the antibody at one or more sites in the antibody, as described herein. In certain embodiments, the conjugates have an average drug-to-antibody ratio (DAR) (molar ratio) in the range of from 0.1 to 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 10, such as 1, 2, 3, 4, 5, 6, 7, 8, 9, or 10. In certain embodiments, the conjugates have an average DAR from 10 to 20, such as 10, 11, 12, 13, 14, 15, 16, 17, 18, 19, or 20. In certain embodiments, the conjugates have an average DAR of 1 to 10. In certain embodiments, the conjugates have an average DAR of 1 to 5 (e.g., 4). In certain embodiments, the conjugates have an average DAR of 5 to 10 (e.g., 8).). In certain embodiments, the conjugates have an average DAR of 8 to 12 (e.g., 10). In certain embodiments, the conjugates have an average DAR of 10 to 15 (e.g., 12). In certain embodiments, the conjugates have an average DAR of 15 to 20 (e.g., 16). In certain embodiments, the conjugates have an average DAR ranging from 2 to 20, such as 4 to 16. By average is meant the arithmetic mean.

As indicated above, embodiments of the conjugates of the present disclosure include conjugates attached to one or more of a first drug and one or more of a second drug. In some cases, DAR refers to the total DAR of the conjugate (e.g., the average number of the first drug plus the second drug linked to each antibody). In some cases, DAR refers to the DAR of one of the drugs attached to the antibody (e.g., the average number of the first drug linked to each antibody, or the average number of the second drug linked to each antibody). In some instances, the total DAR of the conjugate is the DAR of the first drug plus the DAR of the second drug.

Combinations of the same or different payloads (e.g., drugs or active agents) may be conjugated to the antibody through the first linker and the second linker. In certain embodiments, the first drug attached to the first linker and the second drug attached to the second linker are the same. In other embodiments, the first drug attached to the first linker and the second drug attached to the second linker are different drugs or active agents.

In some embodiments, where two different drugs or active agents are attached to the first and second linkers, the drugs or active agents may be selected from drugs and active agents that have a synergistic therapeutic effect. By “synergistic”, “synergism” or “synergy” is meant a therapeutic effect that is greater than the sum of the effects of the drugs or active agents taken separately. For example, in some instances, the use of two different drugs or active agents attached to the first and second linkers may provide a lower therapeutically effective concentration at which both payloads act, thereby increasing overall potency of the ADC.

In some embodiments, where two different drugs or active agents are attached to the first and second linkers, the drugs or active agents may be selected from drugs and active agents that provide an enhanced therapeutic benefit as compared to the use of the drugs or active agents separately. For example, the drugs or active agents may provide an increased effect on drug delivery of the ADC (e.g., some payloads, such as the iRGD peptide, can increase extravasation into tissues and augment tumor penetration).

In some embodiments, where two different drugs or active agents are attached to the first and second linkers, the drugs or active agents may be selected from drugs and active agents that use different mechanisms of action. In some cases, this may provide a decrease in tumor drug resistance by targeting multiple pathways. Examples of payload combinations can include, but are not limited to, cytotoxic drugs, immunomodulatory molecules to activate or inhibit immune cell populations, cytokines, hormones, chelating agents loaded with radioisotopes, and the like.

In some embodiments, where two different payloads are attached to the first and second linkers, the payloads may be selected from combinations of drugs or active agents and detectable labels. For example, a first payload may be a detectable label that is used as an imaging agent or tracer to detect the location of the ADC in vivo, while a second payload may be a drug or active agent that provides a therapeutic activity.

In certain embodiments, the conjugate is a conjugate of formula (I):

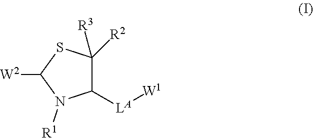

wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker;
W¹ is a drug; and
W² is a peptide.

The substituents related to conjugates of formula (I) are described in more detail below.

In certain embodiments, R¹ is selected from hydrogen, alkyl, substituted alkyl, alkenyl, substituted alkenyl, alkynyl, and substituted alkynyl. In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is methyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl.

In certain embodiments, R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl.

In certain embodiments, R² is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are the same. In certain embodiments, R² and R³ are each hydrogen. In certain embodiments, R² and R³ are each alkyl. In certain embodiments, R² and R³ are each methyl.

In certain embodiments, R² and R³ are different. In certain embodiments, one of R² and R³ is hydrogen and one of R² and R³ is alkyl. For example, in some instances, R² is hydrogen and R³ is alkyl (e.g., methyl). In other embodiments, R² is alkyl (e.g., methyl) and R³ is hydrogen.

In certain embodiments, W¹ is a drug (or active agent). Further description of drugs (or active agents) that find use in the subject conjugates is found in the disclosure herein.

In certain embodiments, W² is a peptide (e.g., polypeptide or protein). For example, W² can be an antibody. In certain embodiments, W² comprises one or more amino acid residues that are attached to the drug, W¹, through the linker (e.g., L^{A} and/or L^{B}), as described herein. In certain embodiments, the polypeptide (e.g., antibody) is attached to the rest of the conjugate through one or more amino acid residues as described herein. Further description of polypeptides and antibodies that find use in the subject conjugates is found in the disclosure herein.

In certain embodiments, L^{A} is a linker (e.g., a first linker). The linker, L^{A}, may be attached on one end of the linker to the drug, W¹, and attached at another end of the linker to the polypeptide (e.g., antibody), W². Linkers suitable for L^{A} are described in more detail below.

In certain embodiments, the conjugate of formula (I) includes a linker, L^{A}. The linker may be utilized to bind one or more moieties of interest (e.g., drug or active agent) to one or more polypeptides through the thiazolidine conjugation moiety. The linker may be bound (e.g., covalently bonded) to the conjugation moiety (e.g., as described herein) at any convenient position. For example, the linker may attach the conjugation moiety to a drug. The conjugation moiety may be used to conjugate the linker (and thus the drug) to a polypeptide, such as an antibody. For example, the conjugation moiety may be used to conjugate the linker (and thus the drug) to an amino acid residue of the polypeptide (e.g., antibody), as described herein.

In some instances, as shown in formula (I) above, L^{A} is attached to W² through the thiazolidine conjugation moiety, and thus W² is indirectly bonded to the linker L^{A} through the conjugation moiety. As described above, W² is a protein (e.g., polypeptide or antibody), and thus L^{A} is attached through the conjugation moiety, to the protein (e.g., polypeptide or antibody), e.g., the linker L^{A} is indirectly bonded to the protein (e.g., polypeptide or antibody) through the conjugation moiety.

Any convenient linker may be utilized for the linker L^{A} in the subject conjugates and compounds. In certain embodiments, the linker L^{A} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the linker L^{A} may include an alkyl or substituted alkyl group. In certain embodiments, the linker L^{A} may include an alkenyl or substituted alkenyl group. In certain embodiments, the linker L^{A} may include an alkynyl or substituted alkynyl group. In certain embodiments, the linker L^{A} may include an alkoxy or substituted alkoxy group. In certain embodiments, the linker L^{A} may include an amino or substituted amino group. In certain embodiments, the linker L^{A} may include a carboxyl or carboxyl ester group. In certain embodiments, the linker L^{A} may include an acyl amino group. In certain embodiments, the linker L^{A} may include an alkylamide or substituted alkylamide group. In certain embodiments, the linker L^{A} may include an aryl or substituted aryl group. In certain embodiments, the linker L^{A} may include a heteroaryl or substituted heteroaryl group. In certain embodiments, the linker L^{A} may include a cycloalkyl or substituted cycloalkyl group. In certain embodiments, the linker L^{A} may include a heterocyclyl or substituted heterocyclyl group.

In certain embodiments, the linker L^{A} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{A} is a linker (e.g., a first linker) described by the formula:

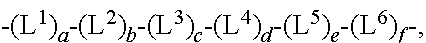

wherein L¹, L², L³, L⁴, L⁵ and L⁶ are each independently a linker subunit, and a, b, c, d, e and f are each independently 0 or 1, wherein the sum of a, b, c, d, e and f is 1 to 6.

In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the thiazolidine conjugation moiety. In certain embodiments, the linker subunit L², if present, is attached to W¹ (e.g., as shown in formula (I) above). In certain embodiments, the linker subunit L³, if present, is attached to W¹(e.g., as shown in formula (I) above). In certain embodiments, the linker subunit L⁴, if present, is attached to W¹ (e.g., as shown in formula (I) above). In certain embodiments, the linker subunit L⁵, if present, is attached to W¹ (e.g., as shown in formula (I) above). In certain embodiments, the linker subunit L⁶, if present, is attached to W¹ (e.g., as shown in formula (I) above).

Any convenient linker subunits may be utilized in the linker L^{A}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L^{A} is a linker comprising -(L¹)ₐ-(L²)_{b}-(L³)_{c}-(L⁴)_{d}-(L⁵)ₑ-(L⁶)_{f}-, where:

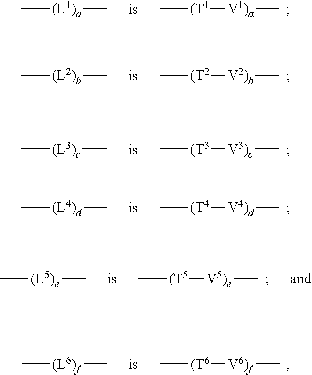

wherein T¹, T², T³, T⁴, T^{s} and T⁶, if present, are tether groups;
V¹, V², V³, V⁴, V⁵ and V⁶, if present, are covalent bonds or linking functional groups; and
a, b, c, d, e and f are each independently 0 or 1, wherein the sum of a, b, c, d, e and f is 1 to 6.

As described above, in certain embodiments, L¹ is attached to the thiazolidine conjugation moiety (e.g., as shown in formula (I) above). As such, in certain embodiments, T¹ is attached to the conjugation moiety (e.g., as shown in formula (I) above). In certain embodiments, V¹ is attached to W¹(e.g., as shown in formula (I) above). In certain embodiments, L², if present, is attached to W¹ (e.g., as shown in formula (I) above). As such, in certain embodiments, T², if present, is attached to W¹ (e.g., as shown in formula (I) above), or V², if present, is attached to W¹(e.g., as shown in formula (I) above). In certain embodiments, L³, if present, is attached to W¹ (e.g., as shown in formula (I) above). As such, in certain embodiments, T³, if present, is attached to W¹ (e.g., as shown in formula (I) above), or V³, if present, is attached to W¹(e.g., as shown in formula (I) above). In certain embodiments, L⁴, if present, is attached to W¹(e.g., as shown in formula (I) above). As such, in certain embodiments, T⁴, if present, is attached to W¹ (e.g., as shown in formula (I) above), or V⁴, if present, is attached to W¹(e.g., as shown in formula (I) above). In certain embodiments, L⁵, if present, is attached to W¹(e.g., as shown in formula (I) above). As such, in certain embodiments, T⁵, if present, is attached to W¹ (e.g., as shown in formula (I) above), or V⁵, if present, is attached to W¹(e.g., as shown in formula (I) above). In certain embodiments, L⁶, if present, is attached to W¹(e.g., as shown in formula (I) above). As such, in certain embodiments, T⁶, if present, is attached to W¹ (e.g., as shown in formula (I) above), or V⁶, if present, is attached to W¹(e.g., as shown in formula (I) above).

Regarding the tether groups, T¹, T², T³, T⁴, T⁵ and T⁶, any convenient tether groups may be utilized in the subject linkers. In some embodiments, T¹, T², T³, T⁴, T⁵ and T⁶ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), an acetal group, a hydrazine, a disulfide, and an ester, where each w is an integer from 1 to 20, each n is an integer from 1 to 30, each p is an integer from 1 to 20, and each m is an integer from 1 to 12.

In certain embodiments, the tether group (e.g., T¹, T², T³, T⁴, T⁵ and/or T⁶) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₙ group as described herein (e.g., —CONH(PEG)₃ or —NHCO(PEG)₇), or may include C₁-C₁₂ alkylene (e.g., C₃-alkylene) substituted with a —CONHCH₂CH₂SO₃H group, or may include C₁-C₁₂ alkylene (e.g., C₅-alkylene) substituted with a —NHCOCH₂SO₃H group.

In certain embodiments, the tether group (e.g., T¹, T², T³, T⁴, T⁵ and/or T⁶) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵ and T⁶) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵ and/or T⁶) includes a heteroaryl or substituted heteroaryl. In some instances, the tether group (e.g., T¹, T², T³, T⁴, T⁵ and T⁶) includes a cycloalkyl or substituted cycloalkyl. In some instances, the tether group (e.g., T¹, T², T³, T⁴, T⁵ and T⁶) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵ and/or T⁶) includes an aryl, substituted aryl, heteroaryl, substituted heteroaryl, cycloalkyl, substituted cycloalkyl, heterocyclyl, or substituted heterocyclyl, where the aryl, substituted aryl, heteroaryl, substituted heteroaryl, cycloalkyl, substituted cycloalkyl, heterocyclyl, or substituted heterocyclyl is produced from a reaction, such as a click-chemistry reaction. For example, click-chemistry reactions may be used to connect two portions of a linker together during synthesis of the linker. Examples of click-chemistry reactions that can be used to produce an aryl, substituted aryl, heteroaryl, substituted heteroaryl, cycloalkyl, substituted cycloalkyl, heterocyclyl, or substituted heterocyclyl tether group include, but are not limited to, azide-alkyne reactions, alkyne-DBCO (dibenzocyclooctyne) reactions, TCO-Tz (trans-cycloctene tetrazine) reactions, and the like.

In certain embodiments, the tether group (e.g., T¹, T², T³, T⁴, T⁵ and/or T⁶) includes an ethylene diamine (EDA) moiety, e.g., an EDA containing tether group. In certain embodiments, (EDA)_{W} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

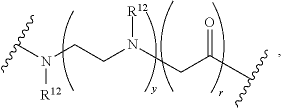

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵ and/or T⁶) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

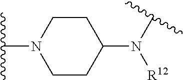

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

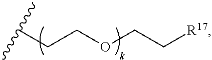

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, OR, COOH, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OR, such as OCH₃.

In certain embodiments, a tether group (e.g., T¹, T², T³, T⁴, T⁵ and/or T⁶) includes (PEG)ₙ, where (PEG)ₙ is a polyethylene glycol or a modified polyethylene glycol linking unit. In certain embodiments, (PEG)ₙ is described by the structure:

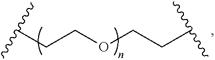

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵ and/or T⁶) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵ and/or T⁶) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, GIn or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵ and/or T⁶) includes a moiety described by the formula —(CR¹³OH)ₘ—, where m is 0 or n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, m is 1. In certain embodiments, m is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³

Regarding the linking functional groups, V¹, V², V³, V⁴, V⁵ and V⁶, any convenient linking functional groups may be utilized in the linker. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵

In certain embodiments, the tether group includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

As described above, in some embodiments, L^{A} is a linker comprising -(T¹-V¹)ₐ-(T²-V²)_{b}-(T³-V³)_{c}-(T⁴-V⁴)_{d}-(T⁵-V⁵)ₑ- (T⁶-V⁶)_{f}-, where a, b, c, d, e and f are each independently 0 or 1, where the sum of a, b, c, d, e and f is 1 to 6.

In some embodiments, in the linker L^{A}:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, -(CR¹³OH)ₘ-, 4-amino-piperidine (4AP), an acetal group, a disulfide, a hydrazine, and an ester; and
V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
wherein:
(PEG)ₙ is

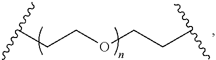

where n is an integer from 1 to 30; EDA is an ethylene diamine moiety having the following structure:

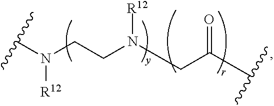

where y is an integer from 1 to 6 and r is 0 or 1;
4-amino-piperidine (4AP) is

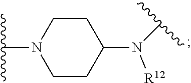

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, in the linker L^{A}: (PEG)ₙ is

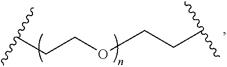

where n is an integer from 1 to 30;
EDA is an ethylene diamine moiety having the following structure:

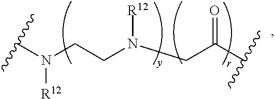

where y is an integer from 1 to 6 and r is 0 or 1;
4-amino-piperidine (4AP) is

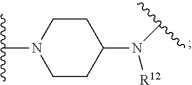

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

In certain embodiments of the conjugate of formula (I), the polypeptide (e.g., antibody) can be linked to one drug or active agent through the conjugation moiety and linker. In some instances, the polypeptide (e.g., antibody) can be linked to more than one drug or active agent through the conjugation moiety and linker. For example, the conjugation moiety can be linked to two or more drugs or active agents. Each drug or active agent can be linked via a corresponding linker to the same conjugation moiety, which in turn can be attached to the polypeptide (e.g., antibody) as described herein, thus linking the polypeptide (e.g., antibody) to two or more drugs or active agents.

For example, in certain embodiments of the conjugate of formula (I), the linker, L^{A} is a branched linker. In embodiments having a branched linker, one of T¹, T², T³, T⁴, T⁵, T⁶, V¹, V², V³, V⁴, V⁵ or V⁶ is a branched group. In some embodiments, the branched group is selected from —CONR¹⁵— and 4AP. In some embodiments, the branched group is —CONR¹⁵-. In some embodiments, the branched group is 4AP. In certain embodiments, the branched group is attached to a second linker, L^{B}. As such, the branched group of the first linker, L^{A}, can be attached to a first drug, W¹, as shown in formula (I) above, and also attached to a second drug, W^{1a}, through the second linker, L^{B} (e.g., as shown in formula (II) below).

In certain embodiments, the second linker L^{B} is a linker described by the formula:

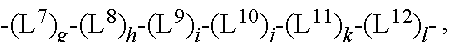

wherein L⁷, L⁸, L⁹, L¹⁰, L¹¹ and L¹² are each independently a linker subunit, and g, h, i, j, k and I are each independently 0 or 1, wherein the sum of g, h, i, j, k and I is 1 to 6.

In certain embodiments, the sum of g, h, i, j, k and I is 1. In certain embodiments, the sum of g, h, i, j, k and I is 2. In certain embodiments, the sum of g, h, i, j, k and I is 3. In certain embodiments, the sum of g, h, i, j, k and I is 4. In certain embodiments, the sum of g, h, i, j, k and I is 5. In certain embodiments, the sum of g, h, i, j, k and I is 6. In certain embodiments, g, h, i, j, k and I are each 1. In certain embodiments, g, h, i, j and k are each 1 and I is 0. In certain embodiments, g, h, i and j are each 1 and k and I are each 0. In certain embodiments, g, h, and i are each 1 and j, k and I are each 0. In certain embodiments, g and h are each 1 and i, j, k and I are each 0. In certain embodiments, g is 1 and h, i, j, k and I are each 0.

In certain embodiments, the linker subunit L⁷ is attached to the branched group. In certain embodiments, the linker subunit L⁸, if present, is attached to the second drug, W^{1a}. In certain embodiments, the linker subunit L⁹, if present, is attached to the second drug, W^{1a}. In certain embodiments, the linker subunit L¹⁰, if present, is attached to the second drug, W^{1a}. In certain embodiments, the linker subunit L¹¹, if present, is attached to the second drug, W^{1a}. In certain embodiments, the linker subunit L¹², if present, is attached to the second drug, W^{1a}.

Any convenient linker subunits may be utilized in the second linker L^{B}. For example, any of the linker subunits described above in relation to L¹, L², L³, L⁴, L⁵ and L⁶ may be used for the linker subunits L⁷, L⁸, L⁹, L¹⁰, L¹¹ and L¹².

In certain embodiments, the second linker L^{B} is a linker comprising:
(L⁷)_{g}-(L⁸)ₕ(L⁹)ᵢ-L¹⁰)ⱼ(L¹¹)ₖ-L¹²)ₗ-, where:

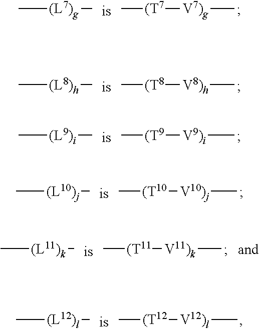

wherein T⁷, T⁸, T⁹, T¹⁰, T¹¹ and T¹², if present, are tether groups;
V⁷, V⁸, V⁹, V¹⁰, V¹¹ and V¹², if present, are covalent bonds or linking functional groups; and
g, h, i, j, k and I are each independently 0 or 1, wherein the sum of g, h, i, j, k and I is 1 to 6.

Accordingly, in certain embodiments, the second linker L^{B} comprises:

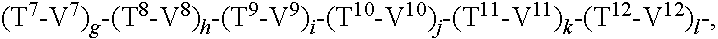

wherein
g, h, i, j, k and 1 are each independently 0 or 1;
T⁷, T⁸, T⁹, T¹⁰, T¹¹ and T¹² are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V⁷, V⁸, V⁹, V¹⁰, V¹¹ and V¹² are each independently selected from the group consisting of a covalent bond, —CO—, —NR¹⁵, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

Any convenient tether groups may be utilized for T⁷, T⁸, T⁹, T¹⁰, T¹¹ and T¹². For example, any of the tether groups described above in relation to T¹, T², T³, T⁴, T⁵ and T⁶ may be used for the tether groups T⁷, T⁸, T⁹, T¹⁰, T¹¹ and T¹².

Any convenient linking functional groups may be utilized for V⁷, V⁸, V⁹, V¹⁰, V¹¹ and V¹². For example, any of the linking functional groups described above in relation to V¹, V², V³, V⁴, V⁵ and V⁶ may be used for the linking functional groups V⁷, V⁸, V⁹, V¹⁰, V¹¹ and V¹².

In certain embodiments, each R¹³ is independently selected from hydrogen, alkyl, substituted alkyl, aryl, and substituted aryl. In these embodiments, alkyl, substituted alkyl, aryl, and substituted aryl are as described above for R¹³.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵. In these embodiments, various possible substituents are as described above for R¹⁵

As described above, in certain embodiments, the branched group is attached to a second linker, L^{B}. In addition, the second linker, L^{B}, can be attached to the second drug, W^{1a}, as shown in formula (II):

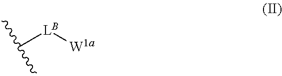

wherein:
L^{B} is a second linker; and
W^{1a} is a drug.

Accordingly, L^{B} may be attached to a compound of formula (I) through a branched group. For example, L^{B} may be attached to a compound of formula (I) at the bond indicated by the wavy line in formula (II).

The substituents related to conjugates of formula (II) are described in more detail below.

In certain embodiments, W^{1a} is a drug (or active agent). In certain embodiments of the second linker L^{B}, the left-hand side of the linker structure is attached to the branched group, and the right-hand side of the linker structure is attached to the second drug, W^{1a}, as shown in formula (II). Further description of drugs (or active agents) that find use in the subject conjugates is found in the disclosure herein.

Combinations of the same or different payloads (e.g., drugs or active agents) may be conjugated to the polypeptide (e.g., antibody) through the branched linker. In certain embodiments, the two payloads (e.g., drugs or active agents) attached to the branched linker are the same payload (e.g., drug or active agent). For example, a first branch of a branched linker may be attached to a payload (e.g., drug or active agent) and a second branch of the branched linker may be attached to the same payload (e.g., drug or active agent) as the first branch. In other embodiments, the two payloads (e.g., drugs or active agents) attached to the branched linker are different payloads (e.g., drugs or active agents). For example, a first branch of a branched linker may be attached to a first payload (e.g., drug or active agent) and a second branch of the branched linker may be attached to a second payload (e.g., drug or active agent) different from the first payload (e.g., drug or active agent) attached to the first branch.

For example, in certain embodiments, the branched linker is attached to a first drug, W¹, and a second drug, W^{1a}. In some cases, W¹ and W^{1a} are the same drug. In other cases, W¹ and W^{1a} are different drugs.

Various embodiments of the linkers that may couple the conjugation moiety to the drugs or active agents are described in detail herein. For example, in some instances, the linker is a cleavable linker, such as a cleavable linker as described herein.

In certain embodiments, the conjugate is an antibody-drug conjugate where the antibody and the drug are linked together by a linker (e.g., L^{A} and/or L^{B}), as described above. In some instances, the linker is a cleavable linker. A cleavable linker is a linker that includes one or more cleavable moieties, where the cleavable moiety includes one or more bonds that can dissociate under certain conditions, thus separating the cleavable linker into two or more separatable portions. For example, the cleavable moiety may include one or more covalent bonds, which under certain conditions, can dissociate or break apart to separate the cleavable linker into two or more portions. As such a cleavable linker can be included in an antibody-drug conjugate, such that under appropriate conditions, the cleavable linker is cleaved to separate or release the drug from the antibody at a desired target site of action for the drug.

In some instances, the cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of the cleavable linker can be achieved by initially cleaving one of the two cleavable moieties and then cleaving the other of the two cleavable moieties. In certain embodiments, the cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety by exposing the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (i.e., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas (e.g., systemically) or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as the protease enzyme cathepsin B, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (i.e., the site of the cancer (and overexpressed enzyme)). Thus, in some embodiments, the enzymatically cleavable moiety is a cleavable moiety (e.g., a peptide) that can be cleaved by an enzyme that is overexpressed in cancer cells. For instance, the enzyme can be the protease enzyme cathepsin B. As such, in some instances, the enzymatically cleavable moiety is a cleavable moiety (e.g., a peptide) that can be cleaved by a protease enzyme, such as cathepsin B.

In certain embodiments, the enzymatically cleavable moiety is a peptide. The peptide can be any peptide suitable for use in the cleavable linker and that can be cleaved through the enzymatic action of an enzyme. Non-limiting examples of peptides that can be used as an enzymatically cleavable moiety include, for example, Val-Ala, Phe-Lys, and the like. For example, the first cleavable moiety described above (i.e., the cleavable moiety protected from premature cleavage by the second cleavable moiety) can include a peptide. The presence of uncleaved second cleavable moiety can protect the first cleavable moiety (peptide) from cleavage by a protease enzyme (e.g., cathepsin B), and thus substantially reduce or prevent premature release of the drug from the antibody until the antibody-drug conjugate is at or near the desired target site of action for the drug. In some instances, one of the amino acid residues of the peptide that comprises the first cleavable moiety is linked to or includes a substituent, where the substituent comprises the second cleavable moiety. In some instances, the second cleavable moiety includes a glycoside.

For example, in the context of the formulae described herein, the cleavable moiety may be a peptide, such as a peptide described by the moiety:

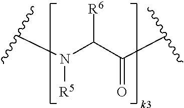

wherein
each R⁵ is independently selected from hydrogen, alkyl, substituted alkyl, alkenyl, substituted alkenyl, alkynyl, and substituted alkynyl;
each R⁶ is independently selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl; and
k3 is an integer from 1 to 10.

In certain embodiments, each R⁵ is independently selected from hydrogen, alkyl, substituted alkyl, alkenyl, substituted alkenyl, alkynyl, and substituted alkynyl. In certain embodiments, R⁵ is hydrogen. In certain embodiments, R⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂-3 substituted alkenyl. In certain embodiments, R⁵ is alkynyl or substituted alkynyl.

In certain embodiments, each R⁶ is independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R⁶ is hydrogen. In certain embodiments, R⁶ is alkyl or substituted alkyl, such as C₁₋₆ alkyl or C₁₋₆ substituted alkyl, or C₁₋₄ alkyl or C₁_4 substituted alkyl, or C₁₋₃ alkyl or C₁₋₃ substituted alkyl. In certain embodiments, R⁶ is methyl. In certain embodiments, R⁶ is isopropyl. In certain embodiments, R⁶ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁶ is alkynyl or substituted alkynyl. In certain embodiments, R⁶ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R⁶ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁶ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁶ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R⁵ is H and R⁶ is alkyl or substituted alkyl. For example, in some instances, R⁵ is H and R⁶ is methyl; or R⁵ is H and R⁶ is isopropyl.

In certain embodiments, k3 is an integer from 1 to 10. In certain embodiments, k3 is 1. In certain embodiments, k3 is 2. In certain embodiments, k3 is 3. In certain embodiments, k3 is 4. In certain embodiments, k3 is 5. In certain embodiments, k3 is 6. In certain embodiments, k3 is 7. In certain embodiments, k3 is 8. In certain embodiments, k3 is 9. In certain embodiments, k3 is 10.

In certain embodiments, k3 is 2, and each R⁵ is H and each R⁶ is alkyl or substituted alkyl. For example, in some instances, k3 is 2, and each R⁵ is H and one R⁶ is methyl and the other R⁶ is isopropyl.

The above peptide cleavable moiety may be included in one or more of the linkers (e.g., L^{A} and/or L^{B}) described herein.

In some embodiments, the enzymatically cleavable moiety is sugar moiety, such as a glycoside (or glyosyl). In some cases, the glycoside can facilitate an increase in the hydrophilicity of the cleavable linker as compared to a cleavable linker that does not include the glycoside. The glycoside can be any glycoside or glycoside derivative suitable for use in the cleavable linker and that can be cleaved through the enzymatic action of an enzyme. For example, the second cleavable moiety (i.e., the cleavable moiety that protects the first cleavable moiety from premature cleavage) can be a glycoside or glycoside derivative. For instance, in some embodiments, the first cleavable moiety includes a peptide and the second cleavable moiety includes a glycoside or glycoside derivative. In certain embodiments, the second cleavable moiety is a glycoside or glycoside derivative selected from a glucuronide, a galactoside, a glucoside, a mannoside, a fucoside, O-GIcNAc, and O-GaINAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GIcNAc. In some instances, the second cleavable moiety is O-GaINAc.

The glycoside or glycoside derivative can be attached (covalently bonded) to the cleavable linker through a glycosidic bond. The glycosidic bond can link the glycoside or glycoside derivative to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside or glycoside derivative can be cleaved from the cleavable linker it is attached to by an enzyme (e.g., through enzymatically-mediated hydrolysis of the glycosidic bond). A glycoside or glycoside derivative can be removed or cleaved from the cleavable linker by any convenient enzyme that is able to carry out the cleavage (hydrolysis) of the glycosidic bond that attaches the glycoside or glycoside derivative to the cleavable linker. An example of an enzyme that can be used to mediate the cleavage (hydrolysis) of the glycosidic bond that attaches the glycoside or glycoside derivative to the cleavable linker is a glucuronidase, a glycosidase, such as a galactosidase, a glucosidase, a mannosidase, a fucosidase, and the like. Other suitable enzymes may also be used to mediate the cleavage (hydrolysis) of the glycosidic bond that attaches the glycoside or glycoside derivative to the cleavable linker. In some cases, the enzyme used to mediate the cleavage (hydrolysis) of the glycosidic bond that attaches the glycoside or glycoside derivative to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

Embodiments that include a linker with the two cleavable moieties as described above can also be referred to as having a “tandem-cleavage” linker.

In certain embodiments, the glycoside or glycoside derivative is selected from a glucuronide, a galactoside, a glucoside, a mannoside, a fucoside, O-GIcNAc, and O-GaINAc. For example, in some embodiments, the glycoside or glycoside derivative can be selected from the following structures:

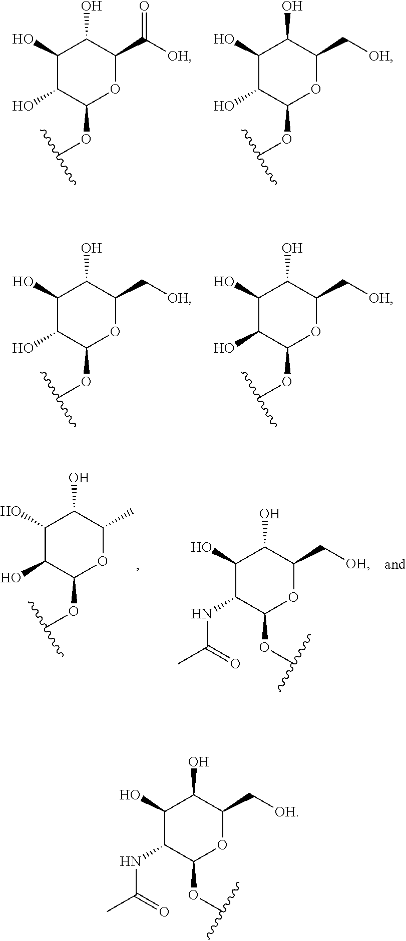

In certain embodiments, the conjugate of formula (I) has a structure selected from the following:

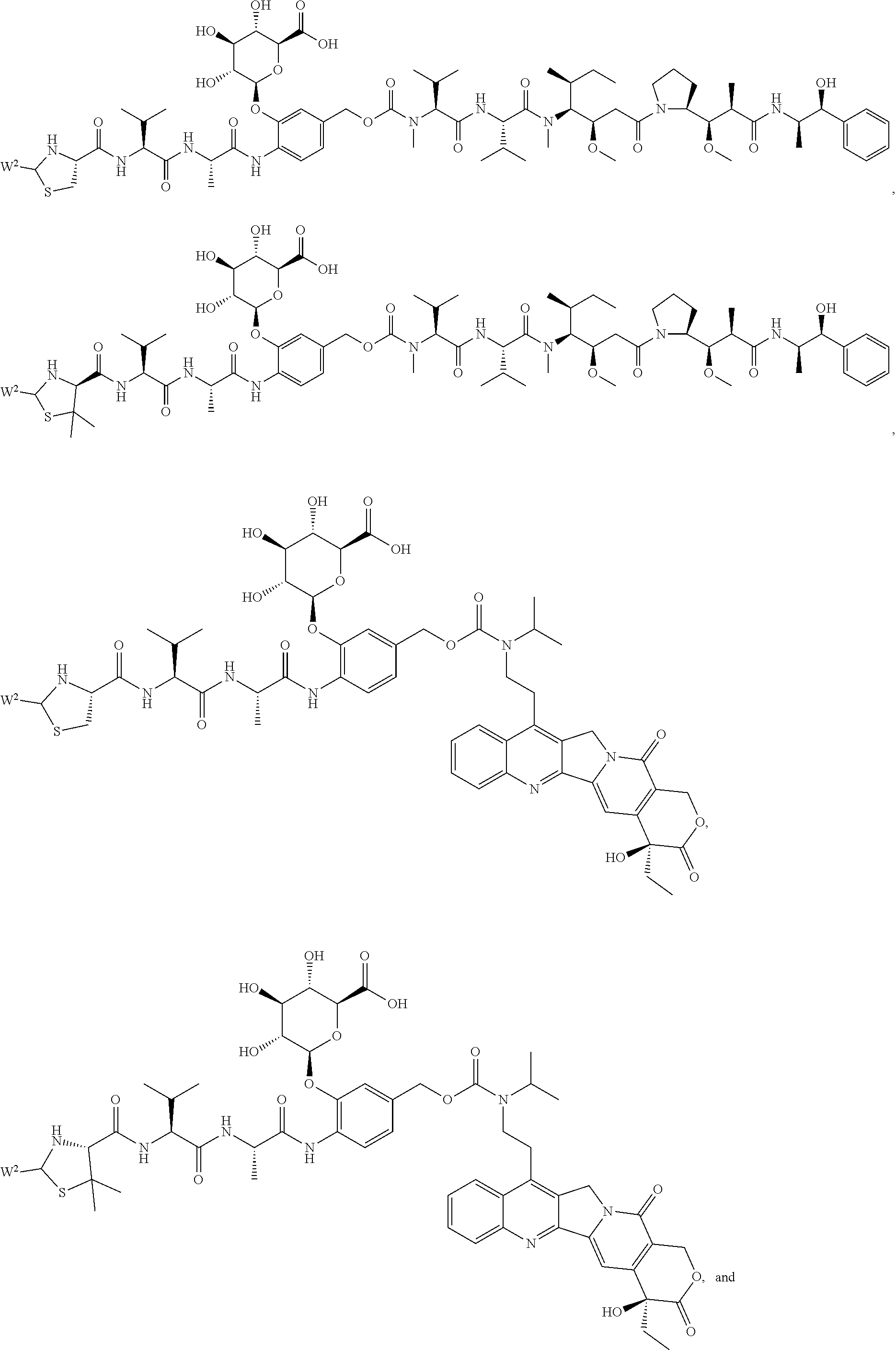

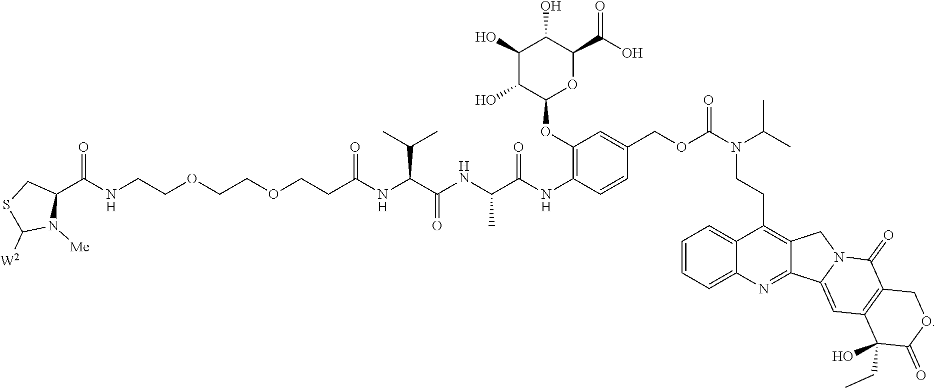

Any of the chemical entities, linkers, conjugation moieties, or cleavable moieties set forth in the description above may be adapted for use in the subject compounds and conjugates.

Additional disclosure related to cleavable linkers is found in U.S. Application Publication No. 2022/0226490, and U.S. Application Publication No. 2022/0249686, the disclosures of each of which are incorporated herein by reference.

### Compounds Useful for Producing Conjugates

The present disclosure provides compounds useful for producing the conjugates described herein. In certain embodiments, the compound may include a conjugation moiety useful for conjugation of a polypeptide (e.g., an antibody) and a drug or active agent. For example, the compound may be bound to the polypeptide (antibody) and also bound to the drug or active agent, thus indirectly attaching the polypeptide (antibody) and the drug together.

In certain embodiments, the compound is a compound of formula (III):

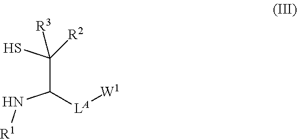

wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker; and
W¹ is a drug.

Regarding compounds of formula (III), the substituents R¹, R², R³, L^{A}, and W¹ are as described above in relation to the conjugates of formula (I). Similarly, regarding the first linker L^{A} and the second linker L^{B}, the T¹, T², T³, T⁴, T⁵, T⁶, V¹, V², V³, V⁴, V⁵ and V⁶, and T⁷, T⁸, T⁹, T¹⁰, T¹¹, T¹², V⁷, V⁸, V⁹, V¹⁰, V¹¹ and V¹² substituents are as described above in relation to the conjugates of formula (I).

As described herein, in certain embodiments, the branched group is attached to a second linker, L^{B}. In addition, the second linker, L^{B}, can be attached to the second drug, W^{1a}, as shown in formula (IV):

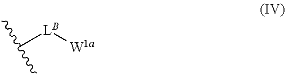

wherein:
L^{B} is a second linker; and
W^{1a} is a drug.

Accordingly, L^{B} may be attached to a compound of formula (III). For example, L^{B} may be attached to a compound of formula (III) at the bond indicated by the wavy line in formula (IV).

In embodiments of formula (IV), L^{B} and W^{1a} are as described herein (e.g., as in the description related to formula (II) herein).

Compounds of formula (III) can be used in conjugation reactions described herein, where a drug or active agent attached to a conjugation moiety is conjugated to a polypeptide (e.g., antibody) to form an antibody-drug conjugate.

In certain embodiments, the compound of formula (III) has the following structure:

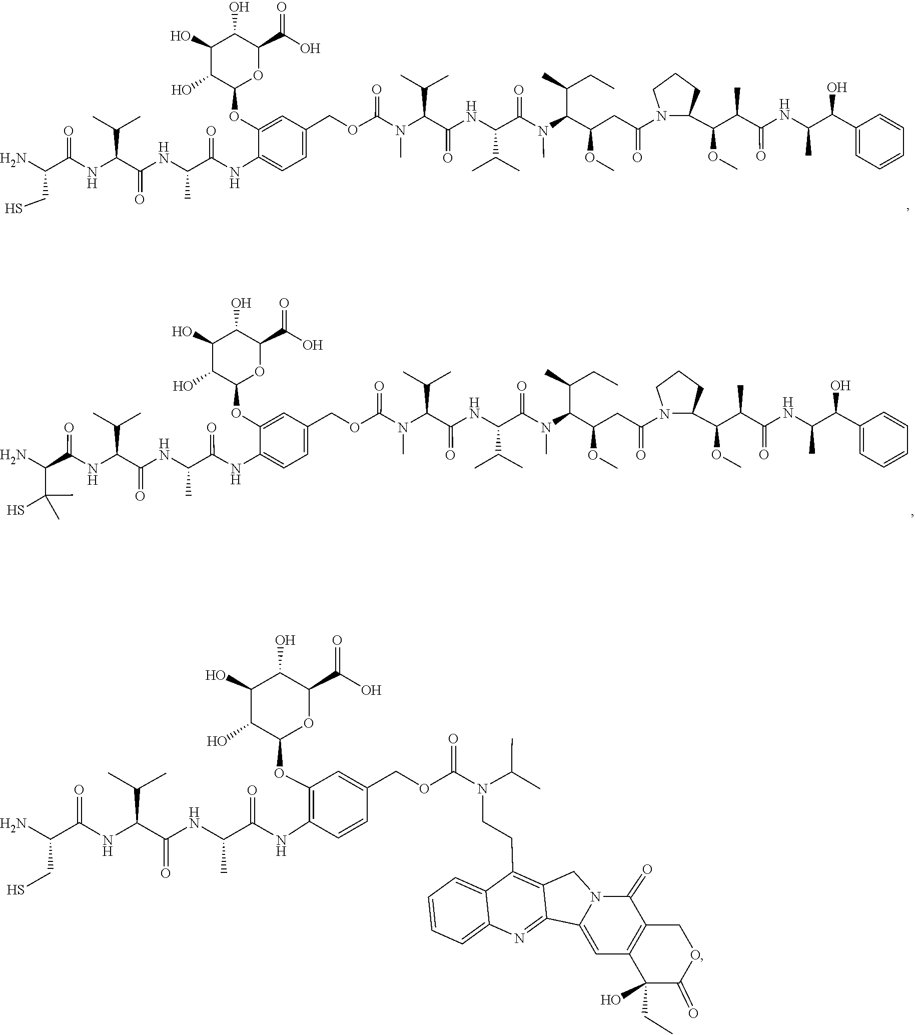

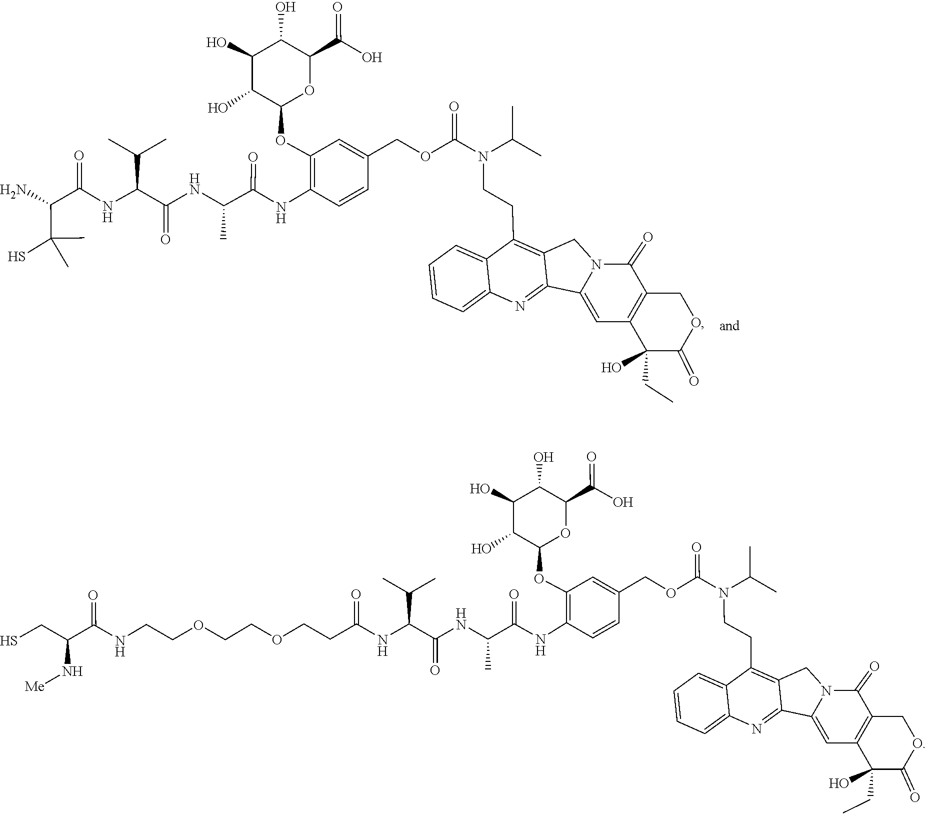

Any of the chemical entities, linkers, conjugation moieties and cleavable moieties set forth in the description above may be adapted for use in the subject compounds and conjugates.

### Polypeptides and Antibodies

As noted above, a subject conjugate can comprise as substituent W² a peptide (e.g., a polypeptide or an antibody). As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or lie or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

In certain embodiments, the amino acid sequence of the polypeptide (antibody) has been modified to include one or more 2-formylglycine (fGly) residues. In certain embodiments, the amino acid sequence of the polypeptide (antibody) is modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8, 7 or 6 amino acid residues in length.

In certain embodiments, polypeptides of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the polypeptide. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the polypeptide. Where an amino acid sequence native to the polypeptide (e.g., antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target antibody is minimized, so as to minimize the number of amino acid residues that are inserted, deleted, substituted (replaced), or added (e.g., to the N- or C-terminus). Minimizing the extent of amino acid sequence modification of the target antibody may minimize the impact such modifications may have upon antibody function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH₂ domain of an Ig heavy chain. An aldehyde tag can be present within a CH3 domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

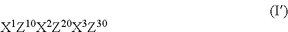

where
Z¹⁰ is cysteine or serine (which can also be represented by (C/S));
Z²⁰ is either a proline or alanine residue (which can also be represented by (P/A));
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I;
X¹ is present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M, S or V, with the proviso that when the sulfatase motif is at the N-terminus of the target polypeptide, X¹ is present; and
X² and X³ independently can be any amino acid, though usually an aliphatic amino acid, a polar, uncharged amino acid, or a sulfur containing amino acid (i.e., other than an aromatic amino acid or a charged amino acid), e.g., S, T, A, V, G or C, e.g., S, T, A, V or G.

The amino acid sequence of an antibody heavy and/or light chain can be modified to provide a sequence of at least 5 amino acids of the formula X¹Z¹⁰X²Z²⁰X³Z³⁰, where
Z¹⁰ is cysteine or serine;
Z²⁰ is a proline or alanine residue;
Z³⁰ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid.

The sulfatase motif is generally selected so as to be capable of conversion by a selected FGE, e.g., an FGE present in a host cell in which the aldehyde tagged polypeptide is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

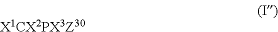

where
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G, or C, e.g., S, T, A, V or G; and
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO:3), MCTPSR (SEQ ID NO:25), VCTPSR (SEQ ID NO:4), LCSPSR (SEQ ID NO:5), LCAPSR (SEQ ID NO:6), LCVPSR (SEQ ID NO:7), LCGPSR (SEQ ID NO:8), ICTPAR (SEQ ID NO:9), LCTPSK (SEQ ID NO:10, MCTPSK (SEQ ID NO:11), VCTPSK (SEQ ID NO:12), LCSPSK (SEQ ID NO:13), LCAPSK (SEQ ID NO:14), LCVPSK (SEQ ID NO:15), LCGPSK (SEQ ID NO:16), LCTPSA (SEQ ID NO:17), ICTPAA (SEQ ID NO:18), MCTPSA (SEQ ID NO:19), VCTPSA (SEQ ID NO:20), LCSPSA (SEQ ID NO:21), LCAPSA (SEQ ID NO:22), LCVPSA (SEQ ID NO:23), and LCGPSA (SEQ ID NO:24).

fGly-Containing Sequences

Upon action of FGE on the antibody heavy and/or light chain, the serine or the cysteine in the sulfatase motif is modified to fGly. Thus, the fGly-containing sulfatase motif can be of the formula:

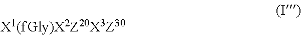

where
fGly is the formylglycine residue;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

As described above, to produce the conjugate, the polypeptide containing the fGly residue may be conjugated to a drug or active agent by reaction of the fGly with a reactive moiety (e.g., a conjugation moiety, as described herein) of a linker attached to the drug or active agent to produce an fGly′-containing sulfatase motif. As used herein, the term fGly′ refers to the amino acid residue of the sulfatase motif that is coupled to the drug or active agent through a linker, as described herein. Thus, the fGly′-containing sulfatase motif can be of the formula:

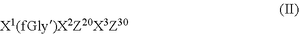

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

### Site of Modification

As noted above, in certain embodiments, the amino acid sequence of the polypeptide (antibody) is modified to include a sulfatase motif that contains a serine or cysteine residue that is capable of being converted (oxidized) to an fGly residue by action of an FGE either in vivo (e.g., at the time of translation of an aldehyde tag-containing protein in a cell) or in vitro (e.g., by contacting an aldehyde tag-containing protein with an FGE in a cell-free system). The antibody used to generate a conjugate of the present disclosure include at least an Ig constant region, e.g., an Ig heavy chain constant region (e.g., at least a CH1 domain; at least a CH1 and a CH₂ domain; a CH1, a CH₂, and a CH3 domain; or a CH1, a CH₂, a CH3, and a CH4 domain), or an Ig light chain constant region. Such Ig polypeptides are referred to herein as “target Ig polypeptides” or “target antibodies”.

The site in an antibody into which a sulfatase motif is introduced can be any convenient site. As noted above, in some instances, the extent of modification of the native amino acid sequence of the target polypeptide is minimized, so as to minimize the number of amino acid residues that are inserted, deleted, substituted (replaced), and/or added (e.g., to the N- or C-terminus). Minimizing the extent of amino acid sequence modification of the target antibody may minimize the impact such modifications may have upon antibody function and/or structure.

An antibody heavy chain constant region can include Ig constant regions of any heavy chain isotype, non-naturally occurring Ig heavy chain constant regions (including consensus Ig heavy chain constant regions). An Ig constant region amino acid sequence can be modified to include an aldehyde tag, where the aldehyde tag is present in or adjacent a solvent-accessible loop region of the Ig constant region. An Ig constant region amino acid sequence can be modified by insertion and/or substitution of 1, 2, 3, 4, 5, 6, 7, 8, 9, 10, 11, 12, 13, 14, 15, or 16 amino acids, or more than 16 amino acids, to provide an amino acid sequence of a sulfatase motif as described above.

In some cases, an aldehyde-tagged antibody comprises an aldehyde-tagged Ig heavy chain constant region (e.g., at least a CH1 domain; at least a CH1 and a CH₂ domain; a CH1, a CH₂, and a CH3 domain; or a CH1, a CH₂, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc., modified to include at least one sulfatase motif that can be modified by an FGE to generate an fGly-modified Ig polypeptide. Allotypic variants of Ig heavy chains are known in the art. See, e.g., Jefferis and Lefranc (2009) MAbs 1:4.

In some cases, an aldehyde-tagged antibody comprises an aldehyde-tagged Ig light chain constant region. The aldehyde-tagged Ig light chain constant region can include constant region sequences of a kappa light chain, a lambda light chain, e.g., human kappa or lambda light chain constant regions, a hybrid light chain constant region, a synthetic light chain constant region, or a consensus light chain constant region sequence, etc., that includes at least one sulfatase motif that can be modified by an FGE to generate an fGly-modified antibody. Exemplary constant regions include human gamma 1 and gamma 3 regions. With the exception of the sulfatase motif, a modified constant region may have a wild-type amino acid sequence, or it may have an amino acid sequence that is at least 70% identical (e.g., at least 80%, at least 90% or at least 95% identical) to a wild type amino acid sequence.

In some embodiments the sulfatase motif is at a position other than, or in addition to, the C-terminus of the Ig polypeptide heavy chain. As noted above, an isolated aldehyde-tagged antibody can comprise a heavy chain constant region amino acid sequence modified to include a sulfatase motif as described above, where the sulfatase motif is in or adjacent a surface-accessible loop region of the antibody heavy chain constant region.

A sulfatase motif can be provided within or adjacent one or more of these amino acid sequences of such modification sites of an Ig heavy chain. For example, an Ig heavy chain polypeptide amino acid sequence can be modified (e.g., where the modification includes one or more amino acid residue insertions, deletions, and/or substitutions) at one or more of these amino acid sequences to provide a sulfatase motif adjacent and N-terminal and/or adjacent and C-terminal to these modification sites. Alternatively or in addition, an Ig heavy chain polypeptide amino acid sequence can be modified (e.g., where the modification includes one or more amino acid residue insertions, deletions, and/or substitutions) at one or more of these amino acid sequences to provide a sulfatase motif between any two residues of the Ig heavy chain modifications sites. In some embodiments, an Ig heavy chain polypeptide amino acid sequence may be modified to include two motifs, which may be adjacent to one another, or which may be separated by one, two, three, four or more (e.g., from about 1 to about 25, from about 25 to about 50, or from about 50 to about 100, or more, amino acids. Alternatively or in addition, where a native amino acid sequence provides for one or more amino acid residues of a sulfatase motif sequence, selected amino acid residues of the modification sites of an Ig heavy chain polypeptide amino acid sequence can be modified (e.g., where the modification includes one or more amino acid residue insertions, deletions, and/or substitutions) so as to provide a sulfatase motif at the modification site.

An antibody used in an antibody-drug conjugate of the present disclosure can have any of a variety of antigen-binding specificities, including but not limited to, e.g., an antigen present on a cancer cell; an antigen present on an autoimmune cell; an antigen present on a pathogenic microorganism; an antigen present on a virus-infected cell (e.g., a human immunodeficiency virus-infected cell); an antigen present on a diseased cell; and the like. For example, an antibody conjugate can bind an antigen, where the antigen is present on the surface of the cell. An antibody conjugate of the present disclosure can bind antigen with a suitable binding affinity, e.g., from 5×10⁻⁶ M to 10⁻⁷ M, from 10⁻⁷ M to 5×10⁻⁷ M, from 5×10⁻⁷ M to 10⁻⁸ M, from 10⁻⁸ M to 5×10⁻⁸ M, from 5×10⁻⁸ M to 10⁻⁹ M, or a binding affinity greater than 10⁻⁹ M.

As non-limiting examples, a subject antibody conjugate can bind an antigen present on a cancer cell (e.g., a tumor-specific antigen; an antigen that is over-expressed on a cancer cell; etc.), and the conjugated moiety can be a drug, such as a cytotoxic compound (e.g., a cytotoxic small molecule, a cytotoxic synthetic peptide, etc.). For example, a subject antibody conjugate can be specific for an antigen on a cancer cell, where the conjugated moiety is a drug, such as a cytotoxic compound (e.g., a cytotoxic small molecule, a cytotoxic synthetic peptide, etc.).

As further non-limiting examples, a subject antibody conjugate can bind an antigen present on a cell infected with a virus (e.g., where the antigen is encoded by the virus; where the antigen is expressed on a cell type that is infected by a virus; etc.), and the conjugated moiety can be a drug, such as a viral fusion inhibitor. For example, a subject antibody conjugate can bind an antigen present on a cell infected with a virus, and the conjugated moiety can be a drug, such as a viral fusion inhibitor.

### Drugs for Conjugation to a Polypeptide

As noted above, a conjugate or a compound of the present disclosure can include as substituents W¹ and W^{1a} a drug or active agent. Any of a number of drugs are suitable for use, or can be modified to be rendered suitable for use, as a reactive partner to conjugate to an antibody. Examples of drugs include small molecule drugs and peptide drugs.

“Small molecule drug” as used herein refers to a compound, e.g., an organic compound, which exhibits a pharmaceutical activity of interest and which is generally of a molecular weight of 800 Da or less, or 2000 Da or less, but can encompass molecules of up to 5 kDa and can be as large as 10 kDa. A small inorganic molecule refers to a molecule containing no carbon atoms, while a small organic molecule refers to a compound containing at least one carbon atom.

For example, the drug or active agent can be a topoisomerase inhibitor (e.g., a topoisomerase I inhibitor), such as a camptothecine, or an analog or derivative thereof, or a pharmaceutically active camptothecine moiety and/or a portion thereof. A topoisomerase inhibitor (e.g., camptothecine, or analog or derivative thereof) conjugated to the polypeptide can be any of a variety of topoisomerase inhibitors, for example camptothecine or camptothecine moieties such as, but not limited to, camptothecine and analogs and derivatives thereof as described herein. Examples of drugs that find use in the conjugates and compounds described herein include, but are not limited to, a topoisomerase inhibitor, for example camptothecine or a camptothecine derivative, such as SN-38, Belotecan, Exatecan, 9-aminocamptothecin (9-AC), topotecan, des-Me-topotecan, derivatives thereof, and the like. Additional examples of topoisomerase inhibitors that find use in the present disclosure are described in PCT/US2022/012325, the disclosure of which is incorporated herein by reference.

In other embodiments, the drug or active agent can be a maytansine. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the polypeptide can be any of a variety of maytansinoid moieties such as, but not limited to, maytansine and analogs and derivatives thereof as described herein (e.g., deacylmaytansine).

In other instances, the drug or active agent can be an auristatin, or an analog or derivative thereof, or a pharmaceutically active auristatin moiety and/or a portion thereof. An auristatin conjugated to the polypeptide can be any of a variety of auristatin moieties such as, but not limited to, an auristatin and analogs and derivatives thereof as described herein. Examples of drugs that find use in the conjugates and compounds described herein include, but are not limited to an auristatin or an auristatin derivative, such as monomethyl auristatin D (MMAD), monomethyl auristatin E (MMAE), monomethyl auristatin F (MMAF), derivatives thereof, and the like.

In other cases, the drug or active agent can be a duocarmycin, or an analog or derivative thereof, or a pharmaceutically active duocarmycin moiety and/or a portion thereof. A duocarmycin conjugated to the polypeptide can be any of a variety of duocarmycin moieties such as, but not limited to, a duocarmycin and analogs and derivatives thereof as described herein. Examples of drugs that find use in the conjugates and compounds described herein include, but are not limited to a duocarmycin or a duocarmycin derivative, such as duocarmycin A, duocarmycin B1, duocarmycin B2, duocarmycin C1, duocarmycin C2, duocarmycin D, duocarmycin SA, and CC-1065, derivatives thereof, and the like. In some embodiments, the duocarmycin is a duocarmycin analog, such as, but not limited to, adozelesin, bizelesin, or carzelesin.

In certain embodiments, the drug is selected from a cytotoxin, a kinase inhibitor, a selective estrogen receptor modulator, an immunostimulatory agent, a toll-like receptor (TLR) agonist, an oligonucleotide, an aptamer, a cytokine, a steroid, and a peptide.

For example, a cytotoxin can include any compound that leads to cell death (e.g., necrosis or apoptosis) or a decrease in cell viability.

Kinase inhibitors can include, but are not limited to, Adavosertib, Afatinib, Axitinib, Bosutinib, Cetuximab, Cobimetinib, Crizotinib, Cabozantinib, Dacomitinib, Dasatinib, Entrectinib, Erdafitinib, Erlotinib, Fostamatinib, Gefitinib, Ibrutinib, Imatinib, Lapatinib, Lenvatinib, Mubritinib, Nilotinib, Pazopanib, Pegaptanib, Ruxolitinib, Sorafenib, Sunitinib, Tucatinib, Vandetanib, Vemurafenib, and the like.

For example, selective estrogen receptor modulators include, but are not limited to, Endoxifen, Tamoxifen, Afimoxifene, Toremifene, and the like.

Immunostimulatory agents can include, but are not limited to, vaccines (e.g., bacterial or viral vaccines), colony stimulating factors, interferons, interleukins, and the like. TLR agonists include, but are not limited to, imiquimod, resiquimod, and the like.

Oligonucleotide dugs include, but are not limited to, fomivirsen, pegaptanib, mipomersen, eteplirsen, defibrotide, nusinersen, golodirsen, viltolarsen, volanesorsen, inotersen, tofersen, tominersen, and the like.

Aptamer drugs include, but are not limited to, pegaptanib, AS1411, REG1, ARC1779, NU172, ARC1905, E10030, NOX-A12, NOX-E36, and the like.

Cytokines include, but are not limited to, Albinterferon Alfa-2B, Aldesleukin, ALT-801, Anakinra, Ancestim, Avotermin, Balugrastim, Bempegaldesleukin, Binetrakin, Cintredekin Besudotox, CTCE-0214, Darbepoetin alfa, Denileukin diftitox, Dulanermin, Edodekin alfa, Emfilermin, Epoetin delta, Erythropoietin, Human interleukin-2, Interferon alfa, Interferon alfa-2c, Interferon alfa-n1, Interferon alfa-n3, Interferon alfacon-1, Interferon beta-1a, Interferon beta-1b, Interferon gamma-1b, Interferon Kappa, Interleukin-1 alpha, Interleukin-10, Interleukin-7, Lenograstim, Leridistim, Lipegfilgrastim, Lorukafusp alfa, Maxy-G34, Methoxy polyethylene glycol-epoetin beta, Molgramostim, Muplestim, Nagrestipen, Oprelvekin, Pegfilgrastim, Pegilodecakin, Peginterferon alfa-2a, Peginterferon alfa-2b, Peginterferon beta-1a, Peginterferon lambda-1a, Recombinant CD40-ligand, Regramostim, Romiplostim, Sargramostim, Thrombopoietin, Tucotuzumab celmoleukin, Viral Macrophage-Inflammatory Protein, and the like.

Steroid drugs include, but are not limited to, prednisolone, betamethasone, dexamethasone, hydrocortisone, methylprednisolone, deflazacort, and the like.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

Additional examples of drugs that find use in the conjugates and compounds described herein include, but are not limited to Tubulysin M, Calicheamicin, a STAT3 inhibitor, alpha-Amanitin, an aurora kinase inhibitor, belotecan, and an anthracycline.

Other examples of drugs include small molecule drugs, such as a cancer chemotherapeutic agent. For example, where the polypeptide is an antibody (or fragment thereof) that has specificity for a tumor cell, the antibody can be produced as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca)alkaloids, and steroid hormones. Peptidic compounds can also be used.

Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOLO, TAXOTERED (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere0 docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

Examples of drugs include small molecule drugs, such as a cancer chemotherapeutic agent. For example, where the polypeptide is an antibody (or fragment thereof) that has specificity for a tumor cell, the antibody can be produced as described herein to include a modified amino acid, which can be subsequently conjugated to a cancer chemotherapeutic agent, such as a microtubule affecting agent. In certain embodiments, the drug is a microtubule affecting agent that has antiproliferative activity, such as a maytansinoid.

Embodiments of the present disclosure include conjugates where an antibody is conjugated to one or more drug moieties, such as two or more drug moieties, such as 3 drug moieties, 4 drug moieties, 5 drug moieties, 6 drug moieties, 7 drug moieties, 8 drug moieties, 9 drug moieties, 10 drug moieties, 11 drug moieties, 12 drug moieties, 13 drug moieties, 14 drug moieties, 15 drug moieties, 16 drug moieties, 17 drug moieties, 18 drug moieties, 19 drug moieties, or 20 or more drug moieties. The drug moieties may be conjugated to the antibody at one or more sites in the antibody, as described herein. In certain embodiments, the conjugates have an average drug-to-antibody ratio (DAR) (molar ratio) in the range of from 0.1 to 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 10, such as 1, 2, 3, 4, 5, 6, 7, 8, 9, or 10. In certain embodiments, the conjugates have an average DAR from 10 to 20, such as 10, 11, 12, 13, 14, 15, 16, 17, 18, 19, or 20. In certain embodiments, the conjugates have an average DAR of 1 to 10. In certain embodiments, the conjugates have an average DAR of 1 to 5 (e.g., 4). In certain embodiments, the conjugates have an average DAR of 5 to 10 (e.g., 8). In certain embodiments, the conjugates have an average DAR of 8 to 12 (e.g., 10). In certain embodiments, the conjugates have an average DAR of 10 to 15 (e.g., 12). In certain embodiments, the conjugates have an average DAR of 15 to 20 (e.g., 16). By average is meant the arithmetic mean.

As described herein, in some instances, the antibody-drug conjugate may include a branched linker, where two drugs or active agents are attached to a branched linker. In certain embodiments, the two drugs or active agents attached to a branched linker are the same drug or active agent. For example, a first branch of a branched linker may be attached to a drug or an active agent and a second branch of the branched linker may be attached to the same drug or the same active agent as the first branch. In other embodiments, the two drugs or active agents attached to the branched linker are different drugs or active agents. For example, a first branch of a branched linker may be attached to a first drug or a first active agent and a second branch of the branched linker may be attached to a second drug or a second active agent different from the first drug or the first active agent attached to the first branch.

In some embodiments, where two different drugs or active agents are attached to the branched linker, the drugs or active agents may be selected from drugs and active agents that have a synergistic therapeutic effect. For example, in some instances, the use of two different drugs or active agents attached to the branched linker may provide a lower therapeutically effective concentration at which both payloads act, thereby increasing overall potency of the ADC.

In some embodiments, where two different drugs or active agents are attached to the branched linker, the drugs or active agents may be selected from drugs and active agents that provide an enhanced therapeutic benefit as compared to the use of the drugs or active agents separately, For example, the drugs or active agents may provide an increased effect on drug delivery of the ADC (e.g., some payloads, such as the iRGD peptide, can increase extravasation into tissues and augment tumor penetration).

In some embodiments, where two different drugs or active agents are attached to the branched linker, the drugs or active agents may be selected from drugs and active agents that use different mechanisms of action. In some cases, this may provide a decrease in tumor drug resistance by targeting multiple pathways. Examples of payload combinations can include, but are not limited to, cytotoxic drugs, immunomodulatory molecules to activate or inhibit immune cell populations, cytokines, hormones, chelating agents loaded with radioisotopes, and the like.

In some embodiments, where two different drugs or active agents are attached to the branched linker, the two different drugs or active agents are a topoisomerase inhibitor (e.g., belotecan) as described herein and an auristatin (e.g., MMAE) as described herein. In some embodiments, where two different drugs or active agents are attached to the branched linker, the two different drugs or active agents are a topoisomerase inhibitor (e.g., belotecan) as described herein and an iRGD peptide as described herein. In some embodiments, where two different drugs or active agents are attached to the branched linker, the two different drugs or active agents are an auristatin (e.g., MMAE) as described herein and an iRGD peptide as described herein. In some embodiments, where two different drugs or active agents are attached to the branched linker, the two different drugs or active agents are an auristatin (e.g., MMAE) as described herein and a kinase inhibitor (e.g., Sorafenib, Lapatinib, Gefitinib, and the like) as described herein. In some embodiments, where two different drugs or active agents are attached to the branched linker, the two different drugs or active agents are a topoisomerase inhibitor (e.g., belotecan) as described herein and a kinase inhibitor (e.g., Sorafenib, Lapatinib, Gefitinib, and the like) as described herein. In some embodiments, where two different drugs or active agents are attached to the branched linker, the two different drugs or active agents are an auristatin (e.g., MMAE) as described herein and a selective estrogen receptor modulator (e.g., Endoxifen) as described herein. In some embodiments, where two different drugs or active agents are attached to the branched linker, the two different drugs or active agents are a topoisomerase inhibitor (e.g., belotecan) as described herein and a selective estrogen receptor modulator (e.g., Endoxifen) as described herein.

Drugs to be conjugated to a polypeptide may be modified to incorporate a reactive partner for reaction with the polypeptide. Where the drug is a peptide drug, the reactive moiety (e.g., aminooxy or hydrazide) can be positioned at an N-terminal region, the N-terminus, a C-terminal region, the C-terminus, or at a position internal to the peptide. For example, an example of a method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy)propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl)carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexafluorophosphate), HBTU (O-benzotriazol-1-yl-N,N,N′,N′-tetramethyluronium hexafluorophosphate), TBTU (O-benzotriazol-1-yl-N,N,N′,N′-tetramethyluronium tetrafluoroborate), TSTU (O⁻(N-succinimidyl)-N,N,N′,N′-tetramethyluronium tetrafluoroborate), HATU (N-[(dimethylamino)-1-H-1,2,3-triazolo[4,5,6]-pyridin-1-ylmethylene]-N-methylmethanaminium hexafluorophosphate N-oxide), BOP-CI (bis(2-oxo-3-oxazolidinyl)phosphinic chloride), PyBOP ((1-H-1,2,3-benzotriazol-1-yloxy)-tris(pyrrolidino)phosphonium tetrafluorophopsphate), BrOP (bromotris(dimethylamino)phosphonium hexafluorophosphate), DEPBT (3-(diethoxyphosphoryloxy)-1,2,3-benzotriazin-4(3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group. Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (i.e., the accessibility of the amino acid residue to reaction with a reactive partner of interest) are of importance, Modifying reaction conditions to provide for optimal conjugation conditions is well within the skill of the ordinary artisan, and is routine in the art. Where conjugation is conducted with a polypeptide present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of polypeptides on a cell surface can be similar to those used in a ketone-azide reaction in modification of cells bearing cell-surface azides (see, e.g., U.S. Pat. No. 6,570,040).

Small molecule compounds containing, or modified to contain, an α-nucleophilic group that serves as a reactive partner with a compound or conjugate disclosed herein are also contemplated for use as drugs in the polypeptide-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

### Formulations

The conjugates of the present disclosure can be formulated in a variety of different ways. In general, where the conjugate is an antibody-drug conjugate, the conjugate is formulated in a manner compatible with the drug, the antibody, the condition to be treated, and the route of administration to be used.

In some embodiments, provided is a pharmaceutical composition that includes any of the conjugates of the present disclosure and a pharmaceutically acceptable excipient.

The conjugate (e.g., antibody-drug conjugate) can be provided in any suitable form, e.g., in the form of a pharmaceutically acceptable salt, and can be formulated for any suitable route of administration, e.g., oral, topical or parenteral administration. Where the conjugate is provided as a liquid injectable (such as in those embodiments where they are administered intravenously or directly into a tissue), the conjugate can be provided as a ready-to-use dosage form, or as a reconstitutable storage-stable powder or liquid composed of pharmaceutically acceptable carriers and excipients.

Methods for formulating conjugates can be adapted from those readily available. For example, conjugates can be provided in a pharmaceutical composition comprising a therapeutically effective amount of a conjugate and a pharmaceutically acceptable carrier (e.g., saline). The pharmaceutical composition may optionally include other additives (e.g., buffers, stabilizers, preservatives, and the like). In some embodiments, the formulations are suitable for administration to a mammal, such as those that are suitable for administration to a human.

### Methods of Treatment

The antibody-drug conjugates (ADCs) of the present disclosure find use in treatment of a condition or disease in a subject that is amenable to treatment by administration of the parent drug (i.e., the drug prior to conjugation to the antibody).

In some embodiments, provided are methods that include administering to a subject an amount (e.g., a therapeutically effective amount) of any of the conjugates of the present disclosure.

In certain aspects, provided are methods of delivering a drug to a target site in a subject, the method including administering to the subject a pharmaceutical composition including any of the conjugates of the present disclosure, where the administering is effective to deliver a therapeutically effective amount of the ADC to the target site in the subject.

In some instances, provided are methods of delivering a drug to a target site in a subject, the method including administering to the subject a pharmaceutical composition including any of the conjugates of the present disclosure, where the administering is effective to release a therapeutically effective amount of the drug from the conjugate at the target site in the subject. For example, as described herein, antibody-drug conjugates of the present disclosure can include a cleavable linker, such as an enzymatically cleavable linker that includes a first enzymatically cleavable moiety and a second enzymatically cleavable moiety. In some instances, the cleavable linker can be cleaved under appropriate conditions to separate or release the drug from the antibody at a desired target site of action for the drug. For example, the second cleavable moiety, which protects the first cleavable moiety from cleavage, may be cleaved in order to allow the first cleavable moiety to be cleaved, which results in cleavage of the cleavable linker into two or more portions, thus releasing the drug from the antibody-drug conjugate at a desired site of action.

In certain embodiments, the first cleavable moiety can be an enzymatically cleavable moiety. In some instances, the enzyme that facilitates cleavage of the first cleavable moiety is an enzyme that is administered to the subject to be treated (i.e., exogenous to the subject to be treated). For example, a first enzyme can be administered before, concurrently with, or after administration of an antibody-drug conjugate described herein.

In certain embodiments, the second cleavable moiety can be an enzymatically cleavable moiety. In some instances, the enzyme that facilitates cleavage of the second cleavable moiety is an enzyme that is administered to the subject to be treated (i.e., exogenous to the subject to be treated). For example, a second enzyme can be administered before, concurrently with, or after administration of an antibody-drug conjugate described herein. In certain embodiments, the first enzyme and the second enzyme are different enzymes.

In other instances, the first enzyme that facilitates cleavage of the first cleavable moiety is an enzyme that is present in the subject to be treated (i.e., endogenous to the subject to be treated). For instance, the first enzyme may be present at the desired site of action for the drug of the antibody-drug conjugate. The antibody of the antibody-drug conjugate may be specifically targeted to a desired site of action (e.g., may specifically bind to an antigen present at a desired site of action), where the desired site of action also includes the presence of the first enzyme. In some instances, the first enzyme is present in an overabundance at the desired site of action as compared to other areas in the body of the subject to be treated. For example, the first enzyme may be overexpressed at the desired site of action as compared to other areas in the body of the subject to be treated. In some instances, the first enzyme is present in an overabundance at the desired site of action due to localization of the first enzyme at a particular area or location. For instance, the first enzyme may be associated with a certain structure within the desired site of action, such as lysosomes. In some cases, the first enzyme is present in an overabundance in lysosomes as compared to other areas in the body of the subject. In some embodiments, the lysosomes that include the first enzyme, are found at a desired site of action for the drug of the antibody-drug conjugate, such as the site of a cancer or tumor that is to be treated with the drug. In certain embodiments, the first enzyme is a protease, such as a human protease enzyme (e.g., cathepsin B).

In certain embodiments, the second enzyme that facilitates cleavage of the second cleavable moiety is an enzyme that is present in the subject to be treated (i.e., endogenous to the subject to be treated). For instance, the second enzyme may be present at the desired site of action for the drug of the antibody-drug conjugate. The antibody of the antibody-drug conjugate may be specifically targeted to a desired site of action (e.g., may specifically bind to an antigen present at a desired site of action), where the desired site of action also includes the presence of the second enzyme. In some instances, the second enzyme is present in an overabundance at the desired site of action as compared to other areas in the body of the subject to be treated. For example, the second enzyme may be overexpressed at the desired site of action as compared to other areas in the body of the subject to be treated. In some instances, the second enzyme is present in an overabundance at the desired site of action due to localization of the second enzyme at a particular area or location. For instance, the second enzyme may be associated with a certain structure within the desired site of action, such as lysosomes. In some cases, the second enzyme is present in an overabundance in lysosomes as compared to other areas in the body of the subject. In some embodiments, the lysosomes that include the second enzyme, are found at a desired site of action for the drug of the antibody-drug conjugate, such as the site of a cancer or tumor that is to be treated with the drug. In certain embodiments, the second enzyme is a glucuronidase, a glycosidase, such as a galactosidase, a glucosidase, a mannosidase, a fucosidase, and the like.

Any suitable enzymes can be used for cleavage of the first cleavable moiety and the second cleavable moiety of the antibody-drug conjugates described herein. Other enzymes may also be suitable for use in cleavage of the first cleavable moiety and the second cleavable moiety of the antibody-drug conjugates described herein, such as but not limited to, enzymes from other vertebrates (e.g., primates, mice, rats, cats, pigs, quails, goats, dogs, rabbits, etc.).

In certain embodiments, the antibody-drug conjugate is substantially stable under standard conditions. By substantially stable is meant that the cleavable linker of the antibody-drug conjugate does not undergo a significant amount of cleavage in the absence of a first enzyme and a second enzyme as described above. For example, as described above, the second cleavable moiety can protect the first cleavable moiety from being cleaved, and as such the cleavable linker of the antibody-drug conjugate does not undergo a significant amount of cleavage in the absence of a second enzyme as described above. For instance, the cleavable linker of the antibody-drug conjugate may be substantially stable such that 25% or less of the antibody-drug conjugate is cleaved in the absence of the first enzyme and/or second enzyme, such as 20% or less, or 15% or less, or 10% or less, or 5% or less, or 4% or less, or 3% or less, or 2% or less, or 1% or less. In some cases, the antibody-drug conjugate is substantially stable such that the cleavable linker of the antibody-drug conjugate does not undergo a significant amount of cleavage in the absence of the first enzyme and/or second enzyme, but can be cleaved when in the presence of the first enzyme and the second enzyme. For example, the antibody-drug conjugate can be substantially stable after administration to a subject. In some cases, the antibody-drug conjugate is substantially stable after administration to a subject, and then, when the antibody-drug conjugate is in the presence of the second enzyme at a desired site of action, the second cleavable moiety can be cleaved from the cleavable linker, thus exposing the first cleavable moiety to subsequent cleavage by the first enzyme, which in turn releases the drug at the desired site of action. In certain embodiments, after administration to a subject the antibody-drug conjugate is stable for an extended period of time in the absence of the first enzyme and/or second enzyme, such as 1 hr or more, or 2 hrs or more, or 3 hrs or more, or 4 hrs or more, or 5 hrs or more, or 6 hrs or more, or 7 hrs or more, or 8 hrs or more, or 9 hrs or more, or 10 hrs or more, or 15 hrs or more, or 20 hrs or more, or 24 hrs (1 day) or more, or 2 days or more, or 3 days or more, or 4 days or more, or 5 days or more, or 6 days or more, or 7 days (1 week) or more. In certain embodiments, the antibody-drug conjugate is stable at a range pH values for an extended period of time in the absence of the first enzyme and/or second enzyme, such as at a pH ranging from 2 to 10, or from 3 to 9, or from 4 to 8, or from 5 to 8, or from 6 to 8, or from 7 to 8.

As described above, antibody-drug conjugates of the present disclosure find use in treatment of a condition or disease in a subject that is amenable to treatment by administration of the parent drug. By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject to be treated can be one that is in need of therapy, where the subject to be treated is one amenable to treatment using the parent drug. Accordingly, a variety of subjects may be amenable to treatment using the antibody-drug conjugates disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees and monkeys).

The amount of antibody-drug conjugate administered can be initially determined based on guidance of a dose and/or dosage regimen of the parent drug. In general, the antibody-drug conjugates can provide for targeted delivery and/or enhanced serum half-life of the bound drug, thus providing for at least one of reduced dose or reduced administrations in a dosage regimen. Thus, the antibody-drug conjugates can provide for reduced dose and/or reduced administration in a dosage regimen relative to the parent drug prior to being conjugated in an antibody-drug conjugate of the present disclosure.

Furthermore, as noted above, because the antibody-drug conjugates can provide for controlled stoichiometry of drug delivery, dosages of antibody-drug conjugates can be calculated based on the number of drug molecules provided on a per antibody-drug conjugate basis.

In some embodiments, multiple doses of an antibody-drug conjugate are administered. The frequency of administration of an antibody-drug conjugate can vary depending on any of a variety of factors, e.g., severity of the symptoms, condition of the subject, etc. For example, in some embodiments, an antibody-drug conjugate is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Methods of Treating Cancer

The present disclosure provides methods that include delivering a conjugate of the present disclosure to an individual having a cancer. For example, the method may include administering to the subject a therapeutically effective amount of a pharmaceutical composition comprising a conjugate of the present disclosure, where the administering is effective to treat cancer in the subject. The methods are useful for treating a wide variety of cancers, including, but not limited to breast, ovarian, colon, lung, stomach, and pancreatic cancer. In the context of cancer, the term “treating” includes one or more (e.g., each) of: reducing growth of a solid tumor, inhibiting replication of cancer cells, reducing overall tumor burden, and ameliorating one or more symptoms associated with a cancer.

Carcinomas that can be treated using a subject method include, but are not limited to, colon carcinoma, colorectal carcinoma, gastric carcinoma, lung carcinoma, including small cell carcinoma and non-small cell carcinoma of the lung, pancreatic carcinoma, breast carcinoma, ovarian carcinoma, prostate carcinoma, adenocarcinoma, cystadenocarcinoma, medullary carcinoma, renal cell carcinoma, ductal carcinoma in situ or bile duct carcinoma, choriocarcinoma, seminoma, embryonal carcinoma, cervical carcinoma, uterine carcinoma, testicular carcinoma, and epithelial carcinoma, etc.

In certain aspects, provided are methods of treating cancer in a subject, such methods including administering to the subject a therapeutically effective amount of a pharmaceutical composition including any of the conjugates of the present disclosure, where the administering is effective to treat cancer in the subject.

### Methods of Producing a Conjugate

The present disclosure provides methods for producing a conjugate as described herein. In certain embodiments, the method includes:
contacting an aldehyde-tagged peptide with a payload comprising a 1,2-aminothiol group under conditions to produce a conjugate of formula (I):

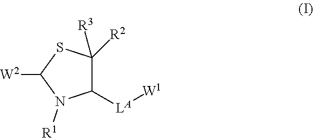

wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker;
W¹ is the payload; and
W² is the peptide.

Regarding formula (I) recited above, the substituents R¹, R², R³, L^{A}, W¹ and W² are as described above in relation to the conjugates of formula (I). Similarly, regarding the first linker L^{A} and the second linker L^{B}, the T¹, T², T³, T⁴, T⁵, T⁶, V¹, V², V³, V⁴, V⁵ and V⁶, and T⁷, T⁸, T⁹, T¹⁰, T¹¹, T¹², V⁷, V⁸, V⁹, V¹⁰, V¹¹ and V¹² substituents are as described above in relation to the conjugates of formula (I).

In order to make the conjugate, an amino acid residue of the polypeptide may be modified and then coupled to a payload (e.g., one or more drugs or active agents) attached to the 1,2-aminothiol group as described herein. In certain embodiments, an amino acid residue of the polypeptide (e.g., antibody) is a cysteine or serine residue that is converted to an fGly residue, as described herein. In certain embodiments, the converted amino acid residue (e.g., fGly residue) is conjugated to the payload (e.g., one or more drugs or active agents) containing a 1,2-aminothiol group as described herein to provide a conjugate of the present disclosure, where the one or more drugs or active agents are conjugated to the polypeptide through a thiazolidine conjugation moiety. As described herein, the 1,2-aminothiol group may be present as part of a linker attached to the one or more drugs or active agents.

As described herein, in methods of producing the conjugate, an aldehyde-tagged peptide (e.g., a polypeptide or antibody containing one or more fGly residues) may be conjugated to a payload by reaction of the fGly with the payload (e.g., a compound containing a 1,2-aminothiol group, as described herein). For example, an aldehyde-tagged peptide (e.g., a polypeptide or antibody containing one or more fGly residues) may be contacted with a reactive partner containing an aldehyde-reactive group and a payload, such as a 1,2-aminothiol group, under conditions suitable to provide for conjugation of the compound containing the 1,2-aminothiol group to the aldehyde-tagged peptide, thus attaching the payload to the peptide. Reaction of the aldehyde group in the fGly residue of the peptide (antibody) with the 1,2-aminothiol group of the linker attached to the payload produces a thiazolidine conjugation moiety (e.g., as shown in the conjugate of formula (I) herein).

In some instances, the reactive partner may include a 1,2-aminothiol group as described herein. For example, one or more drugs or active agents may be attached to a 1,2-aminothiol group through a linker to provide a payload having an aldehyde-reactive group. In some cases, the one or more drugs or active agents are attached to a 1,2-aminothiol group, such as covalently attached to a 1,2-aminothiol group, where each drug or active agent is attached through a corresponding linker to the 1,2-aminothiol group.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

The present invention stems from a number of surprising findings related to thiazolidine chemistry. Specifically, introducing gem-substituents at position 1 of 1,2-aminothiols dramatically increased the efficiency of the thiazolidine conjugation reaction (FIG. 6, path A) and decreased the degree of the undesired oxidative dimerization side-reaction (FIG. 6, path B), compared to the 1,1-unsubstituted congeners. Moreover, thiazolidine conjugates resulting from gem-disubstituted 1,2-aminothiols showed superior stability under physiologically relevant conditions compared to their unsubstituted analogs and therefore exhibited excellent in vivo stability, leading to more tolerable and efficacious ADCs. Interestingly, it was also determined that introducing a substituent at the nitrogen atom of 1,2-aminothiol which led to the secondary amine analog significantly decreased the rate of reactivity towards an aldehyde group and unfavorably altered the stability of the resulting thiazolidine moiety. This finding further highlighted the uniqueness of 1,1-dialkyl 1,2-aminothiols disclosed in the present invention with respect to their utility in conjugating aldehyde-tagged macromolecules.

### Material and Methods

### General Information

Synthetic reagents were purchased from Sigma-Aldrich, Acros, AK Scientific, or other commercial sources and were used without purification. Anhydrous solvents were obtained from commercial sources in sealed bottles. Compound 1 was obtained commercially from Shanghai Medicilon and used without purification. Cytotoxic payloads belotecan HCl (4) and MMAE (2) were purchased from commercial sources. In all cases, solvent was removed under reduced pressure with a Buchi Rotovapor R-114 equipped with a Buchi V-700 vacuum pump. Column chromatography was performed using a Biotage chromatography system. Preparative HPLC purifications were performed using Waters preparative HPLC unit equipped with Phenomenex Kinetex 5 mm EVO C18 150×21.2 mm column. HPLC analyses were conducted on an Agilent 1100 Series Analytical HPLC equipped with a Model G1322A Degasser, Model G1311A Quarternary Pump, Model G1329A Autosampler, Model G1314 Variable Wavelength Detector, Agilent Poroshell 120 SB C18, 4.6 mm×50 mm column at room temperature using a 10-100% gradient of water and acetonitrile containing 0.05% trifluoroacetic acid. HPLCs were monitored at 254 or 205 nm. Low-resolution mass spectra (LRMS) were acquired on Agilent Technology 6120 Quadrupole LC/MS, equipped with Agilent 1260 Infinity HPLC system, G1314 variable wavelength detector, and Agilent Poroshell 120 SB C18, 4.6 mm×50 mm column at room temperature using 10-100% gradient of water and acetonitrile containing 0.1% formic acid.

### Synthesis of drug-linkers

### Synthesis of (25,35,45,5R,65)-6-(2-((S)-2-((S)-2-((R)-2-amino-3-mercaptopropanamido)-3-methylbutanamido)propanamido)-5-((S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (5)

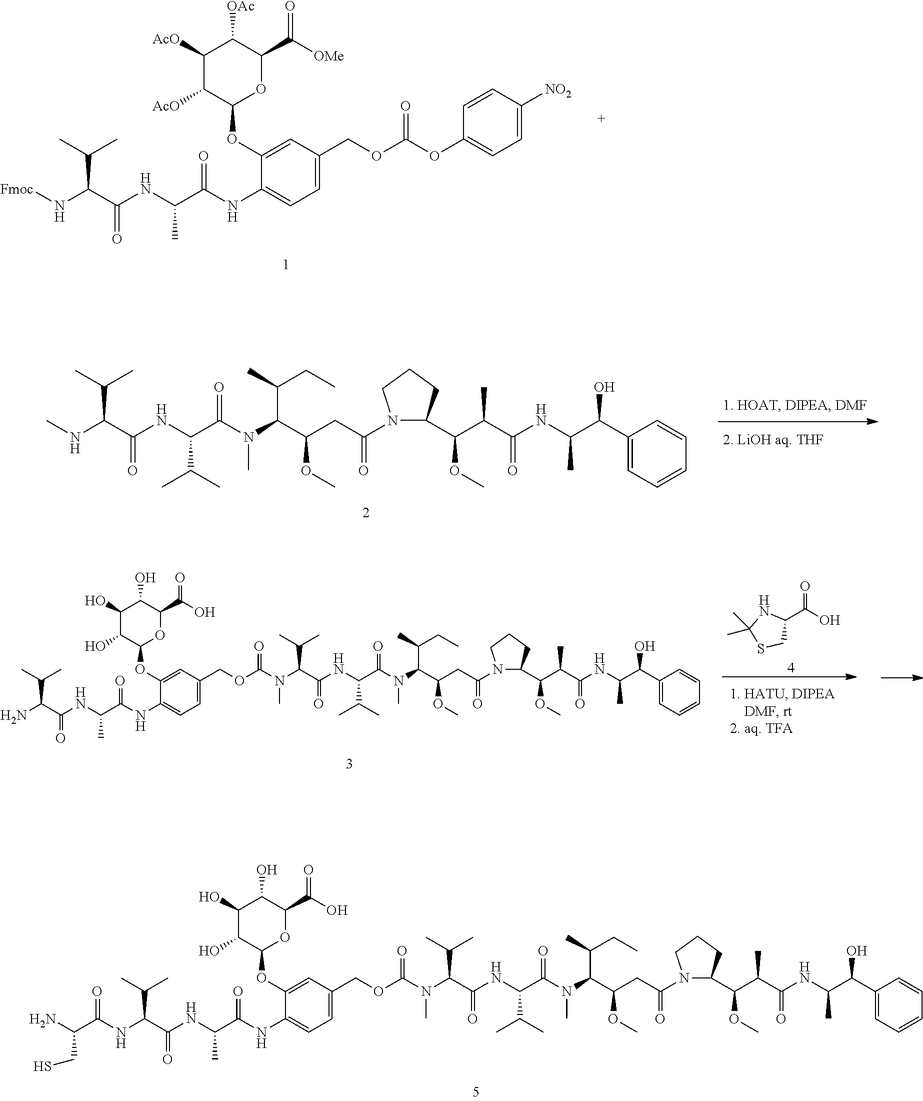

In a 20 mL glass vial were combined monomethyl auristatin E (MMAE, 2, 720 mg, 1.0 mmol), 5 mL of anhydrous DMF, and 0.35 mL of DIPEA (2.0 mmol) at room temperature. The resulting mixture was stirred and treated with PNP carbonate 1 (1014 mg, 1.0 mmol) as a solid in a few small portions, followed by the addition of HOAt (136 mg, 1.0 mmol) in one portion at room temperature. Reaction mixture was stirred for 6 h until reaction was judged complete (HPLC). Reaction mixture was poured into 30 mL of water, and the resulting white precipitate was collected by filtration, washed with water, and dried briefly under high vacuum to give 1.87 g of crude coupling product as a yellow solid, which was taken to the next step without purification.

A solution of crude intermediate (1.87 g) in 15 mL of THF was cooled down to 0° C. in an ice bath and treated slowly with 1 M aqueous lithium hydroxide solution (3 mL). The reaction mixture was stirred at 0° C. for 3 hours, then warmed up to ambient temperature and treated with another 3 mL of 1 M aqueous lithium hydroxide. The resulting mixture was stirred at room temperature for 3 hours until hydrolysis was found complete (HPLC), then quenched by adding 1 M aqueous HCl solution to pH 7. The reaction mixture was then concentrated under reduced pressure and washed with 10 mL of MTBE. The aqueous layer was purified by reversed-phase chromatography (C18 column, 0-40% acetonitrile-water with 0.05% TFA). Pure product fractions were combined, concentrated under reduced pressure, and lyophilized to give compound 3 as a white powder (735 mg, 0.60 mmol, 60% yield over 2 steps). LRMS (ESI): m/z 1229.7 [M⁺H]⁺, Calcd for C₆₁H₉₆N₈O₁₈ m/z 1229.7.

To a solution of acetonide 4 (10 mg, 62 μmol) in anhydrous DMF (2 mL) was added HATU (24 mg, 62 μmol) and DIPEA (22 μL, 124 μmol) at RT. The mixture was stirred for 15 seconds and immediately combined with a solution of compound 3 (25 mg, 20 μmol) in anhydrous DMF (0.5 mL). After 30 minutes, reaction mixture was purified by reversed-phase prep HPLC (C18 column, 0-70% acetonitrile-water with 0.05% TFA). Pure fractions were combined and concentrated under reduced pressure to remove acetonitrile. The residual aqueous solution was diluted with 1 mL of 10% TFA in water. The resulting mixture was then purged with dinitrogen, sealed, kept at RT overnight, followed by direct lyophilization to give pure product 5 (13 mg, 10 μmol, 50% yield) as a white foam. LRMS (ESI): m/z 1332.7 [M⁺H]⁺, Calcd for C₆₄H₁₀N₉O₁₉S m/z 1332.7.

### Synthesis of (R)-2,2-dimethylthiazolidine-4-carboxylic acid (4)

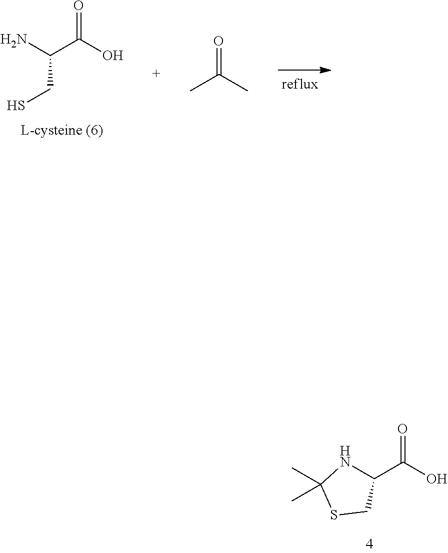

A solution of L-cysteine 6 (1.0 g, 8.25 mmol) in acetone (100 mL) was refluxed at 55° C.-60° C. until all solids were dissolved (^{˜}5 h). The reaction was allowed to cool to room temperature. Crystallization occurred overnight. The solids were collected by filtration and washed with minimal cold acetone and dried on air to give acetonide 4 as white needles (1.1 g, 6.8 mmol, 83% yield).

### Synthesis of (25,35,45,5R,6S)-6-(2-((S)-2-((S)-2-((S)-2-amino-3-mercapto-3-methylbutanamido)-3-methylbutanamido)propanamido)-5-((S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (8)

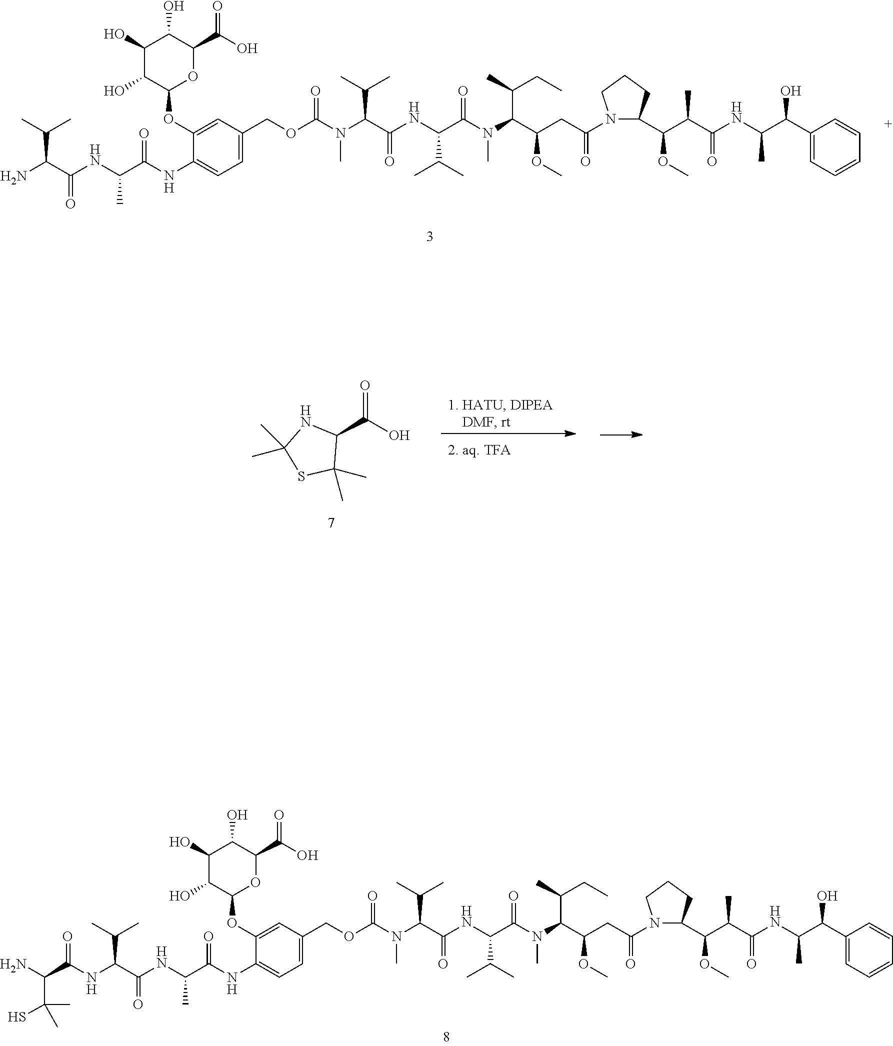

To a solution of acetonide 7 (2 mg, 10 μmol) in anhydrous DMF (2 mL) were added HATU (4 mg, 10 μmol) and DIPEA (4 uL, 20 μmol) at RT. The mixture was stirred for 30 seconds before adding a solution of compound 3 (13 mg, 10 μmol) in DMF (0.5 mL). Reaction mixture was stirred at RT for 1 h and then directly purified by reversed-phase prep HPLC (C18, acetonitrile-water with 0.05% TFA). Pure fractions were combined and concentrated under reduced pressure to remove acetonitrile. The resulting solution was diluted with 1 mL of 10% (v/v) TFA in water. The mixture was then purged with N₂, sealed, and kept at RT overnight, followed by direct lyophilization to obtain pure product 8 (11 mg, 8 μmol, 80% yield) as a white foam. LRMS (ESI): m/z 1360.7 [M⁺H]⁺, Calcd for C₆₆H₁₀₅N₉O₁₉S m/z 1360.7.

### Synthesis of (S)-2,2,5,5-tetramethylthiazolidine-4-carboxylic acid (7)

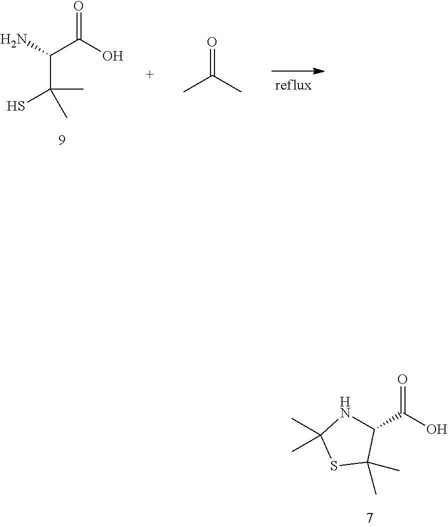

A solution of D-penicillamine 9 (1.0 g, 6.7 mmol) in acetone (100 mL) was refluxed at 55° C.-60° C. until the majority of solids dissolved (^{˜}9 h). The remaining solids were filtered, and the filtrate was allowed to cool to room temperature to start crystallization and placed in a −20° C. freezer overnight. The resulting precipitate was collected by filtration, washed with minimal cold acetone, and dried on air to give acetonide 7 as white sharp needles (0.90 g, 4.8 mmol, 72% yield) and used directly for the next step.

### Synthesis of (25,35,45,5R,65)-6-(2-((S)-2-((S)-2-((R)-2-amino-3-mercaptopropanamido)-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-1l-yl)ethyl)(isopropyl)carbamoyl)oxy)methyl)phenoxy)-3,4,5-trihydroxytetrahydro-2H-pyran-2-carboxylic acid (12)

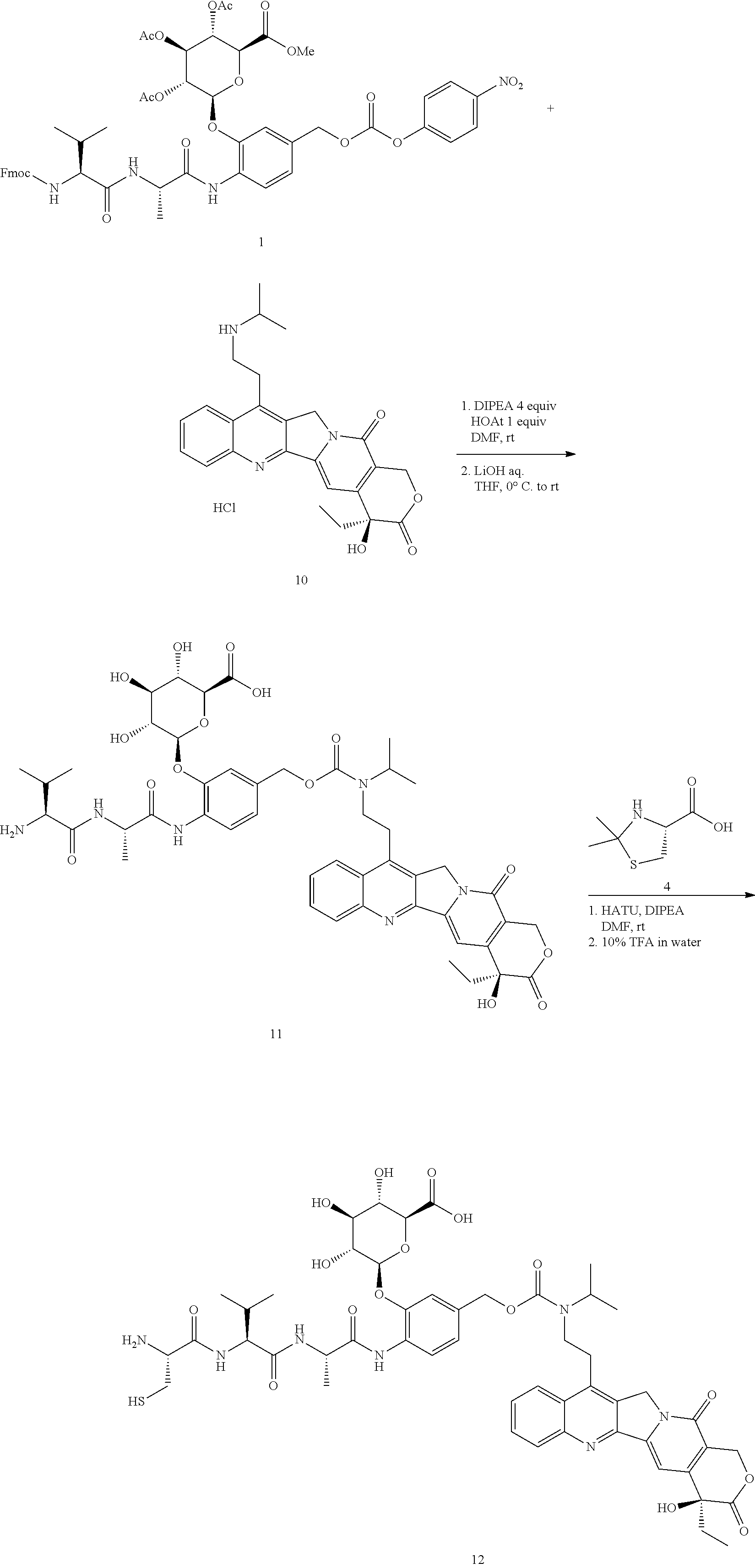

Belotecan hydrochloride 10 (2.35 g, 5.0 mmol) was suspended in a mixture of 30 mL of anhydrous DMF and 1.75 mL of DIPEA (10 mmol). The resulting mixture was stirred and treated with HOAt (0.68 g, 5 mmol), followed by PNP-carbonate 1 (5.1 g, 5 mmol) in a few small portions at room temperature. Reaction mixture was stirred at RT for 8 hours until starting materials were judged fully consumed based on HPLC analysis. The mixture was poured onto 300 mL of ice with vigorous stirring, the resulting yellowish precipitate was collected by filtration, washed with 30 mL of cold water twice, dried on air overnight to give 6.7 g of crude coupling product as a light-yellow powder which was taken to the next step without purification.

A solution of crude coupling intermediate (6.7 g) in 30 mL of THF was cooled down to 0° C. in an ice bath and treated slowly with 2 M aqueous lithium hydroxide solution (10 mL). Reaction mixture was stirred at 0° C. for 1 hour, then another 10 mL of 2 M LiOH solution was added and stirring continued for 15 minutes before warming the reaction mixture to room temperature and adding another 10 mL of 2 M lithium hydroxide. The resulting mixture was stirred for 1 hour at room temperature, then quenched by adding 2 M aqueous HCl solution to pH ^{˜}2-3 and let stir for 30 minutes. The mixture was transferred to a separatory funnel and washed with MTBE (2×50 mL). The aqueous layer was separated, then directly loaded on a C18 column, and eluted with 0-40% CH₃CN—H₂O with 0.05% TFA. Pure fractions were combined, concentrated under reduced pressure to ˜70 mL volume, and lyophilized to give 3.4 g of product 11 (3.6 mmol, 72% yield over 2 steps) as a bright-yellow fluffy powder. LRMS (ESI): m/z 945.4 [M⁺H]⁺, Calcd for C₄₇H₅₆N₆O₁₅ m/z 945.4.

To a solution of acetonide 4 (100 mg, 62 μmol) in DMF (5 mL) were added HATU (236 mg, 62 μmol) and DIPEA (216 μL, 124 μmol) at RT. The mixture was stirred for 15 seconds before adding a solution of compound 11 (196 mg, 20 μmol) in DMF (1 mL). After 1 h, reaction mixture was purified by reversed-phase prep HPLC (0-70% acetonitrile with 0.05% TFA). Pure fractions were combined and concentrated under reduced pressure to remove acetonitrile. The resulting aqueous solution was diluted with 1 mL of 10% (v/v) TFA in water. The mixture was then purged with nitrogen, sealed, kept at RT overnight, followed by direct lyophilization to afford compound 12 (100 mg, 9.5 μmol, 48% yield) as a yellow foam. LRMS (ESI): m/z 1048.4 [M⁺H]⁺, Calcd for C₅₀H₆₁N₇O₁₆S m/z: 1048.4.

### Preparation of (25,35,45,5R,65)-6-(2-((S)-2-((S)-2-((R)-2-amino-3-mercapto-3-methylbutanamido)-3-methylbutanamido)propanamido)-5-((((2-((S)-4-ethyl-4-hydroxy-3,14-dioxo-3,4,12,14-tetrahydro-1 H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (13)

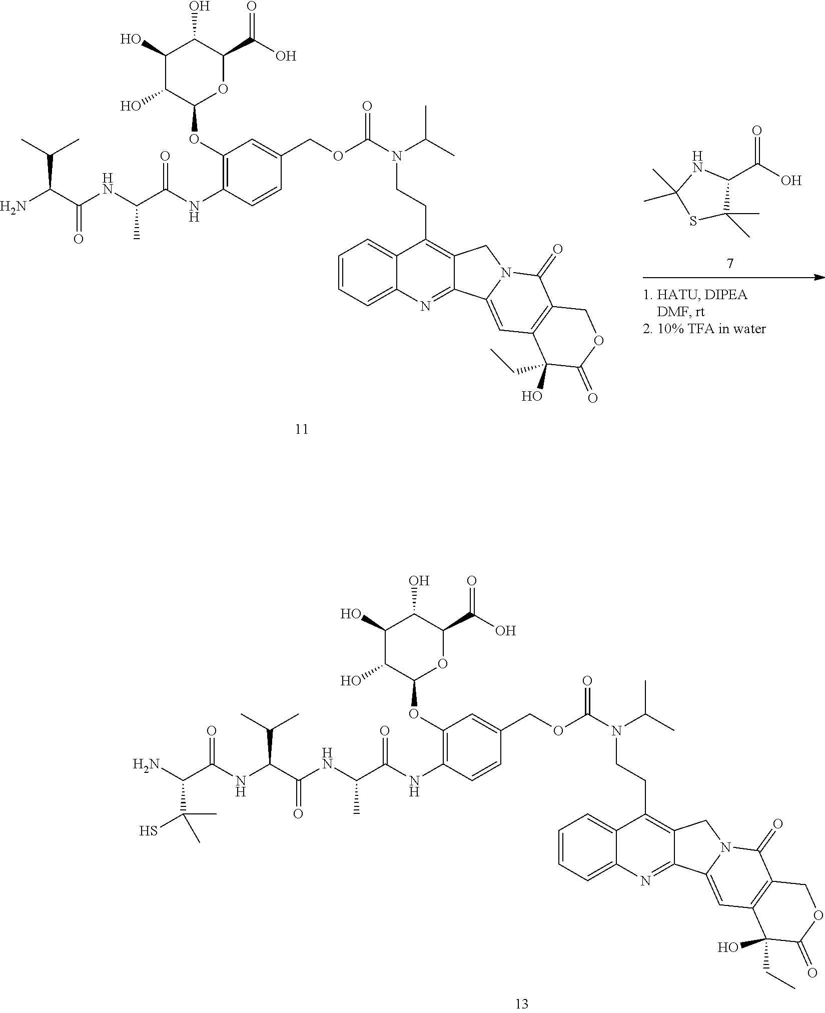 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](N)C(C)C)c(O[C@@H]2O[C@H](C(=O)O)[C@@H](O)[C@H](O)[C@H]2O)c1)C(C)C ; CC1(C)N[C@H](C(=O)O)C(C)(C)S1 ; CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@@H](N)C(C)(C)S)C(C)C)c(O[C@@H]2O[C@H](C(=O)O)[C@@H](O)[C@H](O)[C@H]2O)c1)C(C)C]

To a solution of acetonide 7 (20 mg, 106 μmol) in anhydrous DMF (5 mL) was added HATU (40 mg, 106 μmol) and DIPEA (37 μL, 212 μmol) at RT. The mixture was stirred for 30 seconds and combined with a solution of compound 11 (100 mg, 106 μmol) in anhydrous DMF (0.5 mL). After 1h, reaction mixture was directly purified by reversed-phase prep HPLC (0-70% acetonitrile with 0.05% TFA). Pure fractions were combined and concentrated under reduced pressure to remove acetonitrile. The resulting aqueous solution was diluted with 1 mL of 10% (v/v) TFA in water. The mixture was then purged with N₂, sealed, kept at RT overnight, and then directly lyophilized to give compound 13 (105 mg, 98 μmol, 92% yield) as a yellow foam. LRMS (ESI): m/z 1076.4 [M⁺H]⁺, Calcd for C₅₂H₆₅N₇O₁₆S m/z: 1076.4.

### Synthesis of (25,35,45,5R,65)-6-(5-((((2-((S)-4-ethyl-4-hydroxy-3,14-dioxo-3,4,12,14-tetrahydro-1H-pyrano[3,4′:6,7]indolizino[1,2-b]quinolin-11-yl)ethyl)(isopropyl)carbamoyl)oxy)methyl)-2-((S)-2-((S)-3-methyl-2-((R)-2,2,5,5-tetramethylthiazolidine-4-carboxamido)butanamido)propanamido)phenoxy)-3,4,5-trihydroxytetrahydro-2H-pyran-2-carboxylic acid (14)

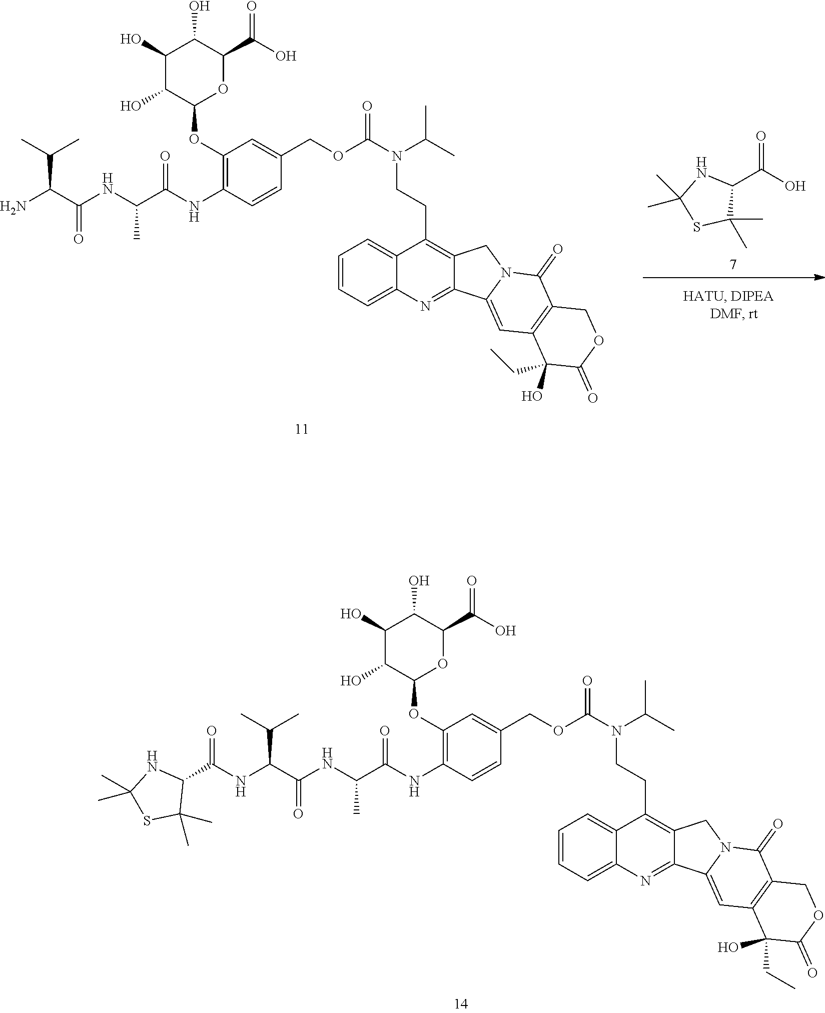 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](N)C(C)C)c(O[C@@H]2O[C@H](C(=O)O)[C@@H](O)[C@H](O)[C@H]2O)c1)C(C)C ; CC1(C)N[C@H](C(=O)O)C(C)(C)S1 ; CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H]2NC(C)(C)SC2(C)C)C(C)C)c(O[C@@H]2O[C@H](C(=O)O)[C@@H](O)[C@H](O)[C@H]2O)c1)C(C)C]

To a solution of acetonide 7 (20 mg, 106 μmol) in DMF (5 mL) was added HATU (40 mg, 106 μmol) and DIPEA (37 μL, 212 μmol) at rt. The mixture was stirred for 30 seconds before adding a solution of compound 11 (100 mg, 106 μmol) in DMF (0.5 mL). After 30 min, reaction mixture was purified by reversed-phase prep HPLC (0-70% acetonitrile with 0.1% formic acid). Pure fractions were combined and lyophilized to give compound 14 (106 mg, 95 μmol, 90% yield) as a yellow foam. Based on HPLC analysis, approximately 10% of acetonide was hydrolyzed during purification. LRMS (ESI): m/z 1116.5 [M⁺H]⁺, Calcd for C₅₅H₇₀N₇O₁₆S m/z: 1116.5.

### Synthesis of (25,35,45,5R,65)-6-(5-((((2-((S)-4-ethyl-4-hydroxy-3,14-dioxo-3,4,12,14-tetrahydro-1H-pyrano[3,4′:6,7]indolizino[1,2-b]quinolin-11-yl)ethyl)(isopropyl)carbamoyl)oxy)methyl)-2-((3R,16S,19S)-16-isopropyl-3-(mercaptomethyl)-19-methyl-4,14,17-trioxo-8,11-dioxa-2,5,15,18-tetraazaicosan-20-amido)phenoxy)-3,4,5-trihydroxytetrahydro-2H-pyran-2-carboxylic acid (19)

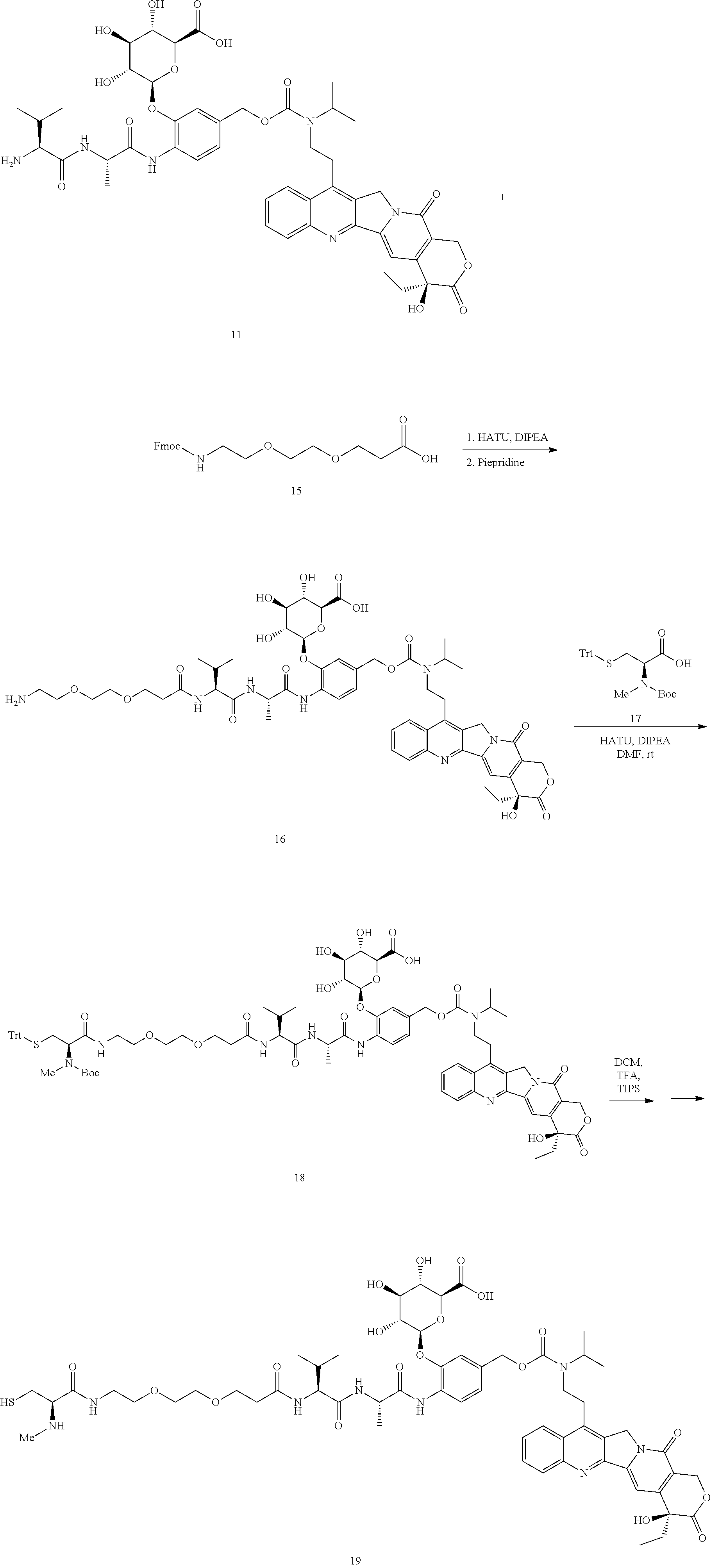

To a solution of compound 15 (40 mg, 0.1 mmol) in DMF (3 mL) were added HATU (38 mg, 0.1 mmol) and DIPEA (52 μL, 0.3 mmol) at room temperature. Reaction mixture was stirred for 10 minutes and combined with a solution of compound 11(90 mg, 95 μmol) in DMF (0.5 mL). After 1 h, piperidine (0.2 mL, 2 mmol) was added directly to the reaction mixture at RT in one shot. After 30 min, reaction mixture was purified by reversed-phase prep HPLC (0-70% acetonitrile with 0.05% TFA). Pure fractions were combined and lyophilized to afford compound 16 (94 mg, 90% yield) as a yellow foam. LRMS (ESI): m/z 1104.5 [M⁺H]⁺, Calcd for C₅₄H₇₀N₇O₁₈ m/z: 1104.5.

To a solution of compound 17 (8 mg, 16 μmol) in DMF (2 mL) were added HATU (6 mg, 16 μmol) and DIPEA (8 μL, 50 μmol) at room temperature. The reaction mixture was stirred for 10 minutes and combined with a solution of compound 16 (18 mg, 16 μmol) in DMF (0.5 mL). After 30 min, reaction mixture was purified by reversed-phase prep HPLC (0-70% acetonitrile with 0.05% TFA). Pure fractions were combined and lyophilized to give compound 18 (10 mg, 6 μmol, 38% yield) was obtained as a yellow foam. LRMS (ESI): m/z 1563.7 [M⁺H]⁺, Calcd for C₈₂H₉₉N₈O₂₁S m/z: 1563.7.

Compound 18 (10 mg, 6 μmol) and triisopropylsilane (2 mg, 13 μmol) were dissolved in a DCM-TFA mixture (1:1, 1 mL). The resulting solution was allowed to stand at room temperature for 10 minutes, then solvents were removed under vacuum and the residue was purified by reversed-phase prep HPLC (0-50% acetonitrile-water, 0.05% TFA). Pure fractions were combined, solvents were removed in vacuum and lyophilized to give compound 19 as a yellow foam (6 mg, 5 μmol, 83% yield). LRMS (ESI): m/z 1221.5, [M⁺H]⁺, Calcd for C₅₋₈H₇₇N₈O₁₉S m/z 1221.5.

### Example 2

### Stability of 12 and 13 Towards Oxidation in Stock DMA Solutions

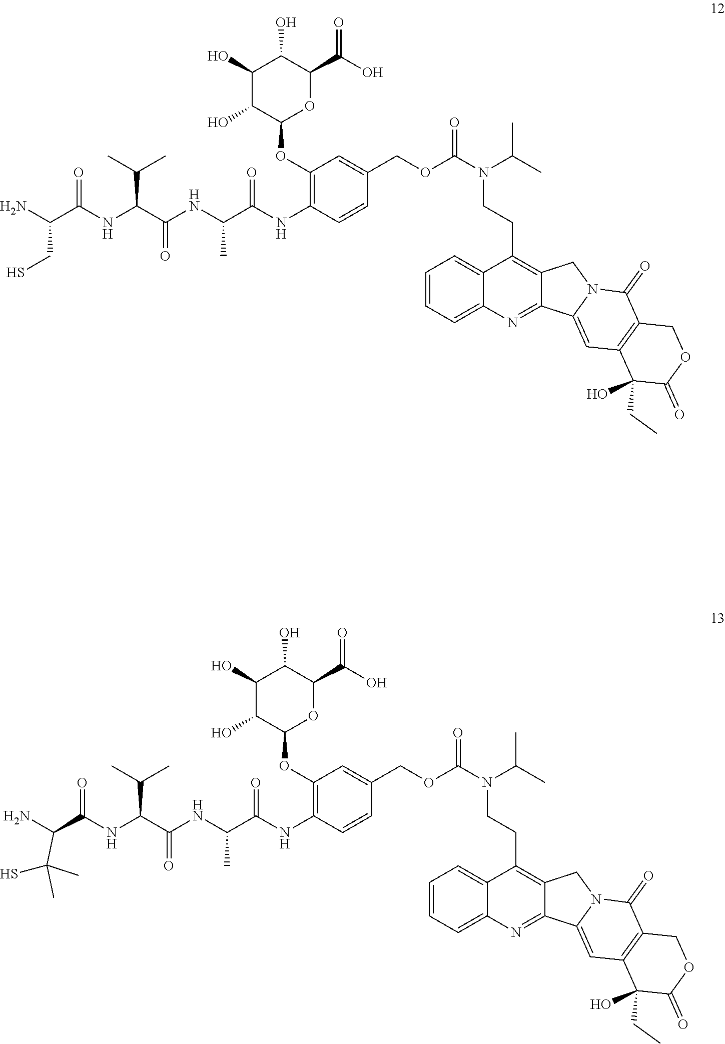 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@@H](N)CS)C(C)C)c(O[C@@H]2O[C@H](C(=O)O)[C@@H](O)[C@H](O)[C@H]2O)c1)C(C)C ; CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](N)C(C)(C)S)C(C)C)c(O[C@@H]2O[C@H](C(=O)O)[C@@H](O)[C@H](O)[C@H]2O)c1)C(C)C]

Compounds 12 and 13 were dissolved in DMA to final concentration of 50 mM in 1.5 mL Eppendorf tubes. Samples were immediately placed at −20° C. and stored for 7 days. At this point, LC-MS samples were prepared by diluting the DMA sample solution to 1 mM with pH 5.5 citrate buffer. LCMS was used to determine % oxidized to disulfide dimer. DMA samples of 12 and 13 were then placed at RT on a bench for 24 h, followed by LCMS analysis as described above.

### Results:

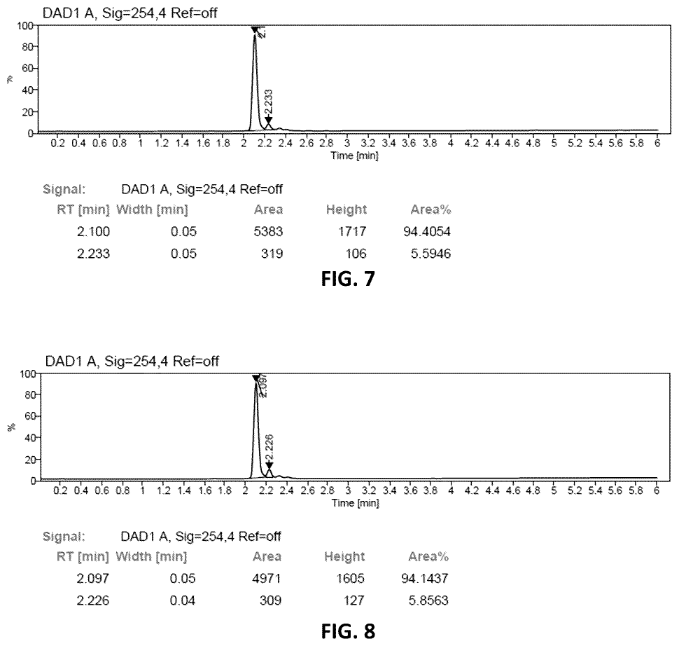

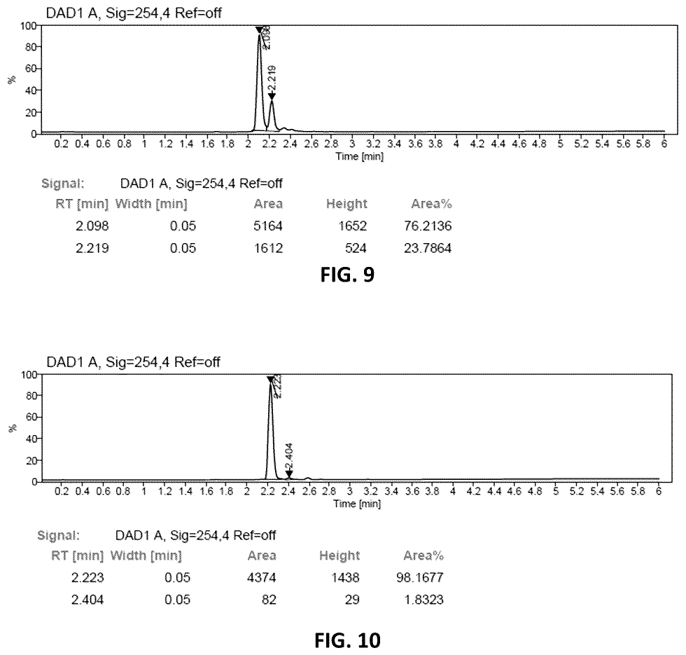

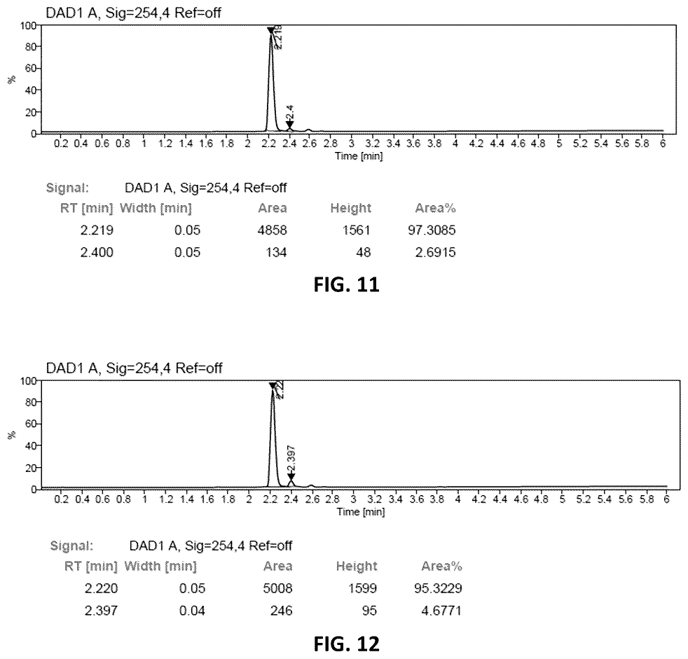

HPLC traces of 12 and 13 at TO are shown in FIG. 7 and FIG. 10, respectively. Both 12 and 13 showed minimal oxidation at −20° C. for 7 days (FIG. 8 for 12 and FIG. 11 for 13). At room temperature, 12 showed 25% oxidation after 24 h (FIG. 9), while 13 remained mostly intact (˜2% oxidation, FIG. 12).

### Example 3

### Stability of Benzyloxyacetaldehyde (BOAA) Conjugates 20 and 21 Under Conjugation Conditions

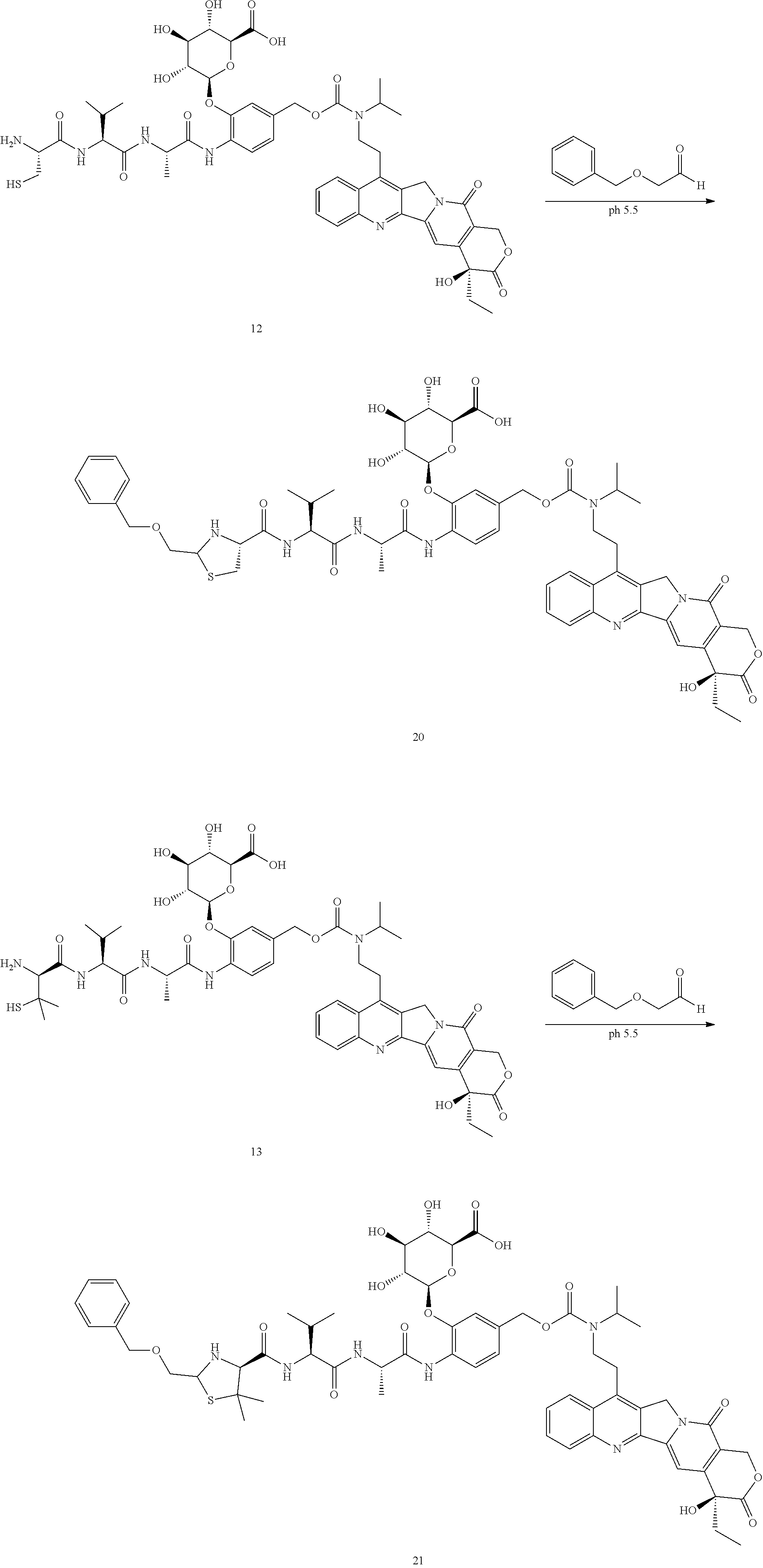

### Procedure:

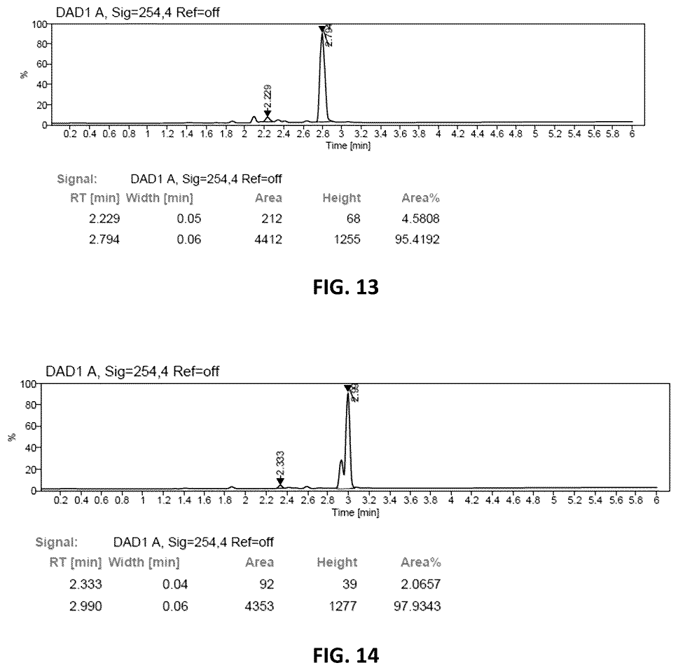

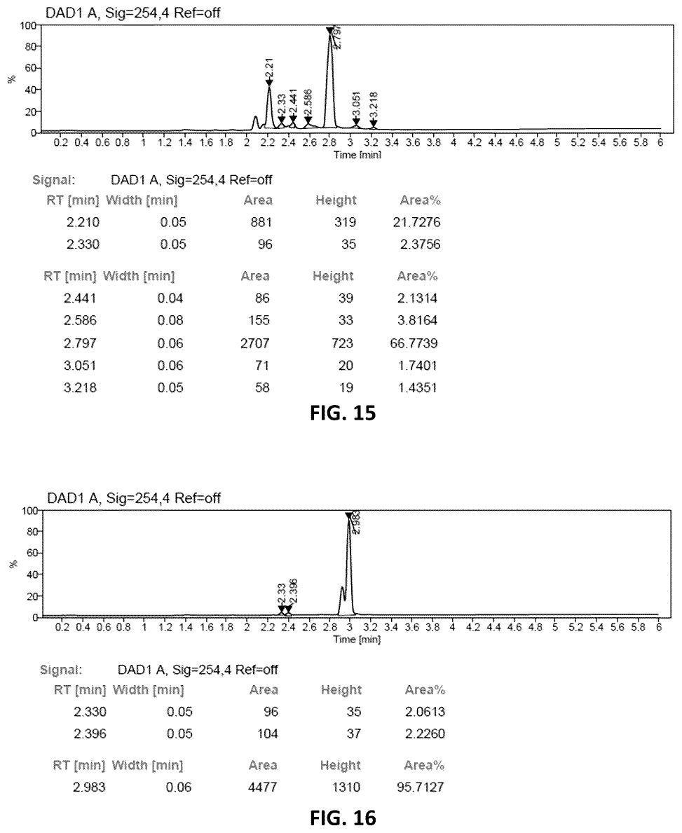

Stock solutions of 12 and 13 (50 mM in DMA, 1 NL) were diluted with 50 μL of pH 5.5 buffer (20 mM sodium citrate, 50 mM NaCl). To this diluted solution were added 1 μL of benzyloxyacetaldehyde (BOAA) solution in DMA (100 mM) at ambient temperature. After 30 min, formation of BOAA conjugates 20 and 21 were complete based on HPLC analysis (FIG. 13 and FIG. 14). Reaction mixtures were left at RT for 7 days and then analyzed again by HPLC (FIG. 15 for 20 and FIG. 16 for 21).

### Results:

Minimal changes were observed for conjugate 21 (FIG. 16), indicating high stability of thiazolidine ring, derived from D-penicillamine. In contrast, formation of the corresponding disulfide oxidized product was observed for conjugate 20 after 7 days, based on LCMS analysis (FIG. 15), which indicated that hydrolysis of thiazolidine 20 to revert to 12 had occurred, followed by oxidation.

### Example 4

Test Conjugation of N-Methyl L-Cysteine Construct 19 with Benzyloxyacetaldehyde

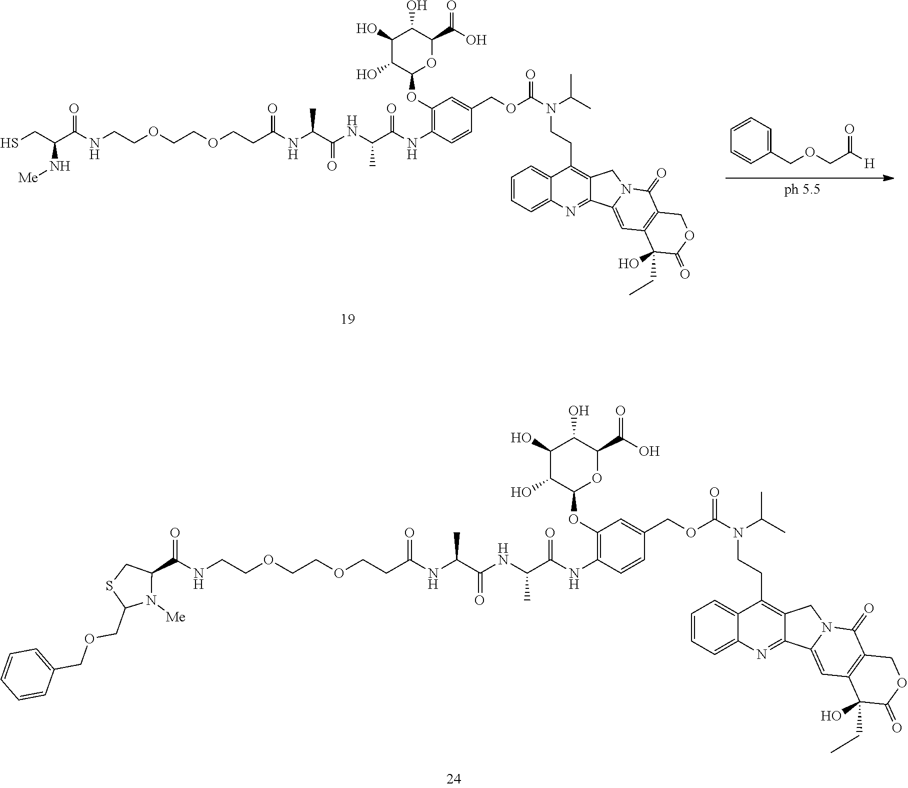 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@H](C)NC(=O)CCOCCOCCNC(=O)[C@H](CS)NC)c(O[C@@H]2O[C@H](C(=O)O)[C@@H](O)[C@H](O)[C@H]2O)c1)C(C)C ; O=CCOCc1ccccc1 ; CC[C@@]1(O)C(=O)OCc2c1cc1n(c2=O)Cc2c-1nc1ccccc1c2CCN(C(=O)OCc1ccc(NC(=O)[C@H](C)NC(=O)[C@H](C)NC(=O)CCOCCOCCNC(=O)[C@@H]2CSC(COCc3ccccc3)N2C)c(O[C@@H]2O[C@H](C(=O)O)[C@@H](O)[C@H](O)[C@H]2O)c1)C(C)C]

### Procedure:

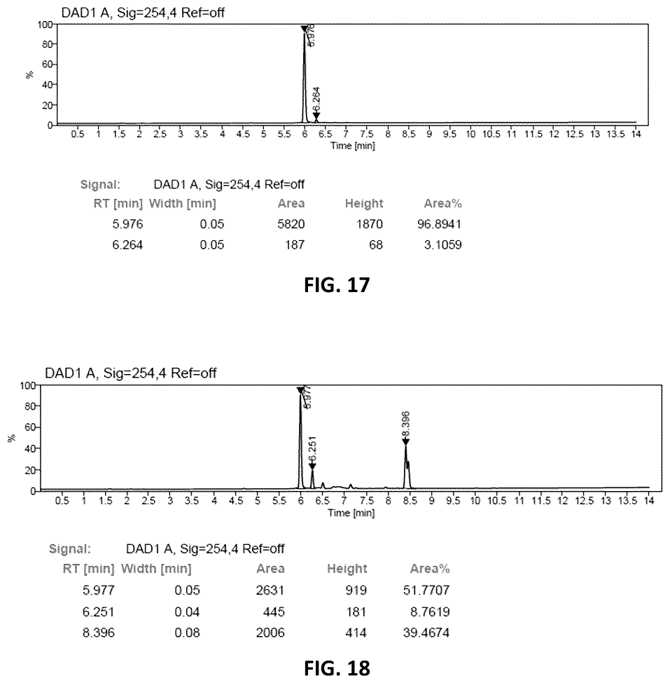

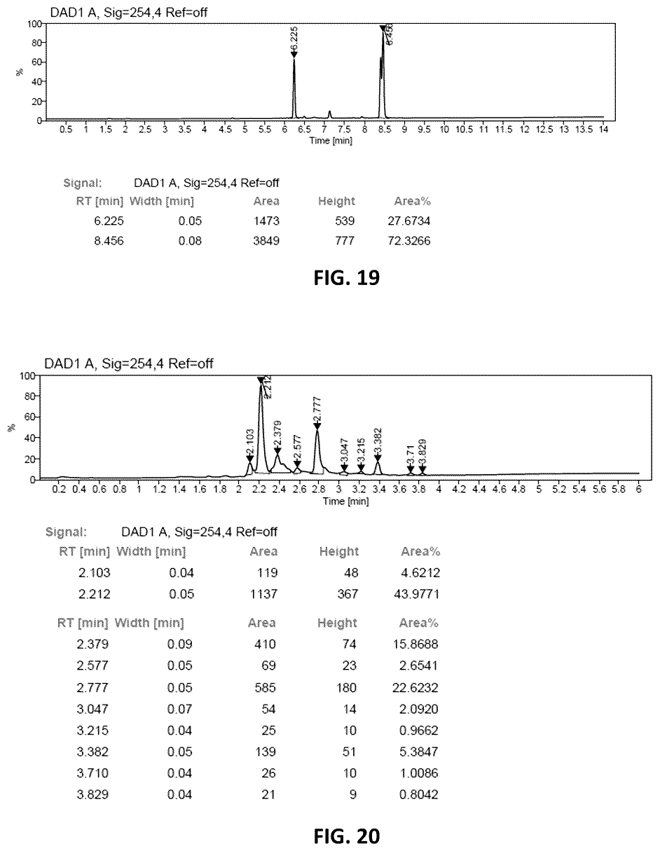

Stock solutions of compound 19 (50 mM in DMA, 1 μL) was diluted with 50 μL of pH 5.5 buffer (20 mM sodium citrate, 50 mM NaCl). To this diluted solution were added of benzyloxyacetaldehyde (BOAA) solution (1 μL, 100 mM in DMA, 2 equiv) at ambient temperature. Reaction progress was monitored by HPLC. Aliquots were drawn and analyzed at TO (FIG. 17), T 30 min (FIG. 18) and T 16h (FIG. 19).

### Results:

Reaction of N-methyl-L-cysteine derivative 19 with benzyloxyacetaldehyde was found significantly slower than previously observed for the N-H L-cysteine congener 12. After 30 mins, only 39% was conjugated to BOAA (as compared to the complete conjugation of 12, see FIG. 13) with remaining 52% N-Me cysteine and 9% oxidation to disulfide. After 16 h, compound 19 was fully consumed giving 72% of conjugation product and 28% of disulfide dimer.

### Example 5

### Stability of BOAA Conjugates 20 and 21 in the Presence of Excess Formaldehyde

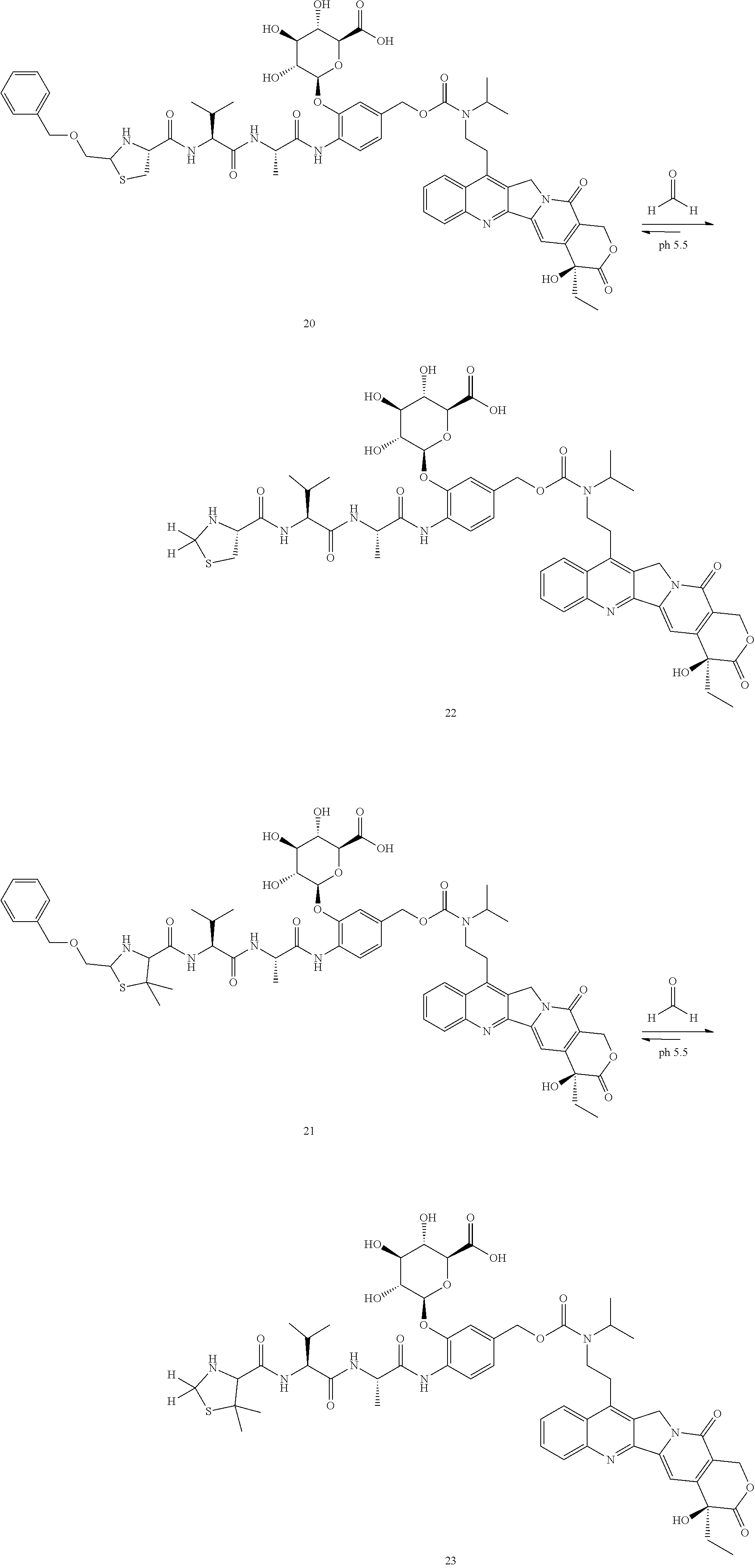

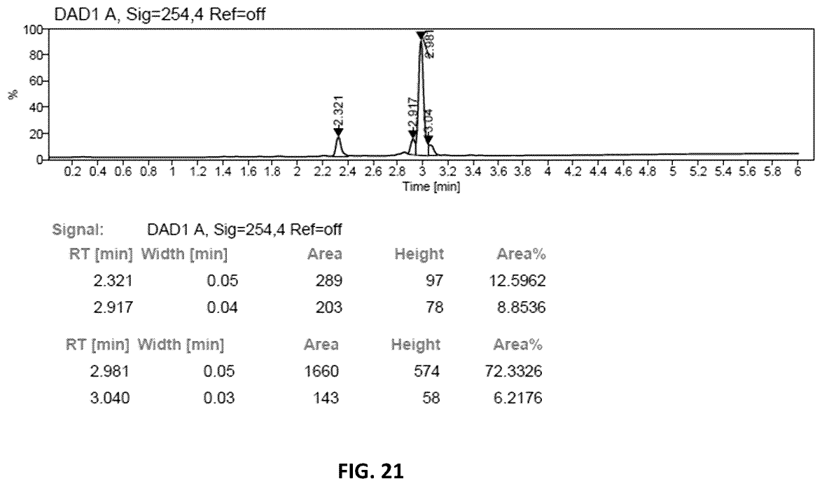

Stock solutions of 12 and 13 (50 mM in DMA, 1 μL) were diluted with 50 μL of pH 5.5 buffer (20 mM sodium citrate, 50 mM NaCl). To this diluted solution were added 1 μL of benzyloxyacetaldehyde (BOAA) solution of DMA (100 mM) at ambient temperature. After 30 min, formation of BOAA conjugates 20 and 21 were complete based on HPLC analysis (FIG. 13 and FIG. 14). Reaction mixtures were treated with 2 μL formaldehyde in water (37%, 13.3 M. ^{˜}150 equiv), incubated at ambient temperature for 24 h and analyzed again by HPLC (FIG. 20 for 20 and FIG. 21 for 21). The identity of formaldehyde adducts 22 and 23 was confirmed by mass LRMS-ESI, while the relative percentage of components in the mixture was determined based on UV traces integration.

### Results:

BOAA conjugate 20 with excess formaldehyde HPLC trace is shown in FIG. 20: Formaldehyde conjugate 22:44% BOAA conjugate 20 remaining: 23%.
BOAA conjugate 21 with excess formaldehyde HPLC trace is shown in FIG. 21: Formaldehyde conjugate 23:13% BOAA conjugate 21 remaining: 81%.

The observed results suggest that under these experimental conditions with excess amount of formaldehyde present thiazolidine compound 21, derived from D-penicillamine, undergoes hydrolysis and re-cyclization at much slower rate, compared to compound 20, derived from L-cysteine, signaling the overall higher stability of 21 towards hydrolysis.

### Example 6

Conjugation of Compounds 5 and 8 with Aldehyde-Tagged Antibodies (HER2, CD33, FITC) General Procedure:

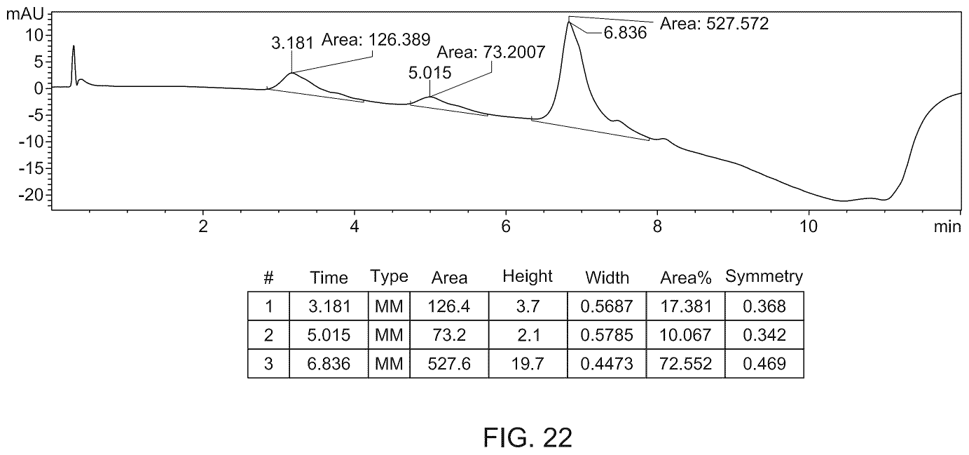

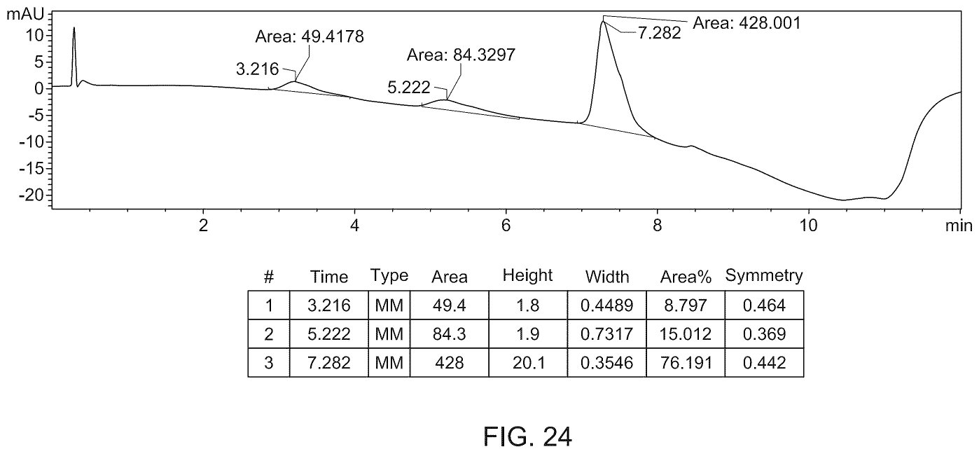

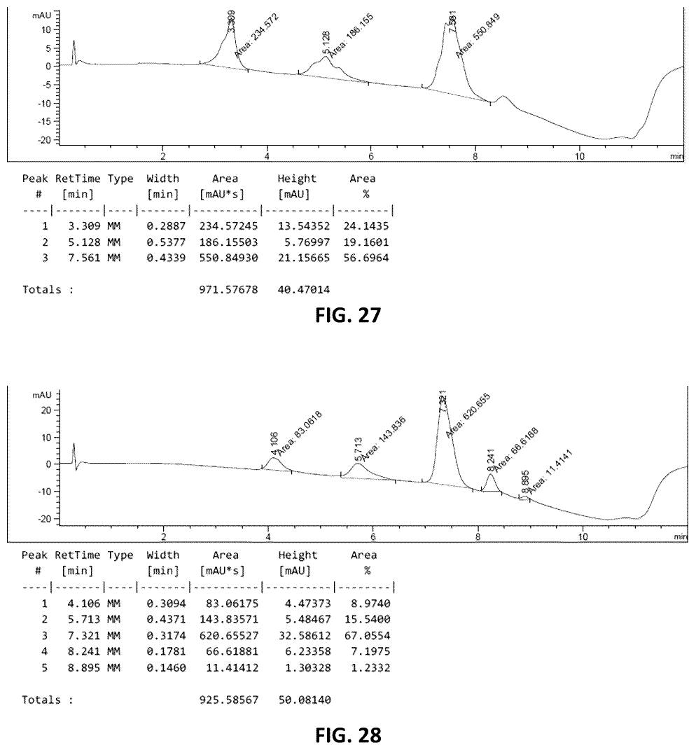

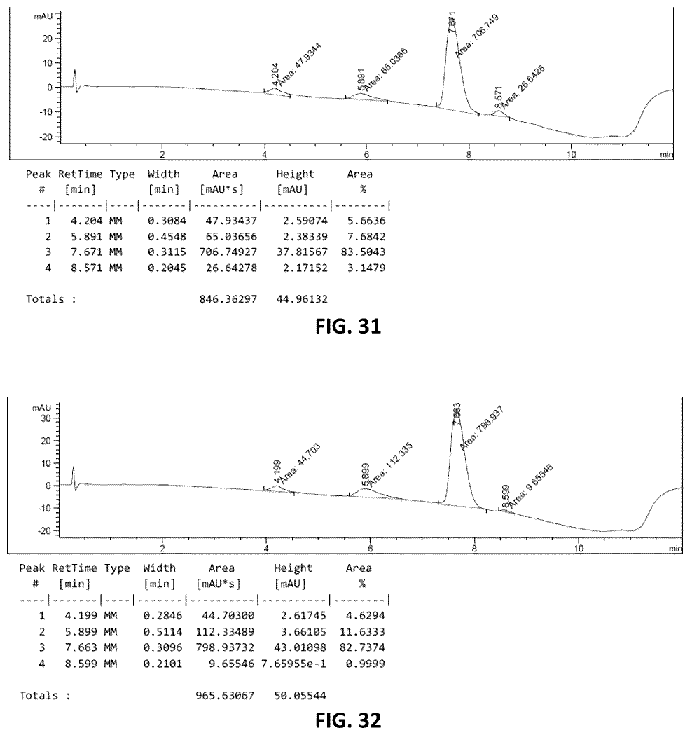

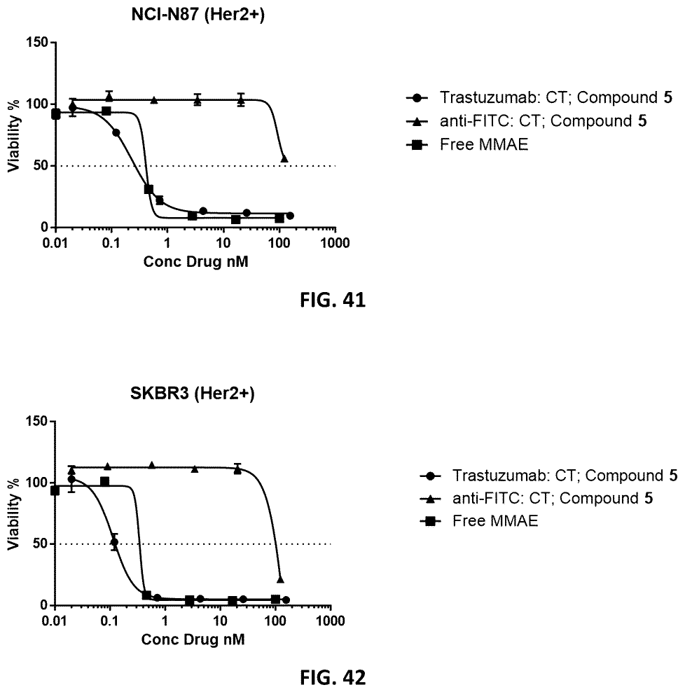

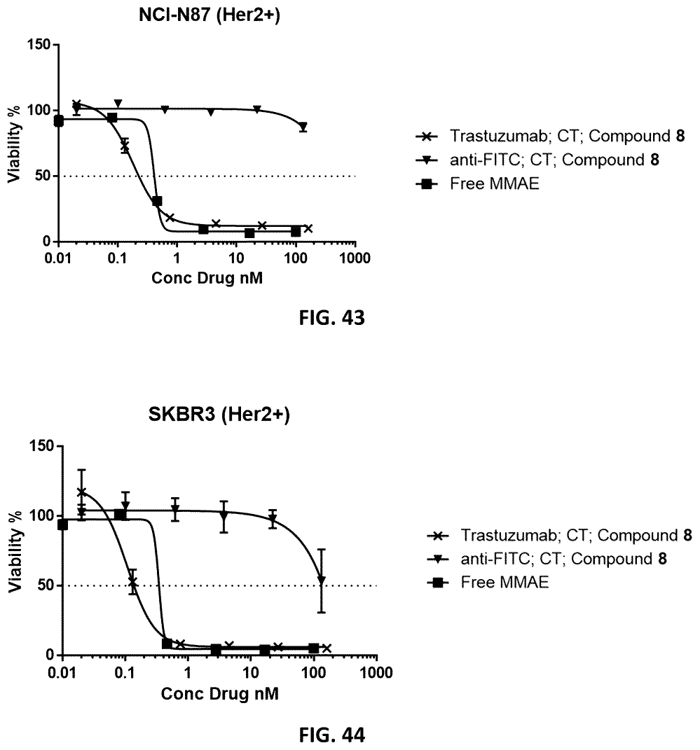

A solution of antibody bearing one aldehyde tag (50 μL) was diluted with pH 5.5 buffer (50 μL, 20 mM sodium citrate, 50 mM NaCl) to final concentration of mAb ranging 9-13 mg/mL. To this mAb solution were added compound 5 or 8 (50-60 mM in DMA, 20 equiv with respect to mAb). The resulting mixture was incubated for 3 days at 37° C. In the case of L-cysteine derivative 12, another 20 equiv of drug-linker were added to the conjugation mixture at 18 h mark, after which the incubation continued (additional fresh drug-linker was needed in this case to account for the loss of 12 due to oxidation, based on the stability experiment above). After conjugation, free drug was removed using a 30 kD MWCO 0.5 mL Amicon spin concentrator. Samples were added to the spin concentrator, centrifuged at 15,000×g for 7 min, then diluted with 450 mL 20 mM sodium citrate, 50 mM NaCl pH 5.5 (20/50 buffer), and centrifuged again. The process was repeated 10 times. The final concentration of ADC was determined by Nanodrop. DAR was determined by HIC (Tosoh #14947) using mobile phase A: 1.5 M ammonium sulfate, 25 mM sodium phosphate pH 7.0, and mobile phase B: 25% isopropanol, 18.75 mM sodium phosphate pH 7.0. HIC traces of ADC HER2/CT-5 and ADC HER2/CT-8 with calculated DAR are shown in FIG. 22 and FIG. 24, respectively. HIC traces of ADC FITC/CT-5 and ADC FITC/CT-8 with calculated DAR are shown in FIG. 26 and FIG. 27, respectively. HIC traces of ADC CD33/CT-5 and ADC CD33/CT-8 with calculated DAR are shown in FIG. 28 and FIG. 31, respectively. The prepared and purified HER2/CT-5 and HER2/CT-8 ADCs were tested for in vitro potency using FITC/CT-5 and FITC/CT-8 conjugates as the isotype controls (FIGS. 41-44).

### Example 7

### Stability of Conjugates CD33/CT-5 and CD33/CT-8 in pH 5.5 20/50 Citrate Buffer

Purified ADCs in pH 5.5 citrate buffer (20 mM sodium citrate, 50 mM NaCl) at 26.1 mg/mL concentration were incubated at 37° C. for up to 72 h. Aliquots were drawn from each sample at TO, T 24 h, and T 72 h and analyzed by HIC to determine DAR at the selected time points. HIC traces of conjugates of 5-CD33 at TO, 24 h and 72 h are shown in FIGS. 28, 29, and 30. HIC traces of conjugates with 8-CD33 at TO, 24 h and 72 h are shown in FIGS. 31, 32, 33.

### Results:

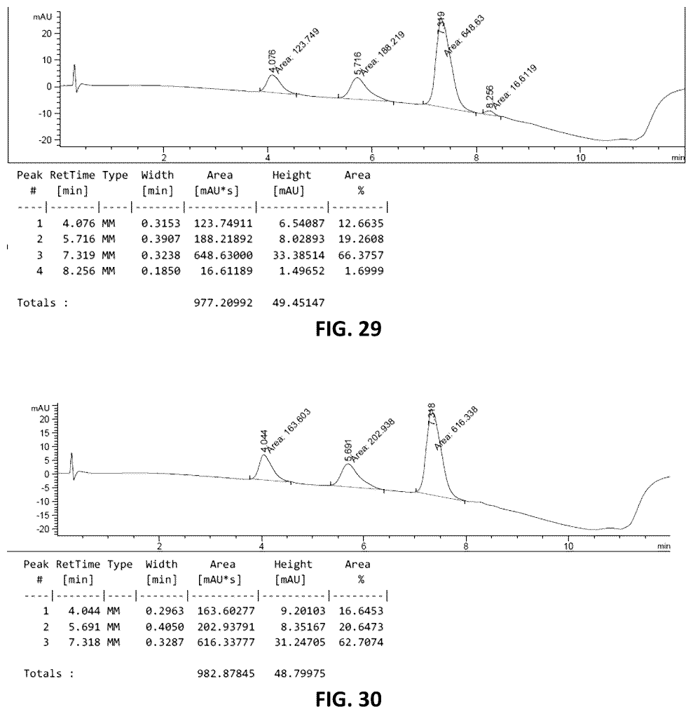

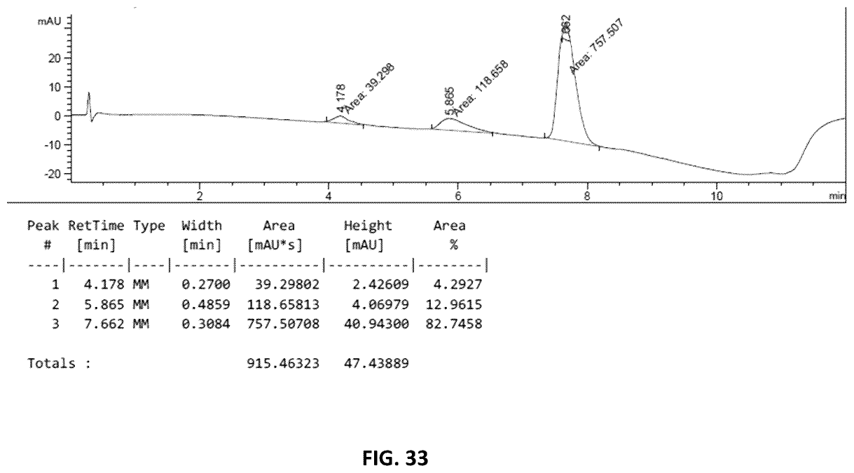

For the conjugate of L-cysteine linker-payload 5, the observed DAR gradually decreased from 1.63 (TO, FIG. 28) to 1.55 (24h, FIG. 29) to 1.46 (72h, FIG. 30). For conjugates of D-penicillamine linker-payload 8, the observed DAR did not significantly change over the course of 3 days (1.80 at TO, FIG. 31 cf. 1.78 at 72h, FIG. 33).

### Example 8

### Stability of Conjugates HER2/CT-5 and HER2/CT-8 in pH 7.4 10× DPBS Buffer

The purified 5-HER2/CT and 8-HER2/CT conjugates in pH 5.5 citrate buffer at 1.4-2 mg/mL (10 μL) were diluted with 10× DPBS pH 7.4 buffer (10 μL) and the resulting buffered solutions were incubated at 37° C. for 6 days, followed by HIC analysis.

### Results:

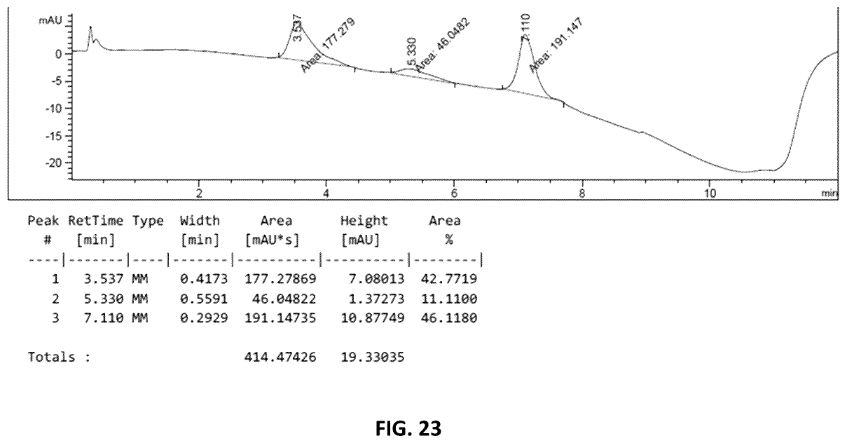

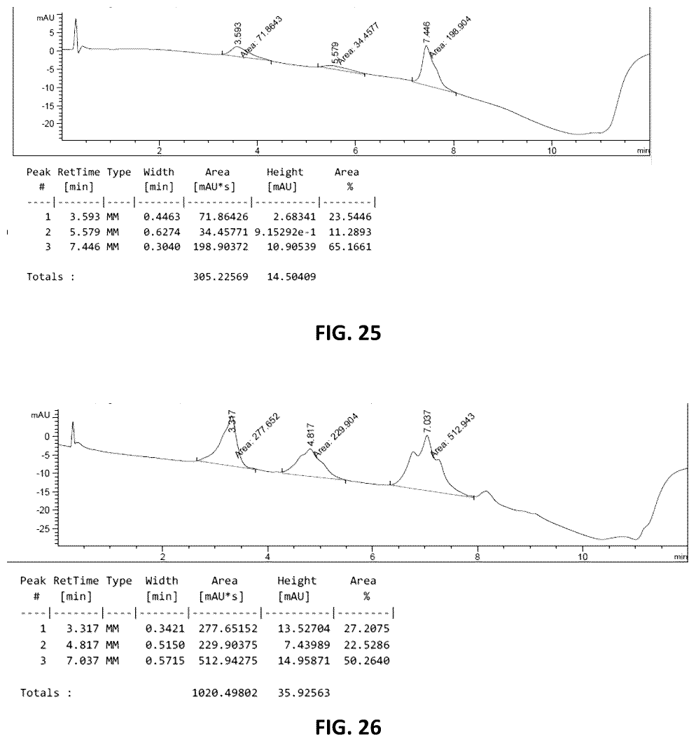

After 6 days, the conjugate of L-cysteine linker-payload 5 showed a decreased DAR (1.56 at TO cf. 1.03 at T 6 d (FIGS. 22 and 23), while the conjugate of D-penicillamine linker-payload 8 was affected to a lesser extent (DAR 1.62 at TO cf. DAR 1.42 at T 6d (FIGS. 24 and 25). This experiment demonstrated higher stability of gem-dimethyl-derived thiazolidine conjugate at physiologically relevant pH levels.

### Example 9

### Optimization of Thiazolidine Bioconjugation for D-Penicillamine Linker-Payload 8 Initial Protocol Ifor Preparing Thiazolidine ADCs

An antibody (final concentration=15 mg/mL) bearing one, two or three aldehyde tags was conjugated to D-penicillamine linker-payload 8 at 0.85 mM or 1.7 mM or 2.0 mM depending on the number of aldehyde tags. The reaction proceeded for 72 h at 37° C. in pH=5.5 citrate buffer (20 mM citrate, 50 mM NaCl). Linker-payload 8 (50 mM stock solution in DMA) was added to the antibody solution, and the resulting mixture was incubated for 72 h at 37° C. After conjugation, the free drug was removed using a 30 kD MWCO 0.5 mL Amicon spin concentrator. Samples were added to the spin concentrator, centrifuged at 15,000×g for 7 min, then diluted with 450 mL 20 mM sodium citrate, 50 mM NaCl pH 5.5 (20/50 buffer), and centrifuged again. The process was repeated 10 times. Drug-to-antibody ratio (DAR) of the obtained ADC was determined by analytical chromatography using HIC (Tosoh #14947) or PLRP-RP (Agilent PL1912-1802 1000A, 8 mm, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, the sample was denatured by adding 50 mM DTT, 4 M guanidine HCl (final concentrations) and heating at 37° C. for 30 min. To determine % aggregation, samples were analyzed using analytical size exclusion chromatography (SEC; Tosoh #08541) with mobile phase comprising 300 mM NaCl, 25 mM sodium phosphate pH 6.8 with 5% isopropanol.

### Results and Observations:

Protocol I provided acceptable DAR and ADC recovery for single aldehyde-tag antibodies (DAR of 1.2-1.5); DARs and yields generally decreased with higher number of aldehyde tags on the antibody.

### Example 10

### Optimized Thiazolidine Conjugation Protocol 11 Based on the Observed Precipitation Formation of the Compound 8 in Conjugation Conditions

An antibody (final concentration=3 mg/mL) bearing one, two or three aldehyde tags was conjugated to D-penicillamine drug-linker 8. The reaction proceeded for 72 h at 37° C. in Acetate/DTPA buffer with 0.4 mM drug-linker at pH=5.5. Acetate/DTPA buffer contains 90 mM NaOAc, 45 mM NaCl and 2 mM DTPA with an adjusted pH of 5.5. The antibody stock solution was 20 mg/mL in Acetate/DTPA buffer at pH 5.5. Stock solution of 8 (50 mM in DMA) was added to the buffer solution of mAb, and the resulting mixture was incubated for 72 h at 37° C. After conjugation, the free drug was removed using a 30 kD MWCO 0.5 mL Amicon spin concentrator. Samples were added to the spin concentrator, centrifuged at 15,000×g for 7 min, then diluted with 450 mL 20 mM sodium citrate, 50 mM NaCl pH 5.5 (20/50 buffer), and centrifuged again. The process was repeated 10 times, followed by HPLC analysis of the obtained ADC solution to determine DAR using HIC (Tosoh #14947) or PLRP-RP (Agilent PL1912-1802 1000A, 8 mm, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, the sample was denatured by adding 50 mM DTT, 4 M guanidine HCl (final concentrations) and heating at 37° C. for 30 min. To determine % aggregation, samples were analyzed using analytical size exclusion chromatography (SEC; Tosoh #08541) with mobile phase of 300 mM NaCl, 25 mM sodium phosphate pH 6.8 with 5% isopropanol.

### Observations and Results for Optimization:

The optimized protocol II results in improved DARs for the thiazolidine reaction.

### Examples are Shown Below

### For Single Aldehyde Tag mAb

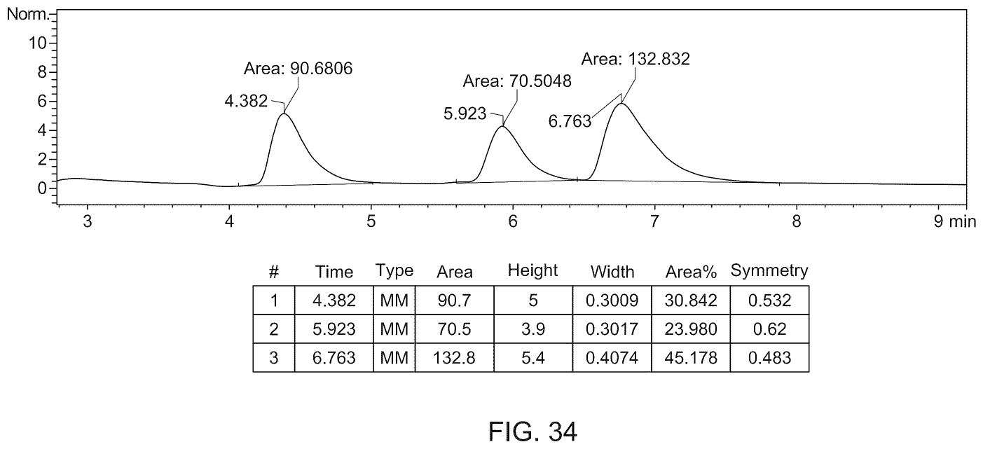

Using initial protocol I: FITC CH1 compound 8 in 20/50 buffer at 3 mg/mL of antibody:
PLRP trace is shown in FIG. 34.
Calculated DAR=1.30

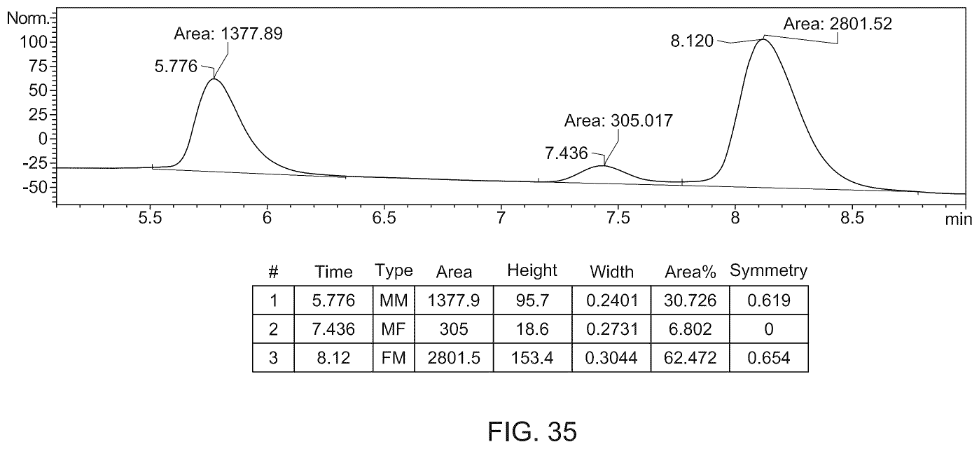

Optimized protocol II: FITC CH1 compound 8 in NaOAc/NaCl/DTPA buffer at 3 mg/mL of antibody:
PLRP trace is shown in FIG. 35.
Calculated DAR=1.80

### For Double-Tagged mAb:

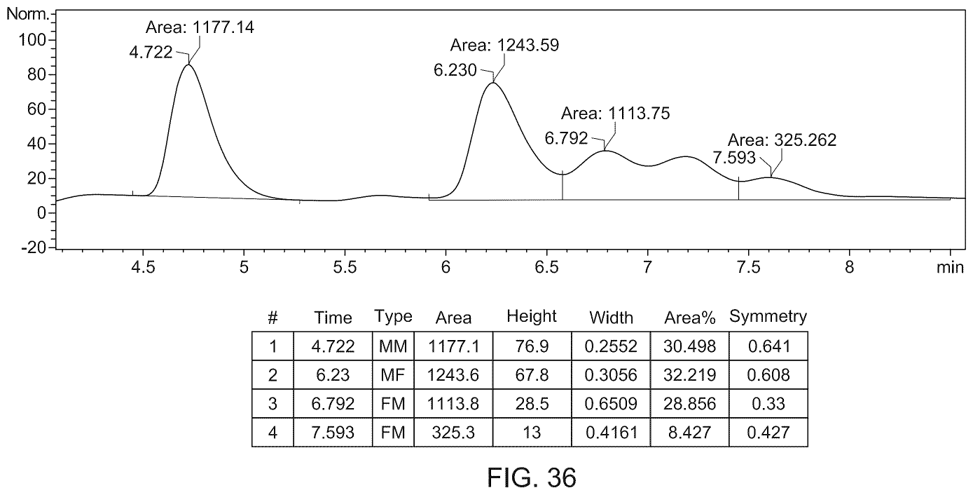

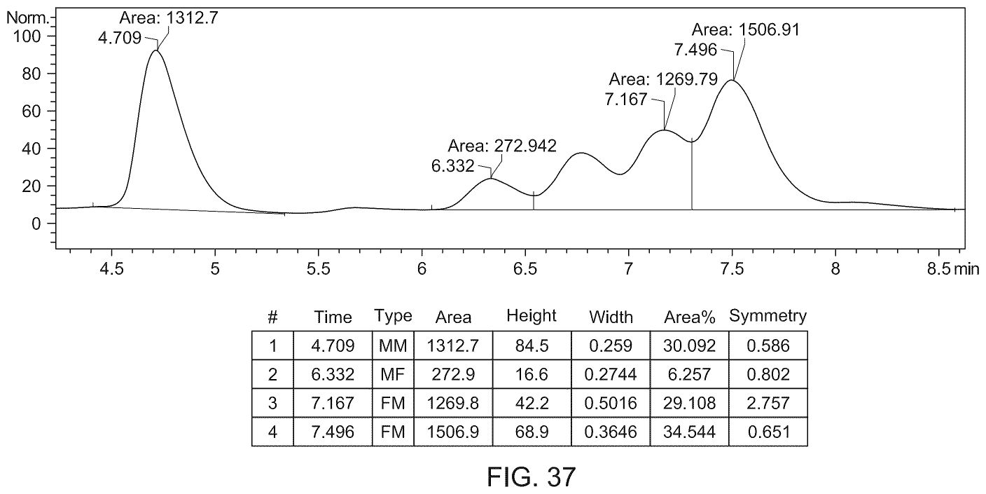

Initial protocol I: HER2 CH1/CT compound 8—in 20/50 buffer, DAR=1.32 PLRP trace is shown in FIG. 36.
Optimized protocol II: HER2 CH1/CT compound 8—in NaOAc/NaCl/DTPA buffer, DAR=2.81
PLRP trace is shown in FIG. 37.

### Example 11

### Conjugation Using Acetonide-Protected D-Penicillamine Compound 14

It has been noticed that acetonide-protected D-penicillamine linker payloads undergo slow hydrolysis spontaneously in aqueous media, especially at low pH. It was hypothesized that slow hydrolysis may provide the basis for measured and steady introduction of reactive aminothiol during conjugation process, effectively minimizing possible oxidation and improving the overall conjugation efficiency.

### Example 12

### Conjugation Procedure for Acetonide-Protected (14) and Free D-Penicillamine (13) Drug-Linkers With Aldehyde-Tagged Antibody

An antibody (final concentration=3 mg/mL) bearing one aldehyde tag was conjugated to D-penicillamine-derived linker-payloads 13 and 14 in free thiol and acetonide forms, respectively. The reaction proceeded for 72 h at 37° C. in Acetate/DTPA buffer with 0.4 mM drug-linker at pH=5.5. Acetate/DTPA buffer contains 90 mM NaOAc, 45 mM NaCl and 2 mM DTPA with an adjusted pH of 5.5. The antibody stock solution was 20 mg/mL in Acetate/DTPA buffer at pH 5.5. The drug-linker stock solution was 50 mM in DMA, which was added to the buffered solution of antibody, followed by incubation for 72 h at 37° C. After conjugation, the free drug was removed using a 30 kD MWCO 0.5 mL Amicon spin concentrator. Samples were added to the spin concentrator, centrifuged at 15,000×g for 7 min, then diluted with 450 mL 20 mM sodium citrate, 50 mM NaCl pH 5.5 (20/50 buffer), and centrifuged again. The process was repeated 10 times. Drug-to-antibody ratio (DAR) of the resulting ADCs was determined by analytical chromatography using PLRP-RP (Agilent PL1912-1802 1000A, 8 mm, 50×2.1 mm) column. PLRP analysis used mobile phase A: 0.1% trifluoroacetic acid in water, and mobile phase B: 0.1% trifluoroacetic acid in acetonitrile. Prior to PLRP analysis, the sample was denatured by adding 50 mM DTT, 4 M guanidine HCl (final concentrations) and heating at 37° C. for 30 min.

### Results:

### 13-Fitc Conjugate:

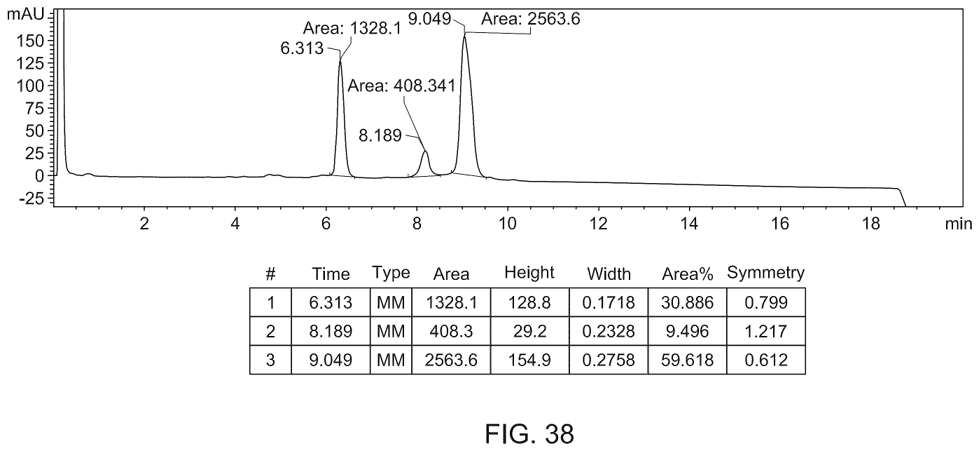

PLRP trace is shown in FIG. 38.

### Calculated DAR=1.73

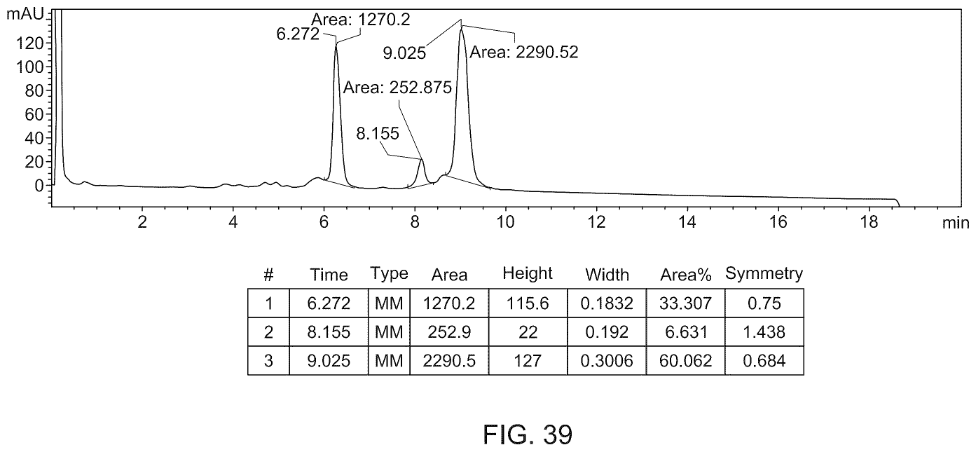

14-FITC conjugate:
PLRP trace is shown in FIG. 39.
Calculated DAR−1.80

Based on PLRP analysis, acetonide-protected D-penicillamine 14 provided equally efficient conjugation in comparison to free penicillamine 13. There was a minor improvement in conjugation on single aldehyde-tagged antibody with acetonide-protected 14 under the chosen reaction conditions.

### Example 13

### In Vitro Cytotoxicity Assays.

Cell lines were plated in 96-well plates (Costar 3610) at a density of 5×104 cells/well in 100 μL of growth media. The next day cells were treated with 20 μL of test articles serially diluted in media. After incubation at 37° C. with 5% CO2 for 5 days, viability was measured using the Promega CellTiter Glo® reagent according to the manufacturer's recommendations. G150 curves were calculated in GraphPad Prism normalized to the payload concentration. The results of in vitro potency for HER2/CT-5 and HER2/CT-8 ADCs along with FITC/CT-5 and FITC/CT-8 controls on HER2-positive NCI-N87 and SKBR3 cell-lines are shown in FIGS. 41-44.

### Example 14

### Serum Stability Assay.

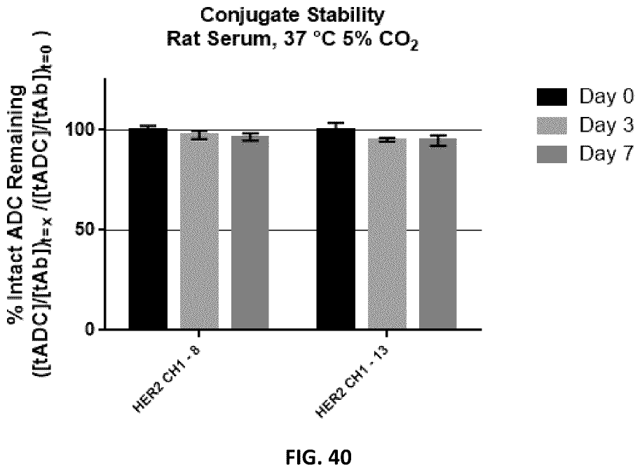

Blank serum matrix was spiked with 40 μg/mL of 8-HER2/CH1 and 13-HER2/CH1 conjugates on day 0. Day 0 stability sample aliquots were placed in −80° C. storage immediately following preparation. The remaining stability sample aliquots were placed in an incubator at 37° C. with 5% CO2, and subsequently placed at −80° C. storage after the allotted time, to produce day 3 and day 7 stability sample aliquots. Samples were thawed once on the day of analysis and analyzed together via total antibody (tAb) and total ADC (tADC) anti-human capture ELISA. The results are presented in FIG. 40 as % of intact ADC concentration and suggest high stability of both conjugates in rat serum with <10% decrease in total concentration after 7 days.

### Example 15

### Sample Analysis (ELISA)

Standard curve samples were prepared by spiking test article reference standard stock into blank serum matrix. Standard curve samples and stability sample aliquots were further diluted 1:33.3 with casein blocking buffer (Minimal Required Dilution (MRD)) to create binding mixture, to yield 3% serum mixture. Binding mixture of standards and samples was added to plates pre-coated with capture reagent (anti-human IgG) and blocked with PBS casein solution. ELISA plates with binding mixtures were then incubated at RT for 60 minutes with shaking at 700 rpm. Plates were washed three times with 300 μL of 0.1% Tween in PBS, followed by the addition of detection antibodies.

For the total antibody assay(tAb), the captured molecules were detected by HRP-conjugated anti-human Fc antibody (100 μL of 0.01 μg/mL), in a 30 min incubation at RT with shaking at 700 rpm. Plates were then washed three times with 300 μL of 0.1% Tween in PBS. The plate-captured immunocomplexes were detected by adding Ultra TMB substrate (100 μL) followed by 2 N H2SO4 (100 μL) to quench. Absorbance was measured at 450 nm on a BioTek Gen 5 plate reader. Data were analyzed in Excel and GraphPad Prism. Concentrations of QC samples were interpolated from the standard curve using a 4-parameter logistic (4 PL) model.

For the total ADC assay(tADC), the captured molecules were detected in 1 hour incubation with a primary mouse anti-drug antibody (100 μL of 0.5 μg/mL), then washed three times with 300 μL of 0.1% Tween in PBS, followed by 30 min incubation with HRP-conjugated anti-mouse IgG1 secondary antibody (100 μL of 0.01 μg/mL). Plates were then washed three times with 300 μL of 0.1% Tween in PBS and the plate-captured immunocomplexes were detected by adding Ultra TMB substrate (100 μL) followed by 2 N H2SO4 (100 μL) to quench. Absorbance was measured at 450 nm on a BioTek Gen 5 plate reader. Data were analyzed in Excel and GraphPad Prism. Data were analyzed in Excel and GraphPad Prism. Concentrations of QC samples were interpolated from the standard curve using a 4-parameter logistic (4 PL) model.

### Example 16

### Pharmacokinetic Studies in Rats

Study Design: Sprague-Dawley rats (3/group) were given a single i.v. bolus dose of 3 mg/kg of ADC comprising trastuzumab conjugated to Compound 8 with DAR of 2. Plasma samples were collected at the designated times and were analyzed for total antibody, total conjugate, and total ADC concentrations.

Standard curve and Quality Control (QC) samples were prepared in neat, pooled serum or plasma from reference stocks of each given ADC. Study samples were pre-diluted to various levels using neat, pooled serum or plasma to yield concentrations within the quantifiable range of the standard curve. Next, Standards (STDs), QCs and pre-diluted study samples were further diluted 1:33.3 with PBS casein buffer (Minimal Required Dilution step), to yield 3% serum in each well. The STDs, QCs and samples were added to plates pre-coated with capture reagent (goat anti-human poly clonal IgG) and blocked with the casein blocking buffer. The plates with binding mixtures were then incubated at RT for 90 minutes with shaking. Plates were washed, followed by the addition of primary and secondary detection antibodies depending on the assay.

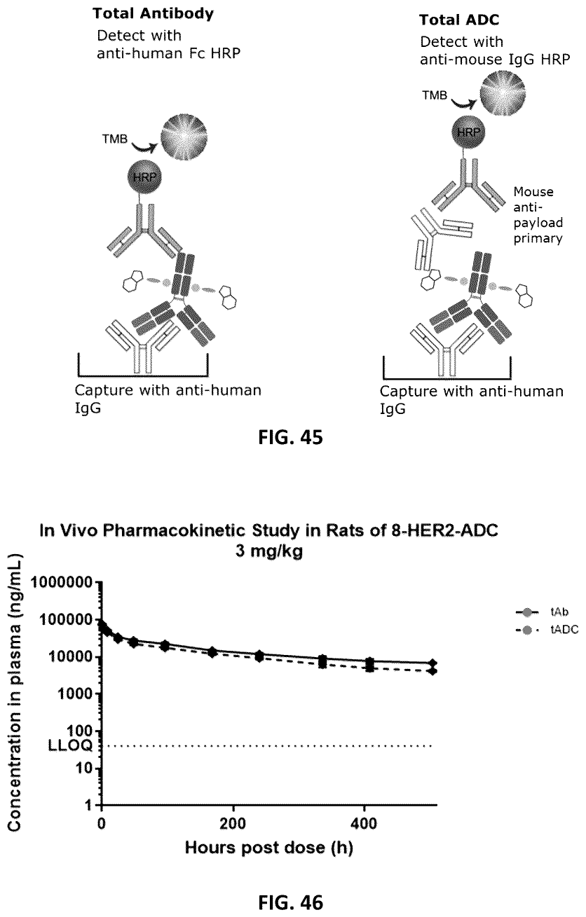

For the total antibody assay (FIG. 45, left panel), the captured molecules were detected by HRP-conjugated anti-human antibody, in a 30-minute incubation period at RT with shaking. For the total ADC assays (FIG. 45, right panel), the captured molecules were detected by a primary mouse anti-Belotecan antibody or mouse anti-MMAE antibody (depending on ADC payload), in a 60-minute incubation period at RT with shaking. Plates were washed, followed by an HRP-conjugated anti-mouse IgG1 secondary detection antibody, in a 30-minute incubation period at RT with shaking. The plate-captured immunocomplexes were detected by adding Ultra TMB substrate followed by 2 N H2SO4 to quench enzymatic activity. Absorbance was measured at 450 nm on a BioTek Gen 5 plate reader. Data were analyzed in Excel and GraphPad Prism.

Concentrations of QC and study samples were interpolated from the standard curve using a 4-parameter logistic (4 PL) model. Lower limit of quantitation (LLOQ) for total antibody and total ADC methods was 40 ng/mL in 100% plasma or serum matrix. Upper limit of quantitation (ULOQ) for total antibody and total ADC methods was 2560 ng/mL in 100% plasma or serum matrix.

Results for the ADC of Compound 8 are shown in FIG. 46, which shows a graph of rat pharmacokinetic data showing total antibody and total ADC concentrations as measured by ELISA in plasma samples from rats dosed with the indicated compounds and sampled at the times shown. Based on the data obtained, the rate of thiazolidine conjugate clearance was comparable to that of the naked antibody, which indicated high stability of the ADC in vivo.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A conjugate of formula (I): 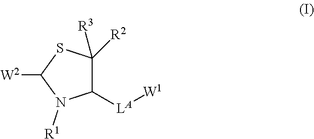
wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker;
W¹ is a drug; and
W² is a peptide.

2. The conjugate of claim 1, wherein R¹ is hydrogen.

3. The conjugate of claim 1, wherein R¹ is alkyl.

4. The conjugate of claim 1, wherein R² and R³ are each hydrogen.

5. The conjugate of claim 1, wherein one of R² and R³ is hydrogen and one of R² and R³ is alkyl.

6. The conjugate of claim 1, wherein R² and R³ are each alkyl.

7. The conjugate of claim 1, wherein L^{A} comprises: 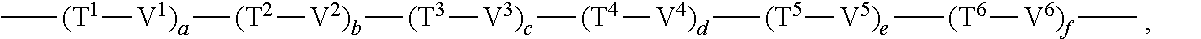
wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³0H)ₘ-, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

8. The conjugate of claim 7, wherein:
(PEG)ₙ is 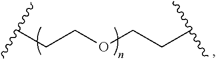
where n is an integer from 1 to 30;
EDA is an ethylene diamine moiety having the following structure: 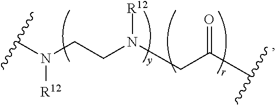
where y is an integer from 1 to 6 and r is 0 or 1;
4-amino-piperidine (4AP) is 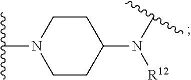
and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

9. The conjugate of claim 7, wherein one of T¹, T², T³, T⁴, T⁵, T⁶, V¹, V², V³, V⁴, V⁵ or V⁶ is a branched group.

10. The conjugate of claim 9, wherein the branched group is selected from —CONR¹⁵— and 4AP.

11. The conjugate of claim 9, wherein the branched group is attached to a compound of formula (II): 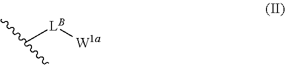
wherein:
L^{B} is a second linker; and
W^{1a} is a drug.

12. The conjugate of claim 11, wherein L^{B} comprises: 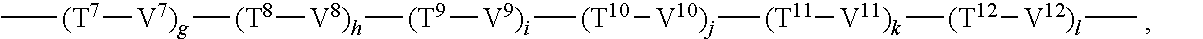
wherein
g, h, i, j, k and I are each independently 0 or 1;
T⁷, T⁸, T⁹, T¹⁰, T¹¹ and T¹² are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³0H)ₘ-, 4-amino-piperidine (4AP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V⁷, V⁸, V⁹, V¹⁰, V¹¹ and V¹² are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

13. The conjugate of claim 11, wherein W¹ and W¹a are the same drug.

14. The conjugate of claim 11, wherein W¹ and W¹a are different drugs.

15. The conjugate of claim 1, wherein the peptide comprises an antibody.

16. The conjugate of claim 1, wherein the conjugate is selected from:

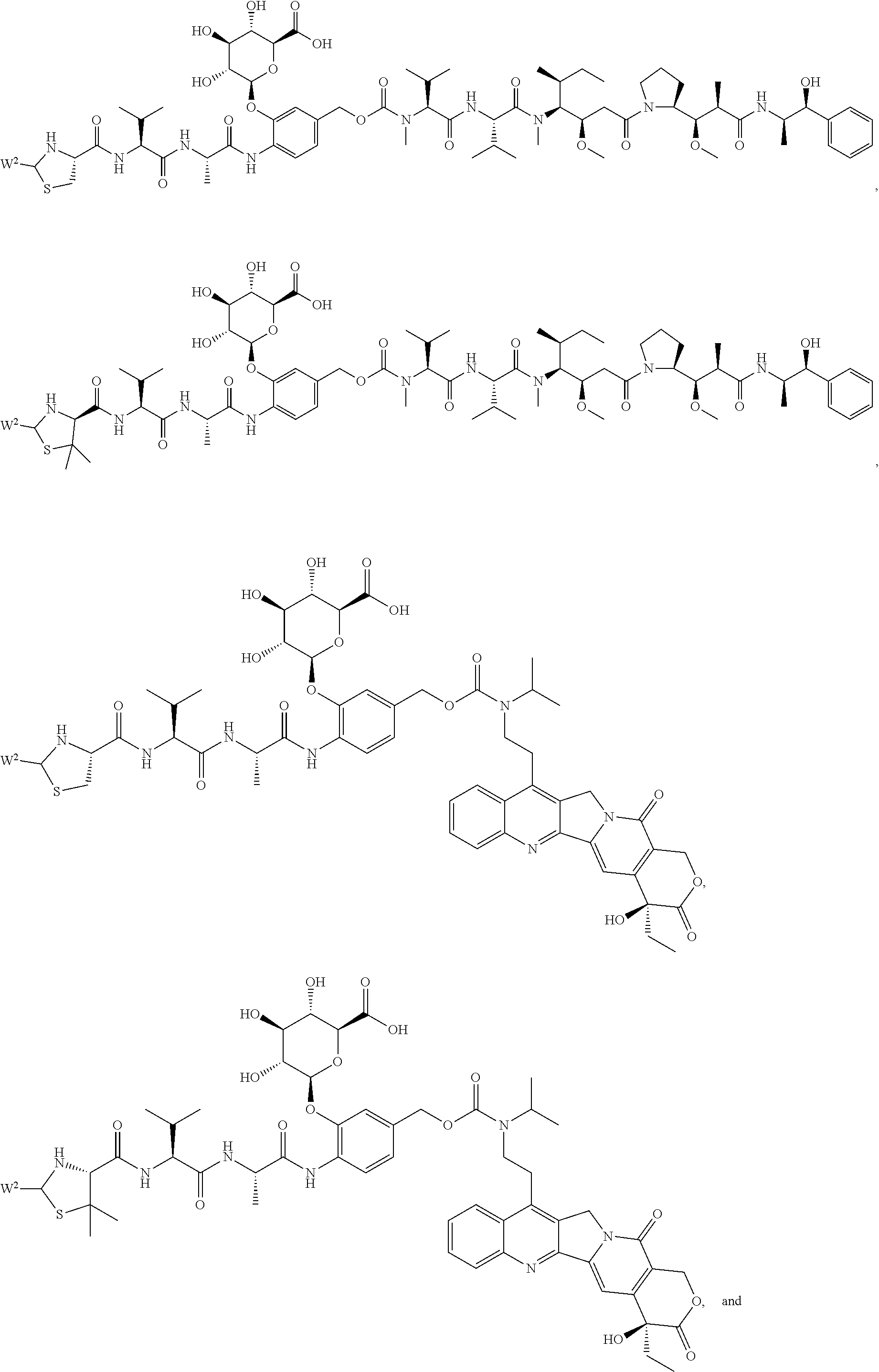

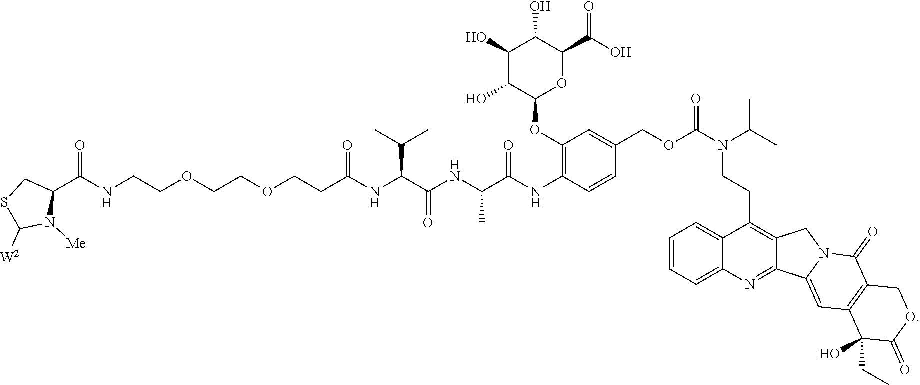

17. A pharmaceutical composition comprising:
a conjugate of claim 1; and
a pharmaceutically acceptable excipient.

18. A method comprising:
administering to a subject a conjugate of claim 1.

19. A method of treating cancer in a subject, the method comprising:
administering to the subject a therapeutically effective amount of a pharmaceutical composition comprising a conjugate of claim 1, wherein the administering is effective to treat cancer in the subject.

20. A method of producing a conjugate according to claim 1, the method comprising:
contacting an aldehyde-tagged peptide with a payload comprising a 1,2-aminothiol group under conditions to produce a conjugate of formula (I): 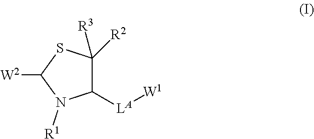
wherein:
R¹ is selected from hydrogen, alkyl, substituted alkyl, alkenyl, substituted alkenyl, alkynyl, and substituted alkynyl;
R² and R³ are each independently selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
L^{A} is a first linker;
W¹ is the payload; and
W² is the peptide.


SEQUENCE LISTING (25 sequences)

SEQ ID NO: 1 - protein, 6 aa, synthetic construct
  VARIANT 2: note = X at position 2 is C or S
LXTPSR

SEQ ID NO: 2 - protein, 6 aa, synthetic construct
  MOD_RES 2: note = X at position 2 is 2-formylglycine or formylglycine
LXTPSR

SEQ ID NO: 3 - protein, 6 aa, synthetic construct
LCTPSR

SEQ ID NO: 4 - protein, 6 aa, synthetic construct
VCTPSR

SEQ ID NO: 5 - protein, 6 aa, synthetic construct
LCSPSR

SEQ ID NO: 6 - protein, 6 aa, synthetic construct
LCAPSR

SEQ ID NO: 7 - protein, 6 aa, synthetic construct
LCVPSR

SEQ ID NO: 8 - protein, 6 aa, synthetic construct
LCGPSR

SEQ ID NO: 9 - protein, 6 aa, synthetic construct
ICTPAR

SEQ ID NO: 10 - protein, 6 aa, synthetic construct
LCTPSK

SEQ ID NO: 11 - protein, 6 aa, synthetic construct
MCTPSK

SEQ ID NO: 12 - protein, 6 aa, synthetic construct
VCTPSK

SEQ ID NO: 13 - protein, 6 aa, synthetic construct
LCSPSK

SEQ ID NO: 14 - protein, 6 aa, synthetic construct
LCAPSK

SEQ ID NO: 15 - protein, 6 aa, synthetic construct
LCVPSK

SEQ ID NO: 16 - protein, 6 aa, synthetic construct
LCGPSK

SEQ ID NO: 17 - protein, 6 aa, synthetic construct
LCTPSA

SEQ ID NO: 18 - protein, 6 aa, synthetic construct
ICTPAA

SEQ ID NO: 19 - protein, 6 aa, synthetic construct
MCTPSA

SEQ ID NO: 20 - protein, 6 aa, synthetic construct
VCTPSA

SEQ ID NO: 21 - protein, 6 aa, synthetic construct
LCSPSA

SEQ ID NO: 22 - protein, 6 aa, synthetic construct
LCAPSA

SEQ ID NO: 23 - protein, 6 aa, synthetic construct
LCVPSA

SEQ ID NO: 24 - protein, 6 aa, synthetic construct
LCGPSA

SEQ ID NO: 25 - protein, 6 aa, synthetic construct
MCTPSR